(19) United States
(12) Patent Application Publication
(10) Pub. No.: US 2026/0144755 A1
(43) Pub. Date: May 28, 2026

(54) Solid Oral Dosage Forms Comprising Tavapadon

(71) Applicant: Cerevel Therapeutics, LLC, North Chicago, IL (US)

(72) Inventors: Hillary Lawton, Framingham, MA (US); Nigel Metcalfe, South Boston, MA (US)

(73) Assignee: Cerevel Therapeutics, LLC, North Chicago, IL (US)

(21) Appl. No.: 19/350,126

(22) Filed: Oct. 6, 2025

Related U.S. Application Data
(60) Provisional application No. 63/703,836, filed on Oct. 4, 2024.

(51) Int. Cl. A61K 9/20; A61K 9/00; A61K 9/28; A61K 31/513

(52) U.S. Cl. CPC A61K 9/2054; A61K 9/0053; A61K 9/2009; A61K 9/2018; A61K 9/2095; A61K 9/28; A61K 31/513

(57) ABSTRACT

The present disclosure provides for pharmaceutical tablets comprising tavapadon and processes for preparing such dosage forms.

### CROSS REFERENCE TO RELATED APPLICATION

This application claims the priority benefit of U.S. Provisional Application No. 63/703,836, filed on Oct. 4, 2024, which is hereby incorporated by reference herein in its entirety.

### FIELD

The present disclosure pertains to pharmaceutical tablets comprising tavapadon and processes for preparing such dosage forms.

### BACKGROUND

Parkinson's disease (PD) is a progressive neurodegenerative disease characterized by deficits in motor control arising from the loss of dopaminergic neurons in the substantia nigra pars compacta and subsequent depletion of dopamine in motor regions of the striatum. Poewe et al., Nat Rev Dis Primers 2017; 3(1): 17013. Standard treatment remains the restoration of dopaminergic function through administration of the synthetic dopamine precursor levodopa (1-dopa) and subsequent activation of D1-like and D2-like dopamine receptors. Rizek et al., CMAJ 2016; 188: 1157-1165; Charvin et al., Nat Rev Drug Discov 2018; 17: 804-822; and Ossig C et al., Neurol Clin 2015; 33: 19-37. However, while use of levodopa provides efficacy and is generally well tolerated, long-term treatment can lead to fluctuating motor responses and dyskinesias in many individuals. Bastide et al., Prog Neurobiol 2015; 132: 96-168.

There has long been interest in the potential of D1- or D2-selective receptor agonists as an alternative to levodopa in subjects with Parkinson's disease. Clinical trials of limited duration with prototype full D1 agonists demonstrated that, while these agents exhibited robust anti-parkinsonian effects on motor symptoms challenges with pharmacokinetics, cardiovascular parameters, and side effects including dyskinesias made them ill-suited for further clinical advancement. Rascol et al., Ann Neurol 1999; 45:736-741. In contrast, efforts to develop D2-selective agonists with suitable clinical properties were more successful, with several receiving approval for the treatment of Parkinson's disease, including pramipexole, ropinirole, and rotigotine. In general, these agents exhibit less-frequent motor complications than levodopa but are not as effective for controlling motor symptoms. Dietrichs et al., Acta Neurol Scand 2017; 136: 378-385. D2-selective agonists are also associated with an increased incidence of specific adverse effects (AEs), including nausea, somnolence, hypotension, compulsive behaviors, and hallucinations, particularly in the elderly. Stowe et al., Cochrane Database Syst Rev 2008: 2: CD006564. Thus, there remains a need for agents that provide effective and predictable control of motor symptoms while avoiding side effects associated with activation of D2-like receptors.

Existing clinical and preclinical data provide theoretical support for the long-standing hypothesis that selective activation of D1-like receptors may be an important therapeutic option for Parkinson's disease. Kim et al., Front Biol (Beijing) 2015; 10: 230-238. To date, however, attempts to develop D1-selective agonists have been hampered by tolerability issues (notably, acute hypotension) and poor pharmacokinetics (e.g. short half-life and low oral availability).

Tavapadon is a D1/D5 dopamine receptor agonist being investigated for the treatment of Parkinson's disease. By targeting the D1 dopamine receptor, tavapadon aims to improve motor control symptoms while minimizing adverse events (AEs) that may be mechanistically linked to D2/D3 dopamine receptor agonists, such as L-Dopa. There is continuing need to develop formulations for tavapadon for more effective treatment of Parkinson's disease to provide sustained relief of motor symptoms with a reduced likelihood of adverse events.

Accordingly, there remains a need in the art for pharmaceutical tablets suitable for administering tavapadon to patients with Parkinson's disease.

### SUMMARY

One aspect of the invention pertains to a pharmaceutical tablet comprising: about 0.25% by weight to about 15% by weight of tavapadon free base; and at least about 80% by weight of at least one filler.

One aspect of the invention pertains to a pharmaceutical tablet comprising: about 0.25% by weight to about 5% by weight of tavapadon free base; and at least about 90% by weight of at least one filler.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is cellulose.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is cellulose.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is dibasic calcium phosphate.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is dibasic calcium phosphate.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is cellulose; wherein the cellulose is microcrystalline cellulose.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is cellulose; wherein the cellulose is microcrystalline cellulose.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is lactose.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is lactose.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is lactose; wherein the lactose is lactose monohydrate.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler is lactose; wherein the lactose is lactose monohydrate.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate; wherein the filler comprises about 59% to about 64% by weight of microcrystalline cellulose and about 31% to about 33% by weight of lactose monohydrate.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate; wherein the filler comprises about 59% to about 64% by weight of microcrystalline cellulose and about 31% to about 33% by weight of lactose monohydrate.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate; wherein the filler comprises about 59% to about 64% by weight of microcrystalline cellulose and about 31% to about 33% by weight of lactose monohydrate; wherein a coating is added to the pharmaceutical tablet.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate; wherein the filler comprises about 59% to about 64% by weight of microcrystalline cellulose and about 31% to about 33% by weight of lactose monohydrate; wherein a coating is added to the pharmaceutical tablet.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate; wherein the filler comprises about 59% to about 64% by weight of microcrystalline cellulose and about 31% to about 33% by weight of lactose monohydrate; wherein a coating is added to the pharmaceutical tablet; wherein the coating comprises a polymer coating.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate; wherein the filler comprises about 59% to about 64% by weight of microcrystalline cellulose and about 31% to about 33% by weight of lactose monohydrate; wherein a coating is added to the pharmaceutical tablet; wherein the coating comprises a polymer coating.

Another aspect of the invention pertains to a pharmaceutical tablet comprising: about 0.25% by weight to about 15% by weight of tavapadon free base; and at least about 80% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate; wherein the filler comprises about 59% to about 64% by weight of microcrystalline cellulose and about 31% to about 33% by weight of lactose monohydrate; wherein a coating is added to the pharmaceutical tablet; wherein the coating comprises a polymer coating; wherein the coating comprises polyvinyl alcohol, polyethylene glycol, titanium dioxide, and talc.

Another aspect of the invention pertains to a pharmaceutical tablet comprising: about 0.25% by weight to about 5% by weight of tavapadon free base; and at least about 90% by weight of at least one filler, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof; wherein at least one filler comprises cellulose and lactose; wherein the cellulose is microcrystalline cellulose and the lactose is lactose monohydrate; wherein the filler comprises about 59% to about 64% by weight of microcrystalline cellulose and about 31% to about 33% by weight of lactose monohydrate; wherein a coating is added to the pharmaceutical tablet; wherein the coating comprises a polymer coating; wherein the coating comprises polyvinyl alcohol, polyethylene glycol, titanium dioxide, and talc.

Another aspect of the invention pertains to a pharmaceutical tablet comprising: about 0.25% by weight to about 15% by weight of tavapadon free base; and at least about 80% by weight of at least one filler; further comprising at least one disintegrant.

Another aspect of the invention pertains to a pharmaceutical tablet comprising: about 0.25% by weight to about 5% by weight of tavapadon free base; and at least about 90% by weight of at least one filler; further comprising at least one disintegrant.

Another aspect of the invention pertains to a pharmaceutical tablet comprising: about 0.25% by weight to about 15% by weight of tavapadon free base; and at least about 80% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof.

Another aspect of the invention pertains to a pharmaceutical tablet comprising: about 0.25% by weight to about 5% by weight of tavapadon free base; and at least about 90% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof.

Another aspect of the invention pertains to a pharmaceutical tablet comprising: about 0.25% by weight to about 15% by weight of tavapadon free base; and at least about 80% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight.

Another aspect of the invention pertains to a pharmaceutical tablet comprising: about 0.25% by weight to about 5% by weight of tavapadon free base; and at least about 90% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight.

Another aspect of the invention pertains to a pharmaceutical tablet comprising: about 0.25% by weight to about 15% by weight of tavapadon free base; and at least about 80% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight; wherein at least one disintegrant is sodium starch glycolate.

Another aspect of the invention pertains to a pharmaceutical tablet comprising: about 0.25% by weight to about 5% by weight of tavapadon free base; and at least about 90% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight; wherein at least one disintegrant is sodium starch glycolate.

Another aspect of the invention pertains to a pharmaceutical tablet comprising: about 0.25% by weight to about 15% by weight of tavapadon free base; and at least about 80% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight; wherein at least one disintegrant is sodium starch glycolate; wherein a coating is added to the pharmaceutical tablet.

Another aspect of the invention pertains to a pharmaceutical tablet comprising: about 0.25% by weight to about 5% by weight of tavapadon free base; and at least about 90% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight; wherein at least one disintegrant is sodium starch glycolate; wherein a coating is added to the pharmaceutical tablet.

Another aspect of the invention pertains to a pharmaceutical tablet comprising: about 0.25% by weight to about 15% by weight of tavapadon free base; and at least about 80% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight; wherein at least one disintegrant is sodium starch glycolate; wherein a coating is added to the pharmaceutical tablet; wherein the coating comprises a polymer coating.

Another aspect of the invention pertains to a pharmaceutical tablet comprising: about 0.25% by weight to about 5% by weight of tavapadon free base; and at least about 90% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight; wherein at least one disintegrant is sodium starch glycolate; wherein a coating is added to the pharmaceutical tablet; wherein the coating comprises a polymer coating.

Another aspect of the invention pertains to a pharmaceutical tablet comprising: about 0.25% by weight to about 15% by weight of tavapadon free base; and at least about 80% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight; wherein at least one disintegrant is sodium starch glycolate; wherein a coating is added to the pharmaceutical tablet; wherein the coating comprises a polymer coating; wherein the coating comprises polyvinyl alcohol, polyethylene glycol, titanium dioxide, and talc.

Another aspect of the invention pertains to a pharmaceutical tablet comprising: about 0.25% by weight to about 5% by weight of tavapadon free base; and at least about 90% by weight of at least one filler; further comprising at least one disintegrant; wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; wherein at least one disintegrant is present in an amount of about 3% by weight; wherein at least one disintegrant is sodium starch glycolate; wherein a coating is added to the pharmaceutical tablet; wherein the coating comprises a polymer coating; wherein the coating comprises polyvinyl alcohol, polyethylene glycol, titanium dioxide, and talc.

Another aspect of the invention pertains to a pharmaceutical tablet comprising: about 0.25% by weight to about 15% by weight of tavapadon free base; and at least about 80% by weight of at least one filler; further comprising at least one lubricant.

Another aspect of the invention pertains to a pharmaceutical tablet comprising: about 0.25% by weight to about 5% by weight of tavapadon free base; and at least about 90% by weight of at least one filler; further comprising at least one lubricant.

Another aspect of the invention pertains to a pharmaceutical tablet comprising: about 0.25% by weight to about 15% by weight of tavapadon free base; and at least about 80% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof.

Another aspect of the invention pertains to a pharmaceutical tablet comprising: about 0.25% by weight to about 5% by weight of tavapadon free base; and at least about 90% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof.

Another aspect of the invention pertains to a pharmaceutical tablet comprising: about 0.25% by weight to about 15% by weight of tavapadon free base; and at least about 80% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight.

Another aspect of the invention pertains to a pharmaceutical tablet comprising: about 0.25% by weight to about 5% by weight of tavapadon free base; and at least about 90% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight.

Another aspect of the invention pertains to a pharmaceutical tablet comprising: about 0.25% by weight to about 15% by weight of tavapadon free base; and at least about 80% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight; wherein at least one lubricant is magnesium stearate.

Another aspect of the invention pertains to a pharmaceutical tablet comprising: about 0.25% by weight to about 5% by weight of tavapadon free base; and at least about 90% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight; wherein at least one lubricant is magnesium stearate.

Another aspect of the invention pertains to a pharmaceutical tablet comprising: about 0.25% by weight to about 15% by weight of tavapadon free base; and at least about 80% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight; wherein at least one lubricant is magnesium stearate; wherein a coating is added to the pharmaceutical tablet.

Another aspect of the invention pertains to a pharmaceutical tablet comprising: about 0.25% by weight to about 5% by weight of tavapadon free base; and at least about 90% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight; wherein at least one lubricant is magnesium stearate; wherein a coating is added to the pharmaceutical tablet.

Another aspect of the invention pertains to a pharmaceutical tablet comprising: about 0.25% by weight to about 15% by weight of tavapadon free base; and at least about 80% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight; wherein at least one lubricant is magnesium stearate; wherein a coating is added to the pharmaceutical tablet; wherein the coating comprises a polymer coating.

Another aspect of the invention pertains to a pharmaceutical tablet comprising: about 0.25% by weight to about 5% by weight of tavapadon free base; and at least about 90% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight; wherein at least one lubricant is magnesium stearate; wherein a coating is added to the pharmaceutical tablet; wherein the coating comprises a polymer coating.

Another aspect of the invention pertains to a pharmaceutical tablet comprising: about 0.25% by weight to about 15% by weight of tavapadon free base; and at least about 80% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight; wherein at least one lubricant is magnesium stearate; wherein a coating is added to the pharmaceutical tablet; wherein the coating comprises a polymer coating; wherein the coating comprises polyvinyl alcohol, polyethylene glycol, titanium dioxide, and talc.

Another aspect of the invention pertains to a pharmaceutical tablet comprising: about 0.25% by weight to about 5% by weight of tavapadon free base; and at least about 90% by weight of at least one filler; further comprising at least one lubricant; wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one lubricant is present in an amount of about 1% by weight; wherein at least one lubricant is magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; wherein a coating is added to the pharmaceutical tablet.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; wherein a coating is added to the pharmaceutical tablet.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; wherein a coating is added to the pharmaceutical tablet; wherein the coating comprises a polymer coating.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; wherein a coating is added to the pharmaceutical tablet; wherein the coating comprises a polymer coating.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon; about 63.83 mg of microcrystalline cellulose; about 31.92 mg of lactose monohydrate; about 3 mg sodium starch glycolate; and about 1 mg magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon; about 63.83 mg of microcrystalline cellulose; about 31.92 mg of lactose monohydrate; about 3 mg sodium starch glycolate; and about 1 mg magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon; about 63.83 mg of microcrystalline cellulose; about 31.92 mg of lactose monohydrate; about 3 mg sodium starch glycolate; and about 1 mg magnesium stearate; wherein a coating is added to the pharmaceutical tablet.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon; about 63.83 mg of microcrystalline cellulose; about 31.92 mg of lactose monohydrate; about 3 mg sodium starch glycolate; and about 1 mg magnesium stearate; wherein a coating is added to the pharmaceutical tablet.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon; about 63.83 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon; about 63.83 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon; about 63.83 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 0.25% by weight of tavapadon; about 64% by weight of microcrystalline cellulose; about 32% by weight of lactose monohydrate; about 3% sodium starch glycolate; and about 1% magnesium stearate; wherein the pharmaceutical tablet comprises about 0.25 mg of tavapadon; about 63.83 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon; about 63.08 mg of microcrystalline cellulose; about 31.92 mg of lactose monohydrate; about 3 mg sodium starch glycolate; and about 1 mg magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon; about 63.08 mg of microcrystalline cellulose; about 31.92 mg of lactose monohydrate; about 3 mg sodium starch glycolate; and about 1 mg magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon; about 63.08 mg of microcrystalline cellulose; about 31.92 mg of lactose monohydrate; about 3 mg sodium starch glycolate; and about 1 mg magnesium stearate; wherein a coating is added to the pharmaceutical tablet.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon; about 63.08 mg of microcrystalline cellulose; about 31.92 mg of lactose monohydrate; about 3 mg sodium starch glycolate; and about 1 mg magnesium stearate; wherein a coating is added to the pharmaceutical tablet.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon; about 63.08 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon; about 63.08 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon; about 63.08 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate; comprising: about 1% by weight of tavapadon; about 63% by weight of microcrystalline cellulose; about 32% by weight of lactose monohydrate; about 3% sodium starch glycolate; and about 1% magnesium stearate; wherein the pharmaceutical tablet comprises about 1 mg of tavapadon; about 63.08 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon; wherein the pharmaceutical tablet comprises about 59.08 mg of microcrystalline cellulose; about 31.92 mg of lactose monohydrate; about 3 mg sodium starch glycolate; and about 1 mg magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon; wherein the pharmaceutical tablet comprises about 59.08 mg of microcrystalline cellulose; about 31.92 mg of lactose monohydrate; about 3 mg sodium starch glycolate; and about 1 mg magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises 5 mg of tavapadon; wherein the pharmaceutical tablet comprises about 59.08 mg of microcrystalline cellulose; about 31.92 mg of lactose monohydrate; about 3 mg sodium starch glycolate; and about 1 mg magnesium stearate; wherein a coating is added to the pharmaceutical tablet.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon; wherein the pharmaceutical tablet comprises about 59.08 mg of microcrystalline cellulose; about 31.92 mg of lactose monohydrate; about 3 mg sodium starch glycolate; and about 1 mg magnesium stearate; wherein a coating is added to the pharmaceutical tablet.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon; wherein the pharmaceutical tablet comprises about 59.08 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon; wherein the pharmaceutical tablet comprises about 59.08 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon; wherein the pharmaceutical tablet comprises about 59.08 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 5 mg of tavapadon; wherein the pharmaceutical tablet comprises about 59.08 mg of microcrystalline cellulose; about 31.92 mg of lactose monohydrate; about 3 mg sodium starch glycolate; and about 1 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon; wherein the pharmaceutical tablet comprises about 177.24 mg of microcrystalline cellulose; about 95.76 mg of lactose monohydrate; about 9 mg sodium starch glycolate; and about 3 mg magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon; wherein the pharmaceutical tablet comprises about 177.24 mg of microcrystalline cellulose; about 95.76 mg of lactose monohydrate; about 9 mg sodium starch glycolate; and about 3 mg magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon; wherein the pharmaceutical tablet comprises about 177.24 mg of microcrystalline cellulose; about 95.76 mg of lactose monohydrate; about 9 mg sodium starch glycolate; and about 3 mg magnesium stearate; wherein a coating is added to the pharmaceutical tablet.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon; wherein the pharmaceutical tablet comprises about 177.24 mg of microcrystalline cellulose; about 95.76 mg of lactose monohydrate; about 9 mg sodium starch glycolate; and about 3 mg magnesium stearate; wherein a coating is added to the pharmaceutical tablet.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon; wherein the pharmaceutical tablet comprises about 177.24 mg of microcrystalline cellulose; about 95.76 mg of lactose monohydrate; about 9 mg sodium starch glycolate; and about 3 mg magnesium stearate; wherein a coating is added to the pharmaceutical tablet; wherein the coating comprises a polymer coating.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon; wherein the pharmaceutical tablet comprises about 177.24 mg of microcrystalline cellulose; about 95.76 mg of lactose monohydrate; about 9 mg sodium starch glycolate; and about 3 mg magnesium stearate; wherein a coating is added to the pharmaceutical tablet; wherein the coating comprises a polymer coating.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon; wherein the pharmaceutical tablet comprises about 177.24 mg of microcrystalline cellulose; about 95.76 mg of lactose monohydrate; about 9 mg sodium starch glycolate; and about 3 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 15 mg of tavapadon; wherein the pharmaceutical tablet comprises about 177.24 mg of microcrystalline cellulose; about 95.76 mg of lactose monohydrate; about 9 mg sodium starch glycolate; and about 3 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon; wherein the pharmaceutical tablet comprises about 118.16 mg of microcrystalline cellulose; about 63.84 mg of lactose monohydrate; about 6 mg sodium starch glycolate; and about 2 mg magnesium stearate.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon; wherein the pharmaceutical tablet comprises about 118.16 mg of microcrystalline cellulose; about 63.84 mg of lactose monohydrate; about 6 mg sodium starch glycolate; and about 2 mg magnesium stearate.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon; wherein the pharmaceutical tablet comprises about 118.16 mg of microcrystalline cellulose; about 63.84 mg of lactose monohydrate; about 6 mg sodium starch glycolate; and about 2 mg magnesium stearate; wherein a coating is added to the pharmaceutical tablet.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon; wherein the pharmaceutical tablet comprises about 118.16 mg of microcrystalline cellulose; about 63.84 mg of lactose monohydrate; about 6 mg sodium starch glycolate; and about 2 mg magnesium stearate; wherein a coating is added to the pharmaceutical tablet.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon; wherein the pharmaceutical tablet comprises about 118.16 mg of microcrystalline cellulose; about 63.84 mg of lactose monohydrate; about 6 mg sodium starch glycolate; and about 2 mg magnesium stearate; wherein a coating is added to the pharmaceutical tablet; wherein the coating comprises a polymer coating.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon; wherein the pharmaceutical tablet comprises about 118.16 mg of microcrystalline cellulose; about 63.84 mg of lactose monohydrate; about 6 mg sodium starch glycolate; and about 2 mg magnesium stearate; wherein a coating is added to the pharmaceutical tablet; wherein the coating comprises a polymer coating.

One aspect of the invention pertains to a pharmaceutical tablet comprising: about 0.25% by weight to about 15% by weight of tavapadon free base; and at least about 80% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon; wherein the pharmaceutical tablet comprises about 118.16 mg of microcrystalline cellulose; about 63.84 mg of lactose monohydrate; about 6 mg sodium starch glycolate; and about 2 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

One aspect of the invention pertains to a pharmaceutical tablet comprising: about 0.25% by weight to about 5% by weight of tavapadon free base; and at least about 90% by weight of at least one filler; further comprising: at least one disintegrant; and at least one lubricant; wherein: at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof, at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof; wherein at least one disintegrant is present in an amount of about 3% by weight and at least one lubricant is present in an amount of about 1% by weight; wherein: at least one filler comprises lactose and cellulose; at least one disintegrant is a starch; and at least one lubricant is stearic acid or salts thereof; wherein: at least one filler comprises lactose monohydrate and microcrystalline cellulose; at least one disintegrant is sodium starch glycolate; and at least one lubricant is magnesium stearate comprising: about 5% by weight of tavapadon; about 59% by weight of microcrystalline cellulose; about 32% by weight of lactose monohydrate; about 3% sodium starch glycolate; and about 1% magnesium stearate; wherein the pharmaceutical tablet comprises about 10 mg of tavapadon; wherein the pharmaceutical tablet comprises about 118.16 mg of microcrystalline cellulose; about 63.84 mg of lactose monohydrate; about 6 mg sodium starch glycolate; and about 2 mg magnesium stearate; wherein a coating is added to the pharmaceutical tablet; wherein the coating comprises a polymer coating; wherein the coating comprises polyvinyl alcohol, polyethylene glycol, titanium dioxide, and talc.

Another aspect of the invention pertains to any of the preceding embodiments wherein the pharmaceutical tablet comprises about 0.25 mg of tavapadon unless otherwise stated.

Another aspect of the invention pertains to any of the preceding embodiments wherein the pharmaceutical tablet comprises about 1 mg of tavapadon unless otherwise stated.

Another aspect of the invention pertains to any of the preceding embodiments wherein the pharmaceutical tablet comprises about 5 mg of tavapadon unless otherwise stated.

Another aspect of the invention pertains to any of the preceding embodiments wherein the pharmaceutical tablet comprises about 10 mg of tavapadon unless otherwise stated.

Another aspect of the invention pertains to any of the preceding embodiments wherein the pharmaceutical tablet comprises about 15 mg of tavapadon unless otherwise stated.

Another aspect of the invention pertains to a pharmaceutical tablet comprising tavapadon and at least one filler; wherein the tavapadon is comprised of particles having an average D[4,3] of about 12 μm.

Another aspect of the invention pertains to a pharmaceutical tablet comprising tavapadon and at least one filler; wherein the tavapadon is comprised of particles having an average D[4,3] of about 12 μm; wherein the pharmaceutical tablet comprises about 0.25 mg of tavapadon.

Another aspect of the invention pertains to a pharmaceutical tablet comprising tavapadon and at least one filler; wherein the tavapadon is comprised of particles having an average D[4,3] of about 12 μm; wherein the pharmaceutical tablet comprises about 1 mg of tavapadon.

Another aspect of the invention pertains to a pharmaceutical tablet comprising tavapadon and at least one filler; wherein the tavapadon is comprised of particles having an average D[4,3] of about 12 μm; wherein the pharmaceutical tablet comprises about 5 mg of tavapadon.

Another aspect of the invention pertains to a pharmaceutical tablet comprising tavapadon and at least one filler; wherein the tavapadon is comprised of particles having an average D[4,3] of about 12 μm; wherein the pharmaceutical tablet comprises about 10 mg of tavapadon.

Another aspect of the invention pertains to a pharmaceutical tablet comprising tavapadon and at least one filler; wherein the tavapadon is comprised of particles having an average D[4,3] of about 12 μm; wherein the pharmaceutical tablet comprises about 15 mg of tavapadon.

Another aspect of the invention pertains to a pharmaceutical tablet comprising tavapadon and at least one filler, wherein about 75% or more of the tavapadon is released in 20 minutes in 0.01N HCl with 0.15% or 0.2% SDS.

Another aspect of the invention pertains to a pharmaceutical tablet comprising tavapadon and at least one filler, wherein about 75% or more of the tavapadon is released in 20 minutes in 0.01N HCl with 0.15% or 0.2% SDS; wherein the pharmaceutical tablet comprises about 0.25 mg of tavapadon.

Another aspect of the invention pertains to a pharmaceutical tablet comprising tavapadon and at least one filler, wherein about 75% or more of the tavapadon is released in 20 minutes in 0.01N HCl with 0.15% or 0.2% SDS; wherein the pharmaceutical tablet comprises about 1 mg of tavapadon.

Another aspect of the invention pertains to a pharmaceutical tablet comprising tavapadon and at least one filler, wherein about 75% or more of the tavapadon is released in 20 minutes in 0.01N HCl with 0.15% or 0.2% SDS; wherein the pharmaceutical tablet comprises about 5 mg of tavapadon.

Another aspect of the invention pertains to a pharmaceutical tablet comprising tavapadon and at least one filler, wherein about 75% or more of the tavapadon is released in 20 minutes in 0.01N HCl with 0.15% or 0.2% SDS; wherein the pharmaceutical tablet comprises about 10 mg of tavapadon.

Another aspect of the invention pertains to a pharmaceutical tablet comprising tavapadon and at least one filler, wherein about 75% or more of the tavapadon is released in 20 minutes in 0.01N HCl with 0.15% or 0.2% SDS; wherein the pharmaceutical tablet comprises about 15 mg of tavapadon.

Another embodiment pertains to a method of manufacturing a pharmaceutical tablet comprising tavapadon and at least one filler; the steps comprising:
(a) blending tavapadon with filler to obtain a pre-compaction blend;
(b) compacting the pre-compaction blend to form ribbons;
(c) milling the ribbons into granules; and
(d) compressing into tablets.

Another embodiment pertains to a method of manufacturing a pharmaceutical tablet comprising tavapadon and at least one filler; the steps comprising:
(a) blending tavapadon with filler and disintegrant to obtain a pre-compaction blend;
(b) compacting the pre-compaction blend to form ribbons;
(c) milling the ribbons into granules; and
(d) compressing into tablets.

Another embodiment pertains to a method of manufacturing a pharmaceutical tablet comprising tavapadon and at least one filler; the steps comprising:
(a) blending tavapadon with filler, disintegrant and intragranular portion lubricant to obtain a pre-compaction blend;
(b) compacting the pre-compaction blend to form ribbons;
(c) milling the ribbons into granules; and
(d) compressing into tablets.

Another embodiment pertains to a method of manufacturing a pharmaceutical tablet, the steps comprising
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets.

Another embodiment pertains to a method of manufacturing a pharmaceutical tablet, the steps comprising
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets, wherein the disintegrant is sodium starch glycolate.

Another embodiment pertains to a method of manufacturing a pharmaceutical tablet, the steps comprising
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets, wherein the disintegrant is sodium starch glycolate and the lubricant is magnesium stearate.

Another embodiment pertains to a method of manufacturing a pharmaceutical tablet, the steps comprising
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets, wherein the disintegrant is sodium starch glycolate, the lubricant is magnesium stearate, and the filler is microcrystalline cellulose and lactose monohydrate.

Another embodiment pertains to a pharmaceutical tablet comprising tavapadon and at least one filler wherein disintegration of the tablet is achieved within 15 minutes under guidelines and requirements of United States Pharmacopeia (USP) general chapter <701>.

Another embodiment pertains to a pharmaceutical tablet comprising tavapadon and at least one filler, manufactured by:
(a) blending tavapadon with filler to obtain a pre-compaction blend;
(b) compacting the pre-compaction blend to form ribbons;
(c) milling the ribbons into granules; and
(d) compressing into tablets.

Another embodiment pertains to a pharmaceutical tablet comprising tavapadon and at least one filler, manufactured by:
(a) blending tavapadon with filler and disintegrant to obtain a pre-compaction blend; (b) compacting the pre-compaction blend to form ribbons;
(c) milling the ribbons into granules; and
(d) compressing into tablets.

Another embodiment pertains to a pharmaceutical tablet comprising tavapadon and at least one filler, manufactured by:
(a) blending tavapadon with filler, disintegrant, and intragranular portion lubricant to obtain a pre-compaction blend;
(b) compacting the pre-compaction blend to form ribbons;
(c) milling the ribbons into granules; and
(d) compressing into tablets.

Another embodiment pertains to a pharmaceutical tablet comprising tavapadon and at least one filler; manufactured by
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets.

Another embodiment pertains to a pharmaceutical tablet comprising tavapadon and at least one filler; manufactured by
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets; wherein the disintegrant is sodium starch glycolate.

Another embodiment pertains to a pharmaceutical tablet comprising tavapadon and at least one filler; manufactured by
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets; wherein the disintegrant is sodium starch glycolate and the lubricant is magnesium stearate.

Another embodiment pertains to a pharmaceutical tablet comprising tavapadon and at least one filler; manufactured by
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets; wherein the disintegrant is sodium starch glycolate, the lubricant is magnesium stearate, and the filler is microcrystalline cellulose and lactose monohydrate.

Another embodiment pertains to pharmaceutical tablet comprising tavapadon and at least one filler, wherein 90% or more dissolution of the tablet is achieved under guidelines and requirements of United States Pharmacopeia (USP) general chapter <711>.

Another embodiment pertains to a method for treating Parkinson's Disease (PD) in a patient, comprising administering to the patient a pharmaceutical formulation comprising:
(a) about 0.25% by weight to about 15% by weight of tavapadon free base; and
(b) at least about 80% by weight of at least one filler; wherein the Parkinson's Disease is effectively treated.

Another embodiment pertains to a pharmaceutical tablet comprising tavapadon and at least one filler; wherein the tavapadon is comprised of particles having an average D[4,3] of about <25 μm.

Another embodiment pertains to a pharmaceutical tablet comprising tavapadon and at least one filler; wherein the tavapadon is comprised of particles having an average D[4,3] of about <25 μm; wherein the pharmaceutical tablet comprises about 0.25 mg of tavapadon.

Another embodiment pertains to a pharmaceutical tablet comprising tavapadon and at least one filler; wherein the tavapadon is comprised of particles having an average D[4,3] of about <25 μm; wherein the pharmaceutical tablet comprises about 1 mg of tavapadon.

Another embodiment pertains to a pharmaceutical tablet comprising tavapadon and at least one filler; wherein the tavapadon is comprised of particles having an average D[4,3] of about <25 μm; wherein the pharmaceutical tablet comprises about 5 mg of tavapadon.

Another embodiment pertains to a pharmaceutical tablet comprising tavapadon and at least one filler; wherein the tavapadon is comprised of particles having an average D[4,3] of about <25 μm; wherein the pharmaceutical tablet comprises about 10 mg of tavapadon.

Another embodiment pertains to a pharmaceutical tablet comprising tavapadon and at least one filler; wherein the tavapadon is comprised of particles having an average D[4,3] of about <25 μm; wherein the pharmaceutical tablet comprises about 15 mg of tavapadon.

### BRIEF DESCRIPTION OF THE DRAWINGS

FIG. 1: Process Flow for the Manufacturing of Tavapadon 0.25 mg Tablets Used in Phase 3 Clinical Studies

FIG. 2: Process Flow for the Manufacturing of Tavapadon 1 and 5 mg Tablets Used in Phase 3 Clinical Studies

FIG. 3: Dissolution Profile for Varying API Particle Size (0.25 mg)

FIG. 4: Dissolution Profile for Varying API Particle Size (1 mg)

FIG. 5: Dissolution Profile for Varying API Particle Size (5 mg)

FIG. 6: Dissolution Profile for Varying API Particle Size (15 mg)

FIG. 7: Varying Levels of Disintegrant, Sodium Starch Glycolate, 0.25 mg

FIG. 8: Varying Levels of Disintegrant, Sodium Starch Glycolate, 1 mg

FIG. 9: Varying Levels of Disintegrant, Sodium Starch Glycolate, 15 mg

FIG. 10: 0.25 mg Core vs. Coated

FIG. 11: 1 mg Core vs. Coated

FIG. 12: 5 mg Core vs. Coated

FIG. 13: 10 mg Core vs. Coated

FIG. 14: 15 mg Core vs. Coated

FIG. 15. Aqueous Dissolution for 0.25 mg

FIG. 16. Aqueous Dissolution for 1 mg

FIG. 17. 1 mg Apparatus and RPM Evaluation in 900 mL of 0.01N HCl with 0.15% SDS

FIG. 18. 15 mg Apparatus and RPM Evaluation in 900 mL of 0.01N HCl with 0.2% SDS

FIG. 19. Media Evaluation for 0.25 mg Tablets in 900 mL at 75 RPM

FIG. 20. Media Evaluation for 1 mg Tablets in 900 mL at 75 RPM

FIG. 21. Media Evaluation for 15 mg Tablets in 900 mL at 75 RPM

FIG. 22. Media Evaluation for 5 mg, 10 mg, and 15 mg Tablets in 900 mL at 75 RPM

FIG. 23. Biorelevant 0.25 mg

FIG. 24. Biorelevant 1 mg

FIG. 25. Biorelevant 15 mg

FIG. 26. 0.25 mg Coated vs. Uncoated

FIG. 27. 1 mg Coated vs. Uncoated

FIG. 28. 5 mg Coated vs. Uncoated

FIG. 29. 10 mg Coated vs. Uncoated

FIG. 30. 15 mg Coated vs. Uncoated

FIG. 31. 0.25 mg Particle Size

FIG. 32. 1 mg Particle Size

FIG. 33. 5 mg Particle Size

FIG. 34. 15 mg Particle Size

FIG. 35. Formulations Evaluation, Sodium Starch Glycolate, 0.25 mg

FIG. 36. Formulation Evaluation, Sodium Starch Glycolate, 1 mg

FIG. 37. Formulation Evaluation, Sodium Starch Glycolate, 15 mg

FIG. 38. Manufacturing Process Flow for Tavapadon 0.25 mg Tablets

FIG. 39. Manufacturing Process Flow for Tavapadon 1.0 mg and 5.0 mg Tablets

FIG. 40. Manufacturing Process Flow for 15 mg Tavapadon Tablets

FIG. 41. Dissolution of 15 mg Tavapadon Tablets

### DETAILED DESCRIPTION

The present invention provides for pharmaceutical tablets comprising tavapadon (e.g., tavapadon free base) and processes for preparing such dosage forms.

### Drug Substance

As used herein, the term “tavapadon” refers to the compound with the International Nonproprietary Name (INN) of tavapadon, also referred to as (S)-(−)-1,5-dimethyl-6-[2-methyl-4-[[3-(trifluoromethyl)-2-pyridinyl]oxy]phenyl]-2,4(1H,3H)-pyrimidinedione and shown as Compound 1.

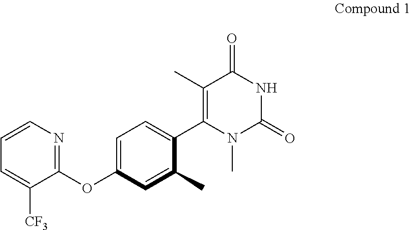

In some embodiments, the pharmaceutical tablet comprises the free base form of tavapadon.

In some embodiments, the pharmaceutical tablet comprises about about 15% by weight of tavapadon. In some embodiments, the pharmaceutical tablet comprises about 0.25% by weight to about 5% by weight of tavapadon, such as about 0.25% by weight, about 1% by weight, or about 5% by weight of tavapadon. In some embodiments, the pharmaceutical tablet comprises tavapadon free base.

In some embodiments, the pharmaceutical tablet comprises about 0.25 mg to about 15 mg of tavapadon, such as about 0.25 mg, about 1 mg, about 5 mg, about 10 mg or about 15 mg of tavapadon.

### Formulation

In some embodiments, pharmaceutical tablets comprise tavapadon and one or more excipients. In some embodiments, pharmaceutical tablets comprise tavapadon and one or more fillers, disintegrants, and/or lubricants. It should be noted that many excipients may have more than one function in a pharmaceutical composition. As such, characterization herein of a particular excipient as having a certain function, e.g., filler, disintegrant, lubricant, etc., should not be read as limiting to that function.

Fillers selected preferably exhibit suitable flow properties and, where tablets are desired, compressibility. Suitable fillers include, either individually or in combination, lactose, including anhydrous lactose and lactose monohydrate; lactitol; maltitol; mannitol; sorbitol; xylitol; dextrose and dextrose monohydrate; fructose; sucrose and sucrose-based diluents, such as compressible sugar, confectioner's sugar, and sugar spheres; maltose; inositol; hydrolyzed cereal solids; starches (e.g., corn starch, wheat starch, rice starch, potato starch, tapioca starch, etc.), starch components such as amylose and dextrates, and modified or processed starches such as pregelatinized starch; dextrins; celluloses including powdered cellulose, microcrystalline cellulose, silicified microcrystalline cellulose, food grade sources of α- and amorphous cellulose and powdered cellulose, and cellulose acetate; calcium salts including calcium carbonate, tribasic calcium phosphate, dicalcium phosphate (e.g., dibasic calcium phosphate), monobasic calcium sulfate monohydrate, calcium sulfate and granular calcium lactate trihydrate; magnesium carbonate; magnesium oxide; bentonite; kaolin; sodium chloride; and the like.

In embodiments, the filler of the pharmaceutical tablet comprises cellulose, such as microcrystalline cellulose. In embodiments, the filler of the pharmaceutical tablet comprises cellulose, such as silicified microcrystalline cellulose. In embodiments, the filler of the pharmaceutical tablet comprises dibasic calcium phosphate. In embodiments, the filler of the pharmaceutical tablet comprises mannitol. In embodiments, the filler of the pharmaceutical tablet comprises pregelatinized starch. In some embodiments, the filler of the pharmaceutical tablet comprises lactose, such as lactose monohydrate. In some embodiments, the filler of the pharmaceutical tablet comprises cellulose and dibasic calcium phosphate. In some embodiments, the filler of the pharmaceutical tablet comprises cellulose and lactose, such as microcrystalline cellulose and lactose monohydrate. In some embodiments, the filler of the pharmaceutical tablet comprises cellulose, such as microcrystalline cellulose and mannitol. In some embodiments, the filler of the pharmaceutical tablet comprises cellulose, such as microcrystalline cellulose and pregelatinized starch. In some embodiments, the filler of the pharmaceutical tablet comprises lactose, such as lactose monohydrate, and pregelatinized starch. In embodiments, the filler of the pharmaceutical tablet comprises cellulose, such as silicified microcrystalline cellulose and pregelatinized starch. In embodiments, the filler of the pharmaceutical tablet comprises mannitol and pregelatinized starch.

Such fillers, if present, may constitute at least about 80% by weight of the pharmaceutical tablet, such as from about 80% to about 96% by weight of the pharmaceutical tablet, or about 90% to about 96% by weight of the pharmaceutical tablet, or about 81%, about 90%, about 91%, about 95% or about 96% by weight of the pharmaceutical tablet. For example, a pharmaceutical tablet with a core tablet total weight of 300 mg may comprise from about 240 mg to about 288 mg of filler, or from about 265 mg to about 280 mg of filler, such as 265 mg, 270 mg, 273, or 275 mg of filler. For example, a pharmaceutical tablet with a core tablet total weight of 100 mg may comprise from about 81 to about 96 mg of filler, or about 81 mg, 91 mg, 95 mg, 95.5 mg, or 95.75 mg of filler.

In some embodiments, the pharmaceutical tablet may comprise at least two fillers. In such embodiments, the first filler may constitute from about 40% to about 50% by weight of the pharmaceutical tablet, such as about 43%, about 45%, or about 46% by weight of the pharmaceutical tablet. For example, a pharmaceutical tablet with a core tablet total weight of 300 mg may comprise from about 120 mg to about 150 mg of the first filler, such as 136.5 mg of filler. The second filler may constitute from about 40% to about 50% by weight of the pharmaceutical tablet, such as 43% or about 45% or about 46% by weight of the pharmaceutical tablet. For example, a pharmaceutical tablet with a core tablet total weight of 300 mg may comprise from about 120 mg to about 150 mg of the second filler, such as about 136.5 mg of filler.

In such embodiments, the first filler may constitute from about 52% to about 56% by weight of the pharmaceutical tablet, such as about 53%, about 54%, or about 55% by weight of the pharmaceutical tablet. For example, a pharmaceutical tablet with a core tablet total weight of 100 mg may comprise from about 52 mg to about 56 mg of the first filler, such as 54.08 mg of filler. The second filler may constitute from about 24% to about 28% by weight of the pharmaceutical tablet, such as about 26% or about 27% by weight of the pharmaceutical tablet. For example, a pharmaceutical tablet with a core tablet total weight of 100 mg may comprise from about 26 mg to about 27 mg of the second filler, such as about 26.92 mg of filler.

In such embodiments, the first filler may constitute from about 58% to about 64% by weight of the pharmaceutical tablet, such as about 59%, about 63%, or about 64% by weight of the pharmaceutical tablet. For example, a pharmaceutical tablet with a core tablet total weight of 300 mg may comprise from about 175 mg to about 180 mg of the first filler, such as 177 mg of filler. The second filler may constitute from about 31% to about 33% by weight of the pharmaceutical tablet, such as 31% or 32% by weight of the pharmaceutical tablet. For example, a pharmaceutical tablet with a core tablet total weight of 300 mg may comprise from about 93 mg to about 99 mg of the second filler, such as about 96 mg of filler.

In embodiments, the first filler comprises cellulose, such as microcrystalline cellulose. In embodiments, the first filler comprises cellulose, such as silicified microcrystalline cellulose. In embodiments, the first filler comprises mannitol. In some embodiments, the first filler comprises pregelatinized starch. In some embodiments, the second filler comprises mannitol. In some embodiments, the second filler comprises pregelatinized starch. In some embodiments, the second filler comprises dibasic calcium phosphate. In some embodiments, the second filler comprises lactose, such as lactose monohydrate. In some embodiments, the first filler comprises cellulose and the second filler comprises dibasic calcium phosphate. In some embodiments, the first filler comprises cellulose and the second filler comprises lactose, such as microcrystalline cellulose and lactose monohydrate. In some embodiments, the filler comprises cellulose, such as microcrystalline cellulose. In some embodiments, the first filler comprises cellulose, such as microcrystalline cellulose and the second filler comprises mannitol. In some embodiments, the first filler comprises cellulose, such as microcrystalline cellulose and the second filler comprises pregelatinized starch. In some embodiments, the first filler comprises pregelatinized starch and the second filler comprises lactose monohydrate. In some embodiments, the filler comprises mannitol. In some embodiments, the filler comprises pregelatinized starch. In embodiments, the first filler comprises cellulose, such as silicified microcrystalline cellulse and the second filler comprises pregelatinized starch. In embodiments, the first filler comprises mannitol and the second filler comprises pregelatinized starch.

Suitable disintegrants include, either individually or in combination, starches including pregelatinized starch and sodium starch glycolate; clays; magnesium aluminum silicate; cellulose-based disintegrants such as powdered cellulose, microcrystalline cellulose, methylcellulose, low-substituted hydroxypropylcellulose, carmellose, carmellose calcium, carmellose sodium and croscarmellose sodium; alginates; povidone; crospovidone; polacrilin potassium; gums such as agar, guar, locust bean, karaya, pectin and tragacanth gums; colloidal silicon dioxide; and the like. In embodiments, the disintegrant comprises sodium starch glycolate.

Such disintegrants, if present, may constitute about 3% by weight of the pharmaceutical tablet. For example, a pharmaceutical tablet with a core tablet total weight of 300 mg may comprise about 9 mg of disintegrant. For example, a pharmaceutical tablet with a core tablet total weight of 100 mg may comprise about 3 mg of disintegrant.

Lubricants reduce friction between a tableting mixture and tableting equipment during compression of tablet formulations. Suitable lubricants include, either individually or in combination, glyceryl behenate; stearic acid and salts thereof, including magnesium, calcium and sodium stearates; hydrogenated vegetable oils; glyceryl palmitostearate; talc; waxes; sodium benzoate; sodium acetate; sodium fumarate; sodium stearyl fumarate; PEGs (e.g., PEG 4000 and PEG 6000); poloxamers; polyvinyl alcohol; sodium oleate; sodium lauryl sulfate; magnesium lauryl sulfate; and the like. In some embodiments, the lubricant comprises magnesium stearate.

Such lubricants, if present, may constitute about 1% by weight of the pharmaceutical tablet. For example, a pharmaceutical tablet with a core tablet total weight of 300 mg may comprise about 3 mg of lubricant. For example, a pharmaceutical tablet with a core tablet total weight of 100 mg may comprise about 1 mg of lubricant.

If present, the pharmaceutical tablet may comprise one or more lubricants in the intra-granular phase, the extra-granular phase, or both. In some embodiments, the intra-granular lubricant(s) may constitute about 0.5% by weight of the pharmaceutical tablet. For example, a pharmaceutical tablet with a core tablet with a total weight of 500 mg may comprise about 2.5 mg of lubricant in the intra-granular phase. For example, a pharmaceutical tablet with a core tablet with a total weight of 300 mg may comprise about 1.5 mg of lubricant in the intra-granular phase. For example, a pharmaceutical tablet with a core tablet with a total weight of 100 mg may comprise about 0.5 mg of lubricant in the intra-granular phase. In some embodiments, the extra-granular lubricant(s) may constitute about 0.5% by weight of the pharmaceutical tablet. For example, a pharmaceutical tablet with a core tablet total weight of 500 mg may comprise about 2.5 mg of lubricant in the extra-granular phase. For example, a pharmaceutical tablet with a core tablet total weight of 300 mg may comprise about 1.5 mg of lubricant in the extra-granular phase. For example, a pharmaceutical tablet with a core tablet total weight of 100 mg may comprise about 0.5 mg of lubricant in the extra-granular phase.

In embodiments, a coating is added to the pharmaceutical tablet. In some embodiments, the coating comprises a polymer coating. The coating may make the compressed material smoother and easier to swallow, can make the material more resistant to the environment (extending shelf life), and/or can enhance the tablet's appearance. In some embodiments, the coating may comprise one or more of polyvinyl alcohol, which may be partially hydrolyzed, polyethylene glycol, titanium dioxide, and talc. In some embodiments, the coating may further comprise one or more colorants. For example, in embodiments, the coating may comprise Opadry® II 85F100132 Purple or Opadry II 85F130189 Orange.

In some embodiments, the pharmaceutical tablet comprises tavapadon and one or more excipients. In some embodiments, the one or more excipients comprises one or more fillers, disintegrants, and/or lubricants. In some embodiments, the one or more excipients comprises one or more fillers. In some embodiments, the one or more excipients comprises one or more disintegrants. In some embodiments, the one or more excipients comprises one or more lubricants. In some embodiments, the one or more excipients comprises one or more fillers and one or more disintegrants. In some embodiments, the one or more excipients comprises one or more fillers and one or more lubricants. In some embodiments, the one or more excipients comprises one or more fillers, one or more lubricants, and one or more disintegrants. Each of tavapadon, the one or more fillers, the one or more lubricants, and the one or more disintegrants may be included in the forms and amounts disclosed above.

In some embodiments the pharmaceutical tablet comprises one or more crystalline forms of tavapadon. In some embodiments, one or more crystalline forms of tavapadon are those described in PCT Patent Publication No. WO 2023/102087, which is incorporated by reference in its entirety.

Immediate release uncoated tablet formulations of 0.25 mg, 1.0 mg, 5.0 mg, 10 mg, and 15 mg doses have been developed for tavapadon. The formulation consists of the tavapadon drug substance and the following excipients: microcrystalline cellulose (filler), lactose monohydrate (filler), sodium starch glycolate (disintegrant), and magnesium stearate (lubricant). Depending upon the scale and strength, the tablets have been manufactured using either direct compression or dry granulation process to ensure content uniformity and manufacturability.

The initial uncoated tablet formulation for the 1 mg and 5 mg tablets were manufactured in a formulation that contains anhydrous dibasic calcium phosphate (DCP) as a filler. During the ongoing formulation and process development, it was found that replacing DCP with lactose monohydrate, an alternative filler, provided improved chemical stability of tavapadon tablets.

A subsequent film coated tablet formulation was developed for 15 mg tablets and it is the same formulation used in Phase 3 studies except for the film coating system. This film coated tablet formulation will be used in the bioequivalence study to support the introduction of 15 mg tablets. The qualitative formulation consists of the tavapadon drug substance and the following excipients: microcrystalline cellulose (filler), lactose monohydrate (filler), sodium starch glycolate (disintegrant), magnesium stearate (lubricant) and a film coating system. The manufacturing process involves blending the drug substance, microcrystalline cellulose, lactose monohydrate, sodium starch glycolate, and intragranular portion magnesium stearate to form pre-compaction blend. The pre-compaction blend is roller compacted to form ribbons and then milled into granules. The granules are then blended with extragranular portion magnesium stearate to form final blend and compressed into tablets. The tablets are film coated to form the final image.

TABLE 1
Composition of Tavapadon 0.25 mg Tablet

| | | Unit | |
|---|---|---|---|
| | | Formula | Amount |
| Name of Ingredients | Function | (mg/tablet) | (% w/w) |
| Tavapadon^{a} | Active | 0.25 | 0.25 |
| | Ingredient | | |
| Microcrystalline | Filler | 63.83 | 63.83 |
| Cellulose^{b} | | | |
| Lactose Monohydrate | Filler | 31.92 | 31.92 |
| Sodium Starch | Disintegrant | 3.00 | 3.00 |
| Glycolate | | | |
| Magnesium Stearate | Lubricant | 1.00 | 1.00 |
| (Non-Bovine) | | | |
| Total Tablet Weight | | 100.00 mg | |

^{a}Based on theoretical potency of 1.000. Batch quantity may be adjusted based on the actual potency.
^{b}Quantity of Microcrystalline Cellulose may be adjusted for slight potency changes of tavapadon to ensure tablet weight.

TABLE 2
Composition of Tavapadon 1.0 mg Tablet

| | | Unit | |
|---|---|---|---|
| | | Formula | Amount |
| Name of Ingredients | Function | (mg/tablet) | (% w/w) |
| Tavapadon^{a} | Active | 1.00 | 1.00 |
| | Ingredient | | |
| Microcrystalline | Filler | 63.08 | 63.08 |
| Cellulose^{b} | | | |
| Lactose Monohydrate | Filler | 31.92 | 31.92 |
| Sodium Starch | Disintegrant | 3.00 | 3.00 |
| Glycolate | | | |
| Magnesium Stearate | Lubricant | 1.00 | 1.00 |
| (Non-Bovine) | | | |
| Total Tablet Weight | | 100.00 mg | |

^{a}Based on theoretical potency of 1.000. Batch quantity may be adjusted based on the actual potency.
^{b}Quantity of Microcrystalline Cellulose may be adjusted for slight potency changes of tavapadon to ensure tablet weight.

TABLE 3
Composition of Tavapadon 5.0 mg Tablet

| | | Unit | |
|---|---|---|---|
| | | Formula | Amount |
| Name of Ingredients | Function | (mg/tablet) | (% w/w) |
| Tavapadon^{a} | Active | 5.00 | 5.00 |
| | Ingredient | | |
| Microcrystalline | Filler | 59.08 | 59.08 |
| Cellulose^{b} | | | |
| Lactose Monohydrate | Filler | 31.92 | 31.92 |
| Sodium Starch | Disintegrant | 3.00 | 3.00 |
| Glycolate | | | |
| Magnesium Stearate | Lubricant | 1.00 | 1.00 |
| (Non-Bovine) | | | |
| Total Tablet Weight | | 100.00 mg | |

^{a}Based on theoretical potency of 1.000. Batch quantity may be adjusted based on the actual potency.
^{b}Quantity of Microcrystalline Cellulose may be adjusted for slight potency changes of tavapadon to ensure tablet weight.

TABLE 4
Composition of Tavapadon 15.0 mg Tablet

| | | Unit | |
|---|---|---|---|
| | | Formula | Amount |
| Name of Ingredients | Function | (mg/tablet) | (% w/w) |
| Tavapadon^{a} | Active | 15.00 | 5.00 |
| | Ingredient | | |
| Microcrystalline | Filler | 177.24 | 59.08 |
| Cellulose^{b} | | | |
| Lactose Monohydrate | Filler | 95.76 | 31.92 |
| Sodium Starch | Disintegrant | 9.00 | 3.00 |
| Glycolate | | | |
| Magnesium Stearate | Lubricant | 3.00 | 1.00 |
| (Non-Bovine) | | | |
| Total Tablet Weight | | 300.00 mg | |

^{a}Based on theoretical potency of 1.000. Batch quantity may be adjusted based on the actual potency.
^{b}Quantity of Microcrystalline Cellulose may be adjusted for slight potency changes of tavapadon to ensure tablet weight.

TABLE 5A
Components and Composition of Coated Tavapadon Tablets (0.25 mg)

| | Target | | |
|---|---|---|---|
| | Quantity Per | Amount | |
| Component | Tablet (mg) | (wt %) | Function |
| Tavapadon | 0.25 | 0.25 | Active |
| | | | Ingredient |
| Microcrystalline | 63.83 | 63.83 | Diluent |
| Cellulose | | | |
| Lactose Monohydrate | 31.92 | 31.92 | Diluent |
| Sodium Starch | 3.00 | 3.00 | Disintegrant |
| Glycolate | | | |
| Magnesium Stearate | 1.00 | 1.00 | Lubricant |
| Core Tablet Total | 100.00 | 100.00 | |
| Film Coat | | | |
| OPADRY ® II Orange¹ | 4.00 | 4.00 | Coating Agent |
| Purified Water² | — | — | Processing Aid |
| Total Coated Tablet | 104.00 | 104.00 | |

¹Refer to Table 5C for the components and composition of the excipient, OPADRY ® II Orange 85F130189.
²Removed during processing.

TABLE 5B
Components and Composition of Coated Tavapadon Tablets (1 mg)

| | | | |
|---|---|---|---|
| | Target | | |
| | Quantity Per | Amount | |
| Component | Tablet (mg) | (wt %) | Function |

Intragranular Components

| | | | |
|---|---|---|---|
| Tavapadon | 1.00 | 1.00 | Active Ingredient |
| Microcrystalline | 63.08 | 63.08 | Diluent |
| Cellulose | | | |
| Lactose Monohydrate | 31.92 | 31.92 | Diluent |
| Sodium Starch | 3.00 | 3.00 | Disintegrant |
| Glycolate | | | |
| Magnesium Stearate | 0.50 | 0.50 | Lubricant |

Extragranular Components

| | | | |
|---|---|---|---|
| Magnesium Stearate | 0.50 | 0.50 | Lubricant |
| Core Tablet Total | 100.00 | 100.00 | |

Film Coat

| | | | |
|---|---|---|---|
| OPADRY ® II Orange¹ | 4.00 | 4.00 | Coating Agent |
| Purified Water² | — | — | Processing Aid |
| Total Coated Tablet | 104.00 | 104.00 | |

¹Refer to Table 5C for the components and composition of the excipient, OPADRY ® II Orange 85F130189.
²Removed during processing.

TABLE 5C
Components and Composition of OPADRY ® II Orange 85F130189

| Component | Quality Standard | Composition (% w/w) |
|---|---|---|
| Polyvinyl Alcohol | USP | 40.00 |
| Titanium Dioxide | USP | 21.73 |
| Macrogol | USP | 20.20 |
| Talc | USP | 14.80 |
| Iron Oxide Yellow | NF | 2.54 |
| Iron Oxide Red | NF | 0.73 |

TABLE 5D
Components and Composition of Coated Tavapadon Tablets (5, 10 and 15 mg)

| | Target Quantity Per | | | | | | |
|---|---|---|---|---|---|---|---|
| | Tablet (mg) | | | Amount (wt %) | | | |
| Component | 5 mg | 10 mg | 15 mg | 5 mg | 10 mg | 15 mg | Function |

Intragranular Components

| | | | | | | | |
|---|---|---|---|---|---|---|---|
| Tavapadon | 5.00 | 10.00 | 15.00 | 5.00 | 5.00 | 5.00 | Active |
| | | | | | | | Ingredient |
| Microcrystalline | 59.08 | 118.16 | 177.24 | 59.08 | 59.08 | 59.08 | Diluent |
| Cellulose | | | | | | | |
| Lactose | 31.92 | 63.84 | 95.76 | 31.92 | 31.92 | 31.92 | Diluent |
| Monohydrate | | | | | | | |
| Sodium Starch | 3.00 | 6.00 | 9.00 | 3.00 | 3.00 | 3.00 | Disintegrant |
| Glycolate | | | | | | | |
| Magnesium | 0.50 | 1.00 | 1.50 | 0.50 | 0.50 | 0.50 | Lubricant |
| Stearate | | | | | | | |

Extragranular Components

| | | | | | | | |
|---|---|---|---|---|---|---|---|
| Magnesium | 0.50 | 1.00 | 1.50 | 0.50 | 0.50 | 0.50 | Lubricant |
| Stearate | | | | | | | |
| Core Tablet | 100.00 | 200.00 | 300.00 | 100.00 | 100.00 | 100.00 | |
| Total | | | | | | | |

Film Coat

| | | | | | | | |
|---|---|---|---|---|---|---|---|
| OPADRY ® II | 4.00 | 8.00 | 12.00 | 4.00 | 4.00 | 4.00 | Coating |
| Purple¹ | | | | | | | Agent |
| Purified Water² | — | — | — | — | — | — | Processing |
| | | | | | | | Aid |
| Total Coated | 104.00 | 208.00 | 312.00 | 104.00 | 104.00 | 104.00 | |
| Tablet | | | | | | | |

¹Refer to Table 5E for the components and composition of the excipient, OPADRY ® II Purple 85F100132.
²Removed during processing.

TABLE 5E
Components and Composition
OPADRY ® II Purple 85F100132

| Component | Composition (% w/w) |
|---|---|
| Polyvinyl Alcohol | 46.70 |
| Macrogol/PEG | 23.60 |
| Talc | 17.20 |
| Titanium Dioxide | 10.46 |
| FD&C Blue #2/Indigo | 1.60 |
| Carmine | |
| Carmine | 0.44 |

### EXAMPLES

### Example 1: Manufacturing Process and Process Controls

### Process Description for Tavapadon 0.25 mg Tablets

1. Dispensing: All excipients are dispensed as per the theoretical formula. CVL-751 API was calculated based on Assay value from vendor certificate of analysis (COA). Depending on calculated value of API, amount of Microcrystalline Cellulose was recalculated before dispensing. Also, Magnesium Stearate quantities were dispensed in excess as per formula but recalculated amount needed depending on amount of milled materials.

2. Pre-Mixing: Material microcrystalline cellulose was loaded into 1 cu. ft. bin-blender and blended for 2 min at 12 rpm and then discharged.

3. Co-milling: Pre-mixed material was passed through 197S Comil fitted with 024R screen and then collects milled materials.

4. Pre-Blending: Co-milled material was blended in 1 cu. ft. bin-blender for 2 min at 12 rpm. Transfer API, microcrystalline cellulose, lactose monohydrate, and sodium starch glycolate into the same blender and then mix content for 20 min at 12 rpm. Discharge the blend into 15L Ezi-dock bag at the end of blending process.

5. Co-milling: Pre-blended material was passed through 197S Comil fitted with 024R screen and then collects milled materials in 15L Ezi-dock bag.

6. Pre-lube Blending: Co-milled material and microcrystalline cellulose were transferred to 1 cu. ft. bin-blender using Ezi-dock bag and then contents was blended for 2 min at 12 rpm. Discharge the blend into 20L Ezi-dock bag at the end of blending process.

7. Co-milling: Pre-lube blended material was passed through 197S Comil fitted with 024R screen and then collects milled materials in 20L Ezi-dock bag.

8. Pre-lube Blending 2: Co-milled material was transferred to 1 cu. ft. bin-blender using Ezi-dock bag and then content was blended for 20 min at 12 rpm. Discharge the blend into 20L Ezi-dock bag at the end of blending process.

9. Hand Screening: Magnesium stearate was screened using 20 mesh hand screen and recalculated amount required based on milled materials.

10. Final Blending (Lubrication): Screened Magnesium stearate was added to the same blender and blended in 1 cu. ft. bin-blender for 10 min at 12 rpm and then discharged into 20L Ezi-dock bag. Final blend samples were collected at the end of blending process.

11. Compression: The compression process was performed using a Korsch XL 100 tablet press fitted with ten (10) sets of plain round 6 mm tooling. The compression process was completed at target weights (95-105 mg) and at target thickness (3.25-3.55 mm). During compression process, in-process checks (weights, hardness, thickness, friability, and disintegration time) were performed at beginning, approximately every 30 min (+/−5 min), and at the end of compression process.

TABLE 6
In-Process Controls of the Physical Parameters of the
0.25 mg Tablets and Criteria

| Physical Parameter | Acceptance criteria |
|---|---|
| Appearance | White to off-white tablet |
| Weight Control | Individual 95-105 mg, |
| | average of 10 tablets 97-103 mg |
| Tablet Thickness | 3.35-3.65 mm |
| Hardness | Report result |
| Friability | Not more than 0.5% |
| Disintegration | <10 minutes |

### Process Description for Tavapadon 1.0 mg and 5.0 mg Tablets

1. Dispensing: All excipients are dispensed as per the theoretical formula. CVL-751 API was calculated based on Assay value from vendor certificate of analysis (COA). Depending on calculated value of API, amount of Microcrystalline Cellulose was recalculated before dispensing. Also, Magnesium Stearate quantities were dispensed in excess as per formula but recalculated amount needed depending on amount of milled materials.

2. Pre-Mixing: Microcrystalline cellulose was loaded into 1 or 5 cu. ft. bin-blender and blended for 2 min at 12 rpm.

3. Pre-Blending: Tavapadon, microcrystalline cellulose, lactose monohydrate and sodium starch glycolate into the same 1 or 5 cu. ft. bin-blender and then mix content for 15 min at 12 rpm. Discharge the blend into 20L Ezi-dock bag at the end of blending process.

4. Co-milling: Pre-blended material was passed through 197S Comil fitted with 032R screen and then collects milled materials in 20L Ezi-dock bag.

5. Pre-lube blending: Co-milled material was transferred to 1 or 5 cu. ft. bin-blender using Ezi-dock bag and then content was blended for 10 min at 12 rpm.

6. Hand Screening: Magnesium stearate screened using 20 mesh hand screen.

7. Lube Blending: Transfer screened Magnesium stearate into the same 1 or 5 cu. ft. bin-blender and then content was blended for 5 min at 12 rpm. Discharge the blend into 20L Ezi-dock bag at the end of blending process.

8. Roller Compaction: Roller compaction process was performed at target parameters using knurled rollers, pocket granulator, and 1.0 mm Conidur screen. The granules were collected in 20L Ezi-dock bag. Ribbon sample was collected in the beginning and granule samples were collected at the end of the roller compaction process.

9. Pre-lube blending: Transfer collected granules into 1 cu. ft. bin-blender and mix for 5 min at 12 rpm.

10. Hand Screening: Magnesium stearate was screened using 20 mesh hand screen and recalculate amount required based on collected granules.

11. Final Blending (Lubrication): Screened Magnesium stearate was added to the same blender and blended for 5 min at 12 rpm and then discharged into 20L Ezi-dock bag. Final blend samples were collected at the end of blending process.

12. Compression: The compression process was performed using a Korsch XL 100 tablet press fitted with ten (10) sets of plain round 6 mm tooling. The compression process was completed at target weights (95-105 mg) and at target thickness (3.25-3.55 mm). During compression process, in-process checks (weights, hardness, thickness, friability, and disintegration time) were performed at beginning, approximately every 30 min (+/−5 min), and at the end of compression process.

During compression of the tablets for all dose strengths, in-process checks are performed. Samples of tablets are taken approximately every 30 minutes during the compression of the tablets and are assessed according to the physical parameters provided in Table 7.

TABLE 7
In-Process Controls of the Physical Parameters of the
1.0 and 5.0 mg Tablets and Criteria

| Physical Parameter | Acceptance criteria |
|---|---|
| Appearance | White to off-white tablet |
| Weight Control | Individual 95-105 mg, |
| | average of 10 tablets 97-103 mg |
| Tablet Thickness | 3.25-3.55 mm |
| Hardness | Report result |
| Friability | Not more than 0.8% |
| Disintegration | <10 minutes |

### Process Description for Tavapadon 15 mg Tablets

1. Dispensing: All excipients are dispensed as per the theoretical formula. CVL-751 API was calculated based on Assay value from vendor certificate of analysis (COA). Depending on calculated value of API, amount of microcrystalline cellulose was recalculated before dispensing. Also, magnesium stearate quantities were dispensed in excess as per formula but recalculated amount needed depending on amount of milled materials.

2. Pre-Mixing: Microcrystalline cellulose was loaded into bin-blender and blended.

3. Pre-Blending: Tavapadon drug substance, microcrystalline cellulose, lactose monohydrate and sodium starch glycolate are charged into the same bin-blender and the content is mixed. Discharge the blend into bag at the end of blending process.

4. Co-milling: Pre-blended material was passed through screen and milled materials were collected in bag.

5. Pre-lube blending: Co-milled material was transferred to bin-blender and then content was blended.

6. Hand Screening: Magnesium stearate was screened using hand screen.

7. Lube Blending: Transfer screened Magnesium stearate into the same bin-blender and then content was blended. Discharge the blend into bag at the end of blending process.

8. Roller Compaction: The blend from Step 7 was roller compacted. The granules were collected in bag. Ribbon sample was collected in the beginning and granule samples were collected at the end of the roller compaction process.

9. Pre-lube blending: Transfer collected granules into bin-blender and mix.

10. Hand Screening: Magnesium stearate was screened using hand screen and recalculate amount required based on collected granules.

11. Final Blending (Lubrication): Screened Magnesium stearate was added to the same blender and blended and then discharged into bag. Final blend samples were collected at the end of blending process.

12. Compression: The lubricated blend from Step 11 is compressed into tablets.

13. Coating: The compressed tablets from Step 12 are coated.

During compression of the tablets, in-process checks are performed. Samples of uncoated tablets are assessed according to the physical parameters provided in Table 8.

TABLE 8
In-Process Controls of the Physical Parameters of
the Core Uncoated Tablets and Criteria

| Physical Parameter | Acceptance criteria |
|---|---|
| Appearance | Modified rectangle, white to off-white tablet |
| Weight Control | Individual 285-315 mg, |
| | average of 10 tablets 2.910-3.090 g |
| Tablet Thickness | 3.80-4.30 mm |
| Hardness | 6.0 kp-14.0 kp |
| Friability | Not more than 0.8% |
| Disintegration | <10 minutes |

During coating of the tablets, in-process checks are performed. Samples of coated tablets are assessed according to the physical parameters provided in Table 9.

TABLE 9
In-Process Controls of the Physical Parameters of the
Core Coated Tablets and Criteria

| Physical Parameter | Acceptance criteria |
|---|---|
| Appearance | Modified rectangle, purple to light purple tablet |
| Weight Gain | 3-5% |
| Disintegration | <15 minutes |

### Example 2: Formulation and Manufacturing Process for Tavapadon Tablets 0.25, 1, 5, 10 and 15 Mg

### Phase 2 and Phase 3 Formulations

Following the completion of Phase 1 clinical trials, an immediate-release tablet was selected. In addition to the drug substance, the initial tablet formulations for Phase 2 clinical development were composed of dibasic calcium phosphate (DCP), microcrystalline cellulose, sodium starch glycolate and magnesium stearate.

No drug-excipient interaction studies were formally conducted; however, drug-excipient compatibility was evaluated in tablets via accelerated and long-term stability studies.

These initial development stability studies performed using the DCP formulation showed a trend of increasing impurity content in both room temperature and accelerated stability storage conditions. This trend warranted the replacement of DCP with another diluent (lactose monohydrate) to improve shelf-life. Tablet strengths of 0.25 mg, 1 mg, and 5 mg were produced. Compositions of these Phase 2/3 formulations are described in 10 and Table 10.

TABLE 10
Tavapadon Drug Product Formulations based on Dibasic Calcium Phosphate

| | Strength | | | | | | | | |
|---|---|---|---|---|---|---|---|---|---|
| | 0.25 mg | | 1 mg | | 5 mg | | 15 mg | | |
| | Target Quantity | | | | | | | | |
| | mg Per | | mg Per | | mg Per | | mg Per | | |
| Component | Tablet | % w/w | Tablet | % w/w | Tablet | % w/w | Tablet | % w/w | Function |
| Tavapadon | 0.25 | 0.25 | 1 | 1 | 5 | 5 | 15 | 15 | Active Ingredient |
| Microcrystalline | 63.83 | 63.83 | 63.08 | 63.08 | 59.08 | 59.08 | 177.24 | 59.08 | Diluent |
| Cellulose | | | | | | | | | |
| Di basic Calcium | 31.92 | 31.92 | 31.92 | 31.92 | 31.92 | 31.92 | 95.76 | 31.92 | Diluent |
| Phosphate | | | | | | | | | |
| Sodium Starch | 3.00 | 3.00 | 3.00 | 3.00 | 3.00 | 3.00 | 9.00 | 3.00 | Disintegrant |
| Glycolate | | | | | | | | | |
| Magnesium | 1.00 | 1.00 | 1.00 | 1.00 | 1.00 | 1.00 | 3.00 | 1.00 | Lubricant |
| Stearate | | | | | | | | | |
| Core Tablet | 100.00 | 100.00 | 100.00 | 100.00 | 100.00 | 100.00 | 300.00 | 100.00 | |
| Total | | | | | | | | | |

TABLE 11
Tavapadon Drug Product Formulations (lactose monohydrate based)

| | Strength | | | | | | | | |
|---|---|---|---|---|---|---|---|---|---|
| | 0.25 mg | | 1 mg | | 5 mg | | 15 mg | | |
| | Target Quantity | | | | | | | | |
| | mg Per | | mg Per | | mg Per | | mg Per | | |
| Component | Tablet | % w/w | Tablet | % w/w | Tablet | % w/w | Tablet | % w/w | Function |
| Tavapadon | 0.25 | 0.25 | 1 | 1 | 5 | 5 | 15 | 15 | Active Ingredient |
| Microcrystalline | 63.83 | 63.83 | 63.08 | 63.08 | 59.08 | 59.08 | 177.24 | 59.08 | Diluent |
| Cellulose | | | | | | | | | |
| Lactose | 31.92 | 31.92 | 31.92 | 31.92 | 31.92 | 31.92 | 95.76 | 31.92 | Diluent |
| monohydrate | | | | | | | | | |
| Sodium Starch | 3.00 | 3.00 | 3.00 | 3.00 | 3.00 | 3.00 | 9.00 | 3.00 | Disintegrant |
| Glycolate | | | | | | | | | |
| Magnesium | 1.00 | 1.00 | 1.00 | 1.00 | 1.00 | 1.00 | 3.00 | 1.00 | Lubricant |
| Stearate | | | | | | | | | |
| Core Tablet | 100.00 | 100.00 | 100.00 | 100.00 | 100.00 | 100.00 | 300.00 | 100.00 | |
| Total | | | | | | | | | |

### Phase 2/Phase 3 Drug Product Manufacturing Process

Due to the poor flow properties of the drug substance as indicated by Carr's Index measurement (38-46%) and its potential impact on flow properties of the drug product formulation a dry granulation process was implemented for manufacturing 1 mg and 5 mg strengths. However, low drug loads such as 0.25% were manufactured using a direct compression process successfully.

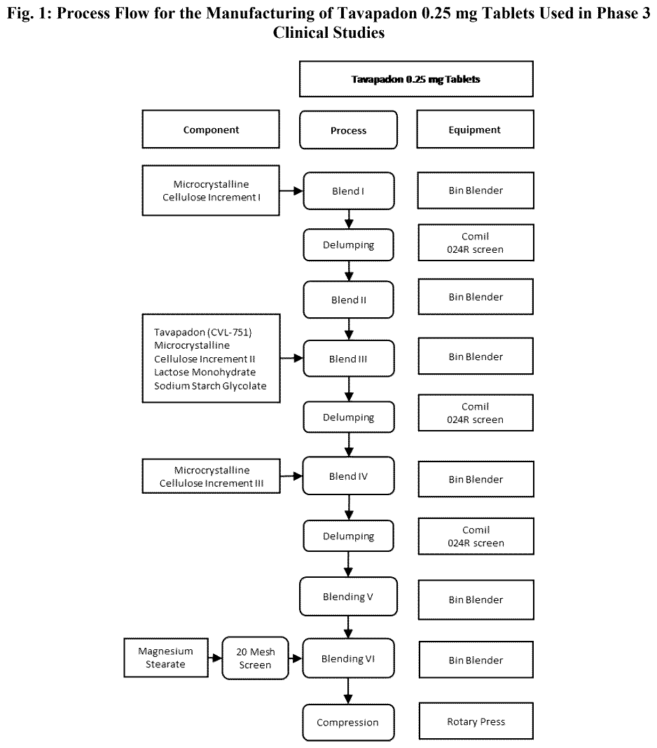

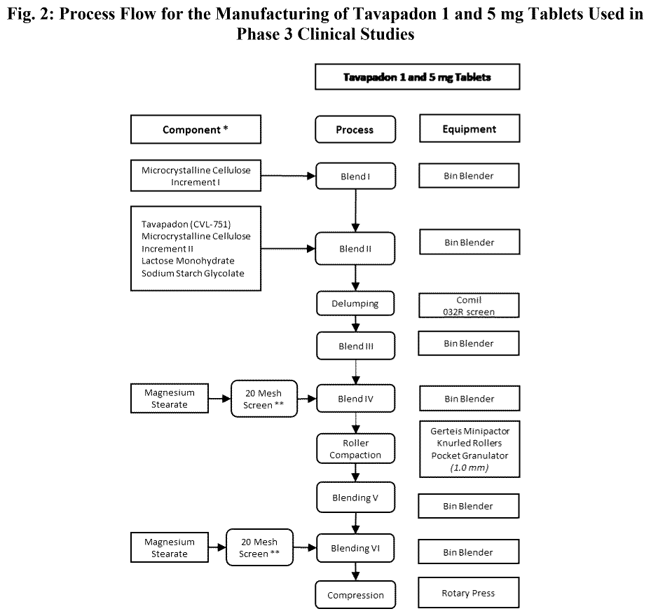

A description of the process flow for the manufacturing of tavapadon 0.25 mg tablets (direct compression process) and tavapadon 1, 5, and 15 mg tablets (roller compaction process) are presented in FIG. 1 and FIG. 2. The manufacturing processes were not changed with the transition from DCP to lactose formulations.

### Commercial Formulations

Following the Phase 3 clinical studies, an Opadry II (Polyvinyl Alcohol based) coating was applied on the tablets cores to arrive at the final commercial formulation which was used to produce the drug product registration stability batches.

Opadry®II, is a high productivity film coating system based on Polyvinyl Alcohol. It offers superior film gloss and short processing times. The product line includes fully formulated dry blend clear, pigmented or white systems for the aqueous film coating of pharmaceutical and nutritional oral pharmaceutical tablets. Opadry II systems are water soluble, pH independent film coatings, which allow immediate disintegration for fast release of drug actives. Two types of pigmented preformulated coating systems were used for the manufacture of drug product commercial image: Opadry II 85F130189 Orange (for tavapadon 0.25 mg and 1 mg tablets) Opadry II 85F100132 Purple (for tavapadon 5 mg, 10 mg and 15 mg tablets)

The film coating was applied at 400 weight gain to provide a homogeneous, aesthetically appealing coverage without impacting drug release profile. The composition of the commercial formulations is described in Table 12.

TABLE 12
Tavapadon Drug Product Registration Stability/Commercial Formulations

| | Strength | | | | | | | | | | |
|---|---|---|---|---|---|---|---|---|---|---|---|
| | 0.25 mg | | 1 mg | | 5 mg | | 10 mg | | 15 mg | | |
| | Target Quantity | | | | | | | | | | |
| | mg Per | | mg Per | | mg Per | | mg Per | | mg Per | | |
| Component | Tablet | % w/w | Tablet | % w/w | Tablet | % w/w | Tablet | % w/w | Tablet | % w/w | Function |
| Tavapadon | 0.25 | 0.25 | 1.00 | 1.00 | 5.00 | 5.00 | 10.00 | 5.00 | 15.00 | 5.00 | Active |
| | | | | | | | | | | | Ingredient |
| Microcrystalline | 63.83 | 63.83 | 63.08 | 63.08 | 59.08 | 59.08 | 118.16 | 59.08 | 177.24 | 59.08 | Diluent |
| Cellulose | | | | | | | | | | | |
| Lactose | 31.92 | 31.92 | 31.92 | 31.92 | 31.92 | 31.92 | 63.84 | 31.92 | 95.76 | 31.92 | Diluent |
| monohydrate | | | | | | | | | | | |
| Sodium Starch | 3.00 | 3.00 | 3.00 | 3.00 | 3.00 | 3.00 | 6.00 | 3.00 | 9.00 | 3.00 | Disintegrant |
| Glycolate | | | | | | | | | | | |
| Magnesium | 1.00 | 1.00 | 1.00 | 1.00 | 1.00 | 1.00 | 2.00 | 1.00 | 3.00 | 1.00 | Lubricant |
| Stearate | | | | | | | | | | | |
| Core Tablet | 100.00 | 100.00 | 100.00 | 100.00 | 100.00 | 100.00 | 200.00 | 100.00 | 300.00 | 100.00 | |
| Total | | | | | | | | | | | |
| Opadry | 4.00 | 4.00 | 4.00 | 4.00 | N/A | N/A | N/A | N/A | N/A | N/A | Film forming |
| OPADRY ® II | | | | | | | | | | | coating layer |
| Orange | | | | | | | | | | | |
| 85F130189 | | | | | | | | | | | |
| OPADRY ® II | N/A | N/A | N/A | N/A | 4.00 | 4.00 | 8.00 | 4.00 | 12.00 | 4.00 | Film forming |
| Purple | | | | | | | | | | | coating layer |
| 85F100132 | | | | | | | | | | | |
| Water ¹ | — | — | — | — | — | — | — | — | — | — | |

| | |
|---|---|
| Total | Opadry II is applied to achieve a 4% coating weight gain to the core tablets |

¹ removed during processing.

### Optimization/Verification of the Commercial Formulation and Process

### Effect of Drug Substance Particle Size

Three different particle size distributions (PSD) of milled drug substance (Small, Medium, Large) in addition to the unmilled drug substance were investigated to evaluate the impact of drug substance PSD on the % release of drug substance from tavapadon 0.25, 1, 5, and 15 mg tablets. Drug substance PSD used for this study is described in Table 13.

TABLE 13
Tavapadon Drug Substance Particle Size Description

| | Particle Size | Specific | |
|---|---|---|---|
| | Distribution | Surface | |
| Tavapadon DS Lot | (D[4,3], μm) | Area m²/g | Designation |
| Lot A | 15 | 3.36 | Small |
| Lot B | 22 | 1.83 | Medium |
| Lot C | 47 | 0.52 | Large |
| Lot D unmilled | 308 | 0.26 | Unmilled |

### Tavapadon 0.25 mg Tablets

Tavapadon 0.25 mg tablets were prepared using a direct compression process using a V-Blender and Korsch XL100 tablet press.

Blending Process: Batches of 400 g of final blend were prepared as follows: Microcrystalline Cellulose (Increment 1) was charged to the 750 mL V-blender and blended for 2 minutes at 25 RPM. Then, the blended cellulose was passed through a 20 mesh hand screen and then returned to the original 750 mL V-blender. The screened material was blended for 2 minutes at 25 RPM. Afterwards, tavapadon drug substance, microcrystalline cellulose (Increment 2), lactose monohydrate, and sodium starch glycolate were all charged to the same V-blender and blended for 20 minutes at 25 RPM. The blended materials were screened through a 20 mesh hand screen and returned to the V-blender. Increment 3 was added to the V-blender, and all components were blended for 2 minutes at 25 RPM and then passed through a 20 mesh hand screen and transferred back into the V-blender and blended for an additional 20 minutes at 25 RPM. Magnesium stearate was manually mixed in the bag with an approximately equal volume of material from the V-blender and screened through a 20-mesh hand screen into the V-blender. The final blend was mixed for 10 minutes at 25 RPM.

Compression: The final blend was then compressed into tablets in the commercial image using Korsch XL100 tablet press under the following conditions:

| Turret speed | 30 | RPM |
|---|---|---|
| Feeder speed | 15 | RPM |
| Cam size | 4-10 | mm |
| Pre-compression force | 0.0 | |
| Main compression force | 4.1 | |
| Avg tablet weight | 0.1005 | g |
| Avg tablet hardness | 5-6 | kp |
| Avg tablet thickness | 3.36 | mm |

### TAVAPADON 1, 5, 15 mg Tablets

Batches of 600 g (1 mg tablets) and 1000 g (5 and 15 mg tablets) of final blends were prepared using roller compaction based process using the following equipment train: blending performed in V-Blender, dry granulation performed using TF-mini roller compactor and tablets were compressed using Korsch XL100 tablet press. All process parameters for each strength were kept the same for all different PSD lots.

Blending Process: Microcrystalline cellulose (Increment 1) was charged into an appropriate size V-shell blender and blended for 2 minutes at 25 RPM. Tavapadon drug substance, microcrystalline cellulose (Increment 2), lactose monohydrate, and sodium starch glycolate were then charged into the bin. The components in the bin were blended for 15 minutes at 25 RPM. The mixture was passed through a 20-mesh hand screen and blended again for 10 minutes at 25 RPM. Then, an approximately equal volume of blended material was removed and manually mixed for 30 seconds with the bag containing the intragranular magnesium stearate. The manually rinsed magnesium stearate was pushed through an 18-mesh hand screen and charged to the V-shell blender containing the remaining mixture and blended for 5 minutes at 25 RPM.

The final blend was roller compacted using TF-mini roller compactor to obtain ribbon samples at about 0.69 solid fraction under the following conditions:

| | |
|---|---|
| Screw speed | 15 kg/cm² |
| Roller speed | 4 kg/cm² |
| Roll pressure | 25 kg/cm² |

The granulated blend was milled using oscillating granulator with an 18 mesh screen and charged into the original V-blender. Then, an approximately equal volume of milled material was removed and manually mixed for 30 seconds with the bag containing the extragranular magnesium stearate. The manually rinsed magnesium stearate was pushed through an 18-mesh hand screen and charged to the V-blender containing the remaining milled material and blended for 5 minutes at 25 RPM.

Compression: The final blend was then compressed into tablets in the commercial image using Korsch XL100 tablet press under the following conditions:

| Compression parameter | 1 mg | 5 mg | 15 mg |
|---|---|---|---|
| Turret speed (RPM) | 50 | 50 | 50 |
| Feeder speed (RPM) | 25 | 25 | 25 |
| Cam size (mm) | 0-6 | 0-6 | 4-10 |
| Pre-compression force (kN) | 0.0 | 0.0 | 0.0 |
| Main compression force (kN) | 5.9 | 4.7 | 17.0 |
| Tablet weight (g) | 0.1000 | 0.1000 | 0.3000 |
| Avg tablet hardness (kp) | 3.5-4.5 | 5.0-6.0 | 11.0-13.0 |
| Avg tablet thickness | 2.80 | 3.70 | 4.10 |

### Results:

No impact of the drug substance particle size on the processing (manufacturability) of the tested dosage strengths was reported. No impact on blend flow was reported, and all strengths met their in-process specifications of tablets weight, hardness, thickness, and friability.

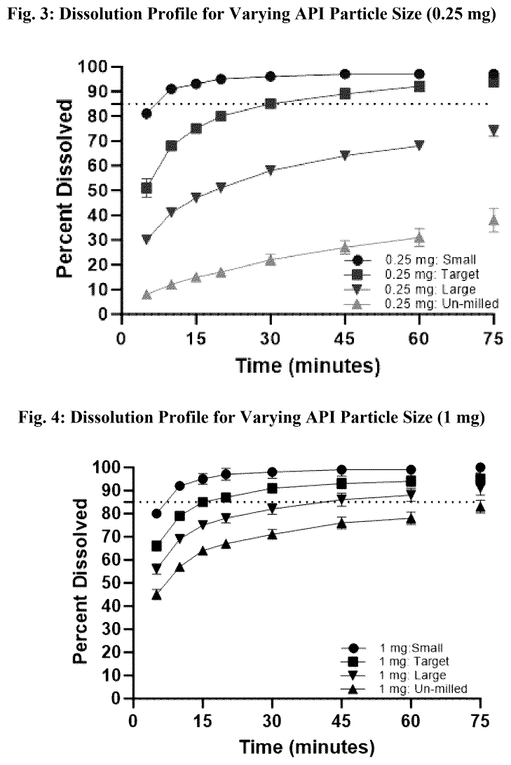

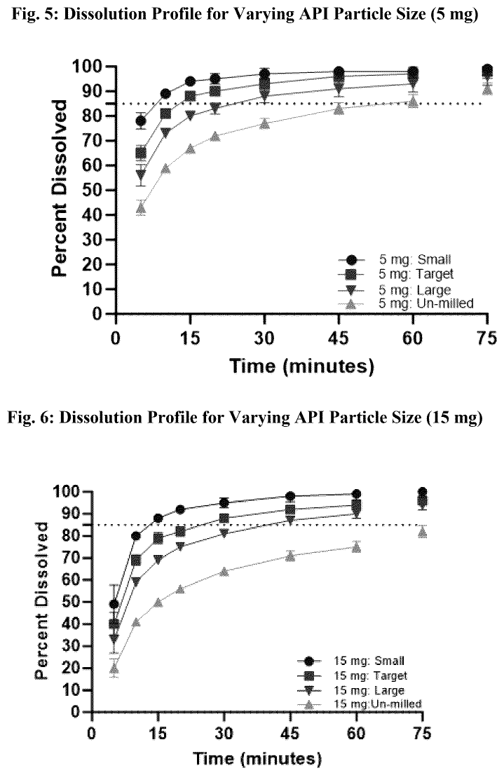

The dissolution results from the varying tavapadon drug substance particle sizes are provided in FIG. 3 to FIG. 6 and summarized in Table 14 to Table 17.

TABLE 14
Dissolution Data for Varying API Particle Size (0.25 mg)

| | Mean % Release | | | | | | | |
|---|---|---|---|---|---|---|---|---|
| Particle Size | Time (minutes) | | | | | | | |
| Description | 5 | 10 | 15 | 20 | 30 | 45 | 60 | 75 |
| Small | 81 | 91 | 93 | 95 | 96 | 97 | 97 | 97 |
| Medium | 51 | 68 | 75 | 80 | 85 | 89 | 92 | 94 |
| Large | 30 | 41 | 47 | 51 | 58 | 64 | 68 | 74 |
| Unmilled | 8 | 12 | 15 | 17 | 22 | 27 | 31 | 38 |

TABLE 15
Dissolution Data for Varying API Particle Size (1 mg)

| | Mean % Release | | | | | | | |
|---|---|---|---|---|---|---|---|---|
| Particle Size | Time (minutes) | | | | | | | |
| Description | 5 | 10 | 15 | 20 | 30 | 45 | 60 | 75 |
| Small | 80 | 92 | 95 | 97 | 98 | 99 | 99 | 100 |
| Medium | 66 | 79 | 85 | 87 | 91 | 93 | 94 | 95 |
| Large | 56 | 69 | 75 | 78 | 82 | 86 | 88 | 91 |
| Unmilled | 45 | 57 | 64 | 67 | 71 | 76 | 78 | 83 |

TABLE 16
Dissolution Data for Varying API Particle Size (5 mg)

| | Mean % Release | | | | | | | |
|---|---|---|---|---|---|---|---|---|
| Particle Size | Time (minutes) | | | | | | | |
| Description | 5 | 10 | 15 | 20 | 30 | 45 | 60 | 75 |
| Small | 78 | 89 | 94 | 95 | 97 | 98 | 98 | 99 |
| Medium | 65 | 81 | 88 | 90 | 93 | 96 | 97 | 98 |
| Large | 56 | 73 | 80 | 83 | 88 | 91 | 93 | 96 |
| Unmilled | 43 | 59 | 67 | 72 | 77 | 83 | 86 | 91 |

TABLE 17
Dissolution Data for Varying API Particle Size (15 mg)

| | Mean % Release | | | | | | | |
|---|---|---|---|---|---|---|---|---|
| Particle Size | Time (minutes) | | | | | | | |
| Description | 5 | 10 | 15 | 20 | 30 | 45 | 60 | 75 |
| Small | 49 | 80 | 88 | 92 | 95 | 98 | 99 | 100 |
| Medium | 40 | 69 | 79 | 82 | 88 | 92 | 94 | 96 |
| Large | 33 | 59 | 69 | 75 | 81 | 87 | 90 | 94 |
| Unmilled | 20 | 41 | 50 | 56 | 64 | 71 | 75 | 82 |

To evaluate dissolution profile comparability F₂ analyses were conducted as per the FDA Guidance for Industry: Dissolution Testing of Immediate Release Solid Oral Dosage Forms, and Dissolution Testing and Acceptance Criteria for Immediate Release Solid Oral Dosage Form Drug Products Containing High Solubility Drug Substances which are incorporated by reference. F₂ comparisons were performed comparing all particle size samples to the medium particle size sample. Profiles with an F₂ value ≥50 are considered similar. F₂ values for the particle sizes evaluated in this study are shown in Table 18-Table 21. The drug substance particle size distribution was determined to be impactful to the performance of the drug product.

The F₂ analysis for tavapadon 0.25 mg tablets shows that there is a significant difference between the drug substance at medium particle size and all other sizes studied. The large (D[4,3]=47 μm) and unmilled (D[4,3]=308 μm) materials failed to meet the proposed specifications for drug release of Q=80% at 45 min indicating that the current PSD specifications (D[4,3]≤25 μm) for the tavapadon drug substance are appropriate to ensure quality drug product performance. See Table 18.

TABLE 18
F₂ Comparison Values, 0.25 mg

| Comparison | F2 Value |
|---|---|
| Medium vs. Small | 36 |
| Medium vs. Large | 30 |
| Medium vs. | 12 |
| Unmilled | |

The F₂ analysis for tavapadon 1 mg tablets shows that there is a significant difference between the drug substance at medium particle size compared to small and unmilled drug substances. The unmilled (D[4,3]=308 μm) material failed to meet the proposed specifications for drug release of Q=80% at 45 min indicating that the current PSD specifications (D[4,3]≤25 μm) for the tavapadon drug substance are appropriate to ensure quality drug product performance. See Table 19.

TABLE 19
F₂ Comparison Values, 1 mg

| Comparison | F2 Value |
|---|---|
| Medium vs. Small | 47 |
| Medium vs. Large | 53 |
| Medium vs. | 36 |
| Unmilled | |

The F₂ analysis for tavapadon 5 mg tablets shows that there is a significant difference between the drug substance at medium particle size compared to unmilled drug substance. The unmilled (D[4,3]=308 μm) material failed to meet the proposed specifications for drug release of Q=80% at 45 min indicating that the current PSD specifications (D[4,3]≤25 μm) for the tavapadon drug substance are appropriate to ensure quality drug product performance. See Table 20.

TABLE 20
F₂ Comparison Values, 5 mg

| Comparison | F2 Value |
|---|---|
| Medium vs. Small | 53 |
| Medium vs. Large | 57 |
| Medium vs. | 39 |
| Unmilled | |

The F₂ analysis for tavapadon 15 mg tablets shows that there is a significant difference between the drug substance at medium particle size compared to unmilled drug substance. The unmilled (D[4,3]=308 μm) material failed to meet the proposed specifications for drug release of Q=80% at 45 min indicating that the current PSD specifications (D[4,3]≤25 μm) for the tavapadon drug substance are appropriate to ensure quality drug product performance. See Table 21.

TABLE 21
F₂ Comparison Values, 15 mg

| Comparison | F₂ Value |
|---|---|
| Medium vs. Small | 53 |
| Medium vs. Large | 56 |
| Medium vs. | 31 |
| Unmilled | |

The effect of two levels of sodium starch glycolate (1% and 5%) were evaluated in tavapadon tablets cores of 0.25 mg, 1 mg and 15 mg tablets. The 1 mg and 15 mg dose strengths were selected as the worst case in terms of dissolution methods challenge from a solubility perspective in their respective dissolution media. Tavapadon 0.25 mg tablets were prepared using a direct compression process using a V-Blender and Korsch XL100 tablet press similar to the process described in impact of drug substance particle size section above. The final blends of 1 mg and 15 mg tablets were prepared using a roller compaction based process using the following equipment train: blending performed in V-Blender dry granulation performed using TF-mini roller compactor and tablets were compressed using Korsch XL100 tablet press similar to the process described in the impact of drug substance particle size section above.

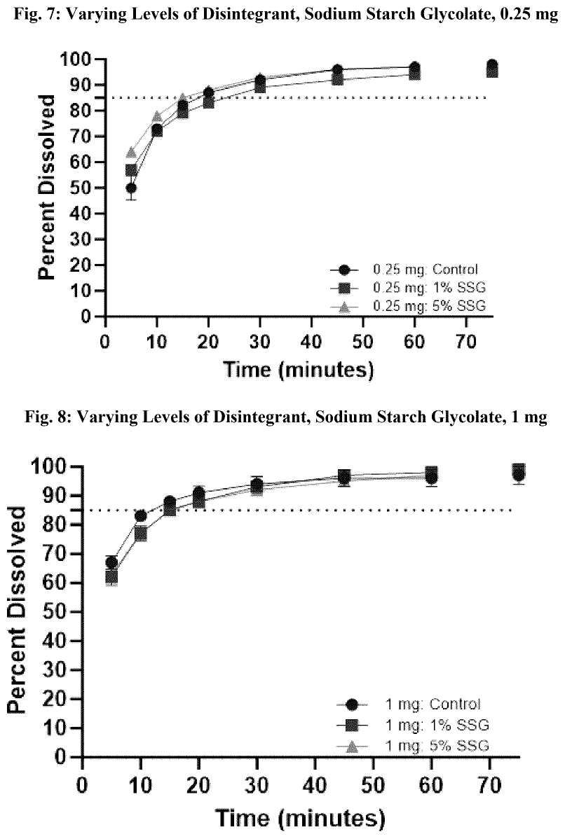

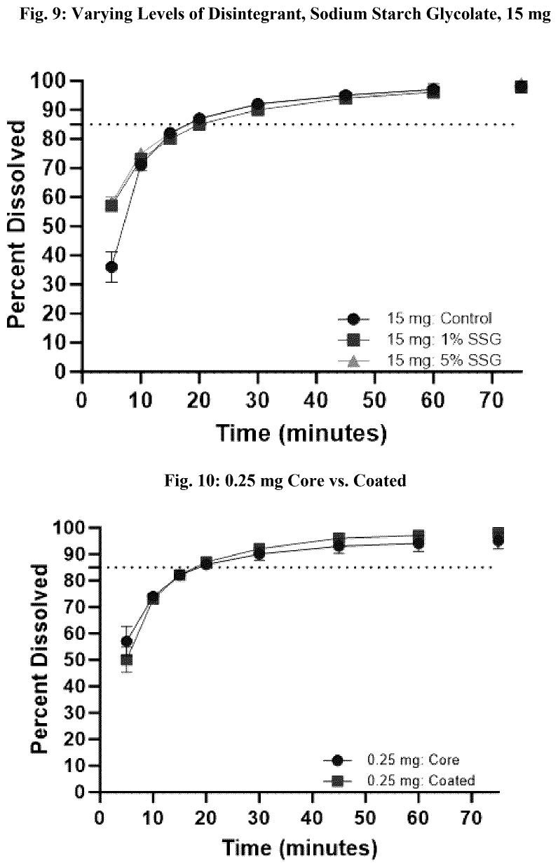

No difference in the release profiles as a function of disintegrant levels between 1% and 5% was observed in the three tablet strengths (FIG. 7-FIG. 9 and Table 22-Table 24).

In addition, the impact of the level of disintegrant was evaluated at different hardness levels of tavapadon 15 mg tablets as worst case for dissolution. Both disintegrant levels (1% and 5%) performed similarly in tablets compressed at target or high hardness levels. The level of disintegrant set at 3% in the final formulation was deemed adequate.

TABLE 22
Summarized Dissolution Profiles for
Sodium Starch Glycolate, 0.25 mg

| | Mean % Release | | | | | | | |
|---|---|---|---|---|---|---|---|---|
| Sample | Time (minutes) | | | | | | | |
| Description | 5 | 10 | 15 | 20 | 30 | 45 | 60 | 75 |
| Target Formulation | 50 | 73 | 82 | 87 | 92 | 96 | 97 | 98 |
| (Control) | | | | | | | | |
| 1% SSG | 57 | 72 | 79 | 83 | 89 | 92 | 94 | 95 |
| 5% SSG | 64 | 78 | 85 | 88 | 93 | 96 | 97 | 98 |

TABLE 23
Summarized Dissolution Profiles
for Sodium Starch Glycolate, 1 mg

| | Mean % Release | | | | | | | |
|---|---|---|---|---|---|---|---|---|
| | Time (minutes) | | | | | | | |
| Sample Description | 5 | 10 | 15 | 20 | 30 | 45 | 60 | 75 |
| Target Formulation | 67 | 83 | 88 | 91 | 94 | 96 | 96 | 97 |
| (Control) | | | | | | | | |
| 1% SSG | 62 | 77 | 85 | 88 | 93 | 97 | 98 | 99 |
| 5% SSG | 63 | 77 | 85 | 88 | 92 | 95 | 97 | 98 |

TABLE 24
Summarized Dissolution Profiles for
Sodium Starch Glycolate, 15 mg

| | Mean % Release | | | | | | | |
|---|---|---|---|---|---|---|---|---|
| | Time (minutes) | | | | | | | |
| Sample Description | 5 | 10 | 15 | 20 | 30 | 45 | 60 | 75 |
| Target Formulation | 36 | 71 | 82 | 87 | 92 | 95 | 97 | 98 |
| (Control) | | | | | | | | |
| 1% SSG | 57 | 73 | 80 | 85 | 90 | 94 | 96 | 98 |
| 5% SSG | 58 | 75 | 82 | 87 | 92 | 95 | 97 | 99 |

### Effect of Magnesium Stearate (Lubricant) Blending Time

The effect of lubricant blending time on tablet dissolution was investigated using DoE studies conducted on tavapadon 0.25 mg, 1, 5, and 15 mg tablets. Lubrication time of final blend for tavapadon 0.25 mg tablets was changed between 7-13 min (10 min target). No practical impact of final lubrication time between 7-13 min was observed on any drug product quality attributes. Final lubrication time of 10 min was deemed adequate.

Lubrication time of pre-roller compaction and final blend for tavapadon 1, 5, and 15 mg tablets was changed between 3-7 min (5 min target). No practical impact of final lubrication time between 3-7 min was observed on any drug product quality attributes. Final lubrication time of 5 min was deemed adequate.

### Effect of Coating and Coating Weight Gain

Tavapadon drug product was developed as an immediate release oral dosage form with a film coat. Tavapadon 0.25 mg, 1 mg, 5 mg, 10 mg and 15 mg tablets (coated and cores) were analyzed to determine the effects of the coating on the dissolution profiles.

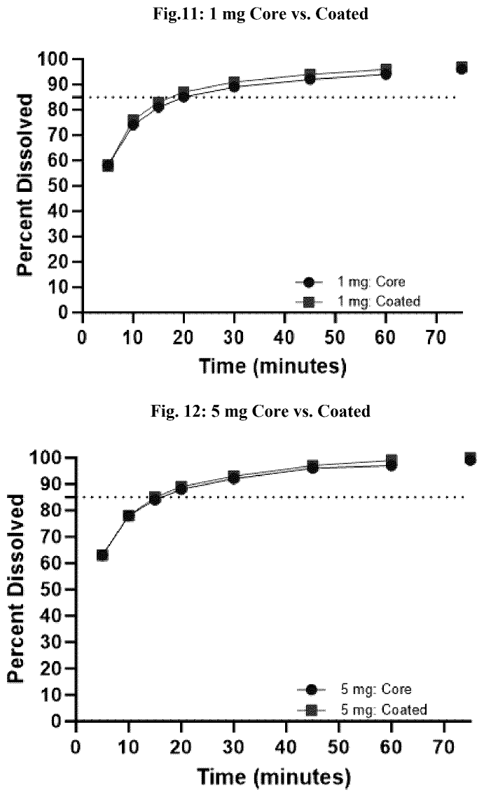

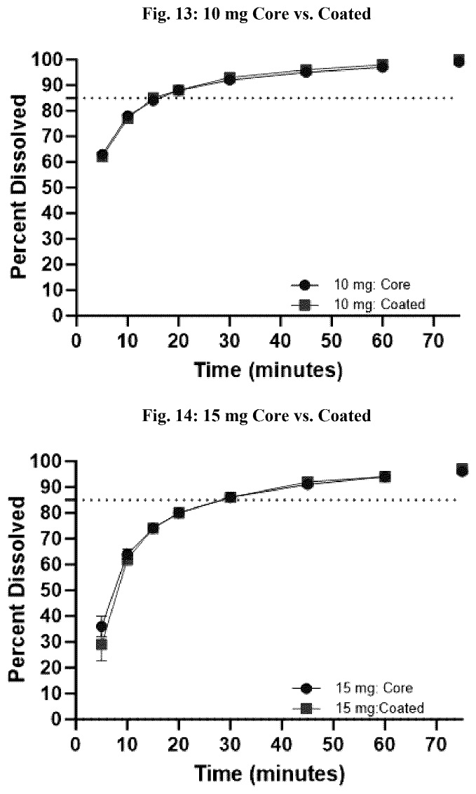

Initial coating studies produced tablets at 3% target weight gain but was found that the coating coverage was not visually uniform, and consequently the target weight gain was increased to 4% instead. The 4% target weight gain produced tablet with more uniform coating coverage. This increase in coating weight gain had no impact on the release profile of tavapadon when compared to the core tablets. The dissolution profiles of coated tablets at 4% weight gain vs. core tablets are similar for all strengths. The coated vs. core results are provided in FIG. 10 to FIG. 14 and summarized in Table 25 to Table 29.

TABLE 25
Summarized Dissolution Profiles 0.25 mg Core vs. Coated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 0.25 mg core | 57 | 74 | 82 | 86 | 90 | 93 | 94 | 95 |
| 0.25 mg coated | 50 | 73 | 82 | 87 | 92 | 96 | 97 | 98 |

TABLE 26
Summarized Dissolution Profiles 1 mg Core vs. Coated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 1 mg core | 58 | 74 | 81 | 85 | 89 | 92 | 94 | 96 |
| 1 mg coated | 58 | 76 | 83 | 87 | 91 | 94 | 96 | 97 |

TABLE 27
Summarized Dissolution Profiles 5 mg Core vs. Coated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 5 mg core | 63 | 78 | 84 | 88 | 92 | 96 | 97 | 99 |
| 5 mg coated | 63 | 78 | 85 | 89 | 93 | 97 | 99 | 100 |

TABLE 28
Summarized Dissolution Profiles 10 mg Core vs. Coated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 10 mg core | 63 | 78 | 84 | 88 | 92 | 95 | 97 | 99 |
| 10 mg coated | 62 | 77 | 85 | 88 | 93 | 96 | 98 | 100 |

TABLE 29
Summarized Dissolution Profiles 15 mg Core vs. Coated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 15 mg core | 36 | 64 | 74 | 80 | 86 | 91 | 94 | 96 |
| 15 mg coated | 29 | 62 | 74 | 80 | 86 | 92 | 94 | 97 |

### Example 3: Stability Study of Tavapadon 0.25, 1, 5, and 15 Mg Tablets

### General

Satisfactory stability evaluation for clinical batches of tavapadon 0.25 mg, 1 mg tablets and 5 mg tablets has been completed through 6 months at 40° C./75% RH as accelerated condition, 60 months at 25° C./60% RH as long term storage conditions and through 3 months at 5° C./60% RH.

Satisfactory stability evaluation for a clinical batch of tavapadon 15 mg tablets has been completed through 6 months at 40° C./75% RH as accelerated conditions, 60 months at 25° C./60% RH as long term storage conditions and through 6 months at 5° C./60% RH.

In addition, a stability evaluation of tavapadon 0.25 mg, 1.0 mag and 5.0 mg tablets in PVC/foil blisters has been initiated and data is available through 24 months for the long term storage condition of 25° C./60% RH and 6 months for the accelerated condition of 40° C./75% RH. After 24 months at long term, the data indicates similar trends to the previous stability observed in the HDPE containers. Testing of tavapadon tablets will continue concurrent with the clinical study per the following stability protocol (Table 30).

TABLE 30
Stability Protocol

| | Storage Period | | | | | | | | | |
|---|---|---|---|---|---|---|---|---|---|---|
| Stability | 1 | 3 | 6 | 9 | 12 | 18 | 24 | 36 | 48 | 60 |
| Condition | mo | mo | mo | mo | mo | mo | mo | mo | mo | mo |
| 40° C./75% RH | X | X | X | — | — | — | — | — | — | — |
| 25° C./60% RH | X | X | X | X | X | X | X | X | X | X |
| 30° C./65% RH | (X) | (X) | (X) | | | | | | | |

A photostability study in accordance with ICH Option 2 conditions has also been conducted.

The stability samples were evaluated using a UPLC method for the active substance and degradation products/impurities. The drug product was also evaluated for appearance, water content, dissolution and disintegration.

The stability of tavapadon tablets in HDPE bottles is supportive of tablets stored in PVC/foil blisters. A stability study in PVC/foil blisters confirms a similar stability and is monitored concurrently with the clinical study.

### Container Closure

Stability samples of drug product have been packaged in high density polyethylene (HDPE) bottles with an induction sealed closure.

A confirmatory stability study has been set up according to the stability protocol listed above on the 0.25 mg, 1 mg, 5 mg and placebo tablets packaged in PVC/foil blisters. The initial shelf life in the PVC/foil blisters will be based on the stability observed for the HDPE packaging configuration. There is little to no change on stability based on the representative drug product in HDPE bottles and the blisters offer better environmental protection.

Photostability samples have been stored in a glass Petri dish and the clinical packaging configuration.

### Analytical Methods

The analytical methods used in the stability program are the are suitable to observe impurities related to the drug product.

### Summary and Conclusions

Available data demonstrate that tavapadon 0.25 mg, 1 mg, 5 mg, and 15 mg tablets are stable at 25° C./60% RH through 60 months. Additional stability studies are ongoing concurrent with clinical studies. The stability data supports the storage of tavapadon tablets at 25° C./60% RH as the long term storage condition.

### Supporting Stability

A statistically designed 4 week Accelerated Stability Assessment Program (ASAP) for a development lot of tavapadon 0.25 mg tablets has been performed. A development lot of tavapadon 0.25 mg tablets was stored in open glass bottles and exposed to various temperature, humidities and durations. The conditions and durations are presented in Table 32.

Testing was performed on 0.25 mg tablets containing 0.25% active drug load as this represents the highest proposed excipient to drug ratio that may be utilized in future manufactures of this lactose formulation, maximizing possible drug-excipient interactions. Consequently, the degradation of the API in the formulation is also maximized. All excipients present in the 0.25 mg tablet are the same as in the 15 mg tablet, but in varying proportions, see Table 31.

TABLE 31
Batch Formula for Tavapadon 0.25 mg Tablet

| | Unit Formula |
|---|---|
| Names of Ingredients | (mg/tablet) |
| Tavapadon | 0.25 |
| Microcrystalline cellulose | 63.83 |
| Lactose monohydrate | 31.92 |
| Sodium Starch Glycolate | 3.00 |
| Magnesium Stearate | 1.00 |
| Total Tablet Weight | 100 mg |

The stability samples were subsequently evaluated for appearance and concentrations of the main degradation product(s), RRT 0.50.

No changes in appearance were observed over the duration of the study. The resultant data for RRT 0.50 were used to predict an initial clinical use period for the formulation. The resultant purity data indicates that unspecified degradant RRT 0.50 will be the use period limiting degradant at 25° C./60% RH therefore the statistical analysis on this degradant was used to predict an initial clinical use period for the formulation. Levels of the unspecified degradant RRT 0.50 ranged from 0.09% to 0.21% for all samples tested. The subsequent model predicted an acceptable clinical use period of at least 12 months when stored at 25° C./60% RH. Table 32 summarizes the accelerated stability data obtained for use period limiting degradation products.

TABLE 32
Stability Protocol and Results for Tavapadon 0.25 mg Tablets

| | | Amount (%) of Use |
|---|---|---|
| | | Period Limiting |
| Condition | Duration | Degradant(s) |
| 5° C./5% RH (Control) | 28 days | 0.10 |
| 50° C./75% RH | 28 days | 0.11 |
| 60° C./40% RH | 28 days | 0.14 |
| 70° C./5% RH | 28 days | 0.10 |
| 50° C./75% RH | 14 days | 0.20 |
| 60° C./40% RH | 14 days | 0.21 |
| 70° C./5% RH | 14 days | 0.13 |
| 70°/75% RH | 14 days | 0.19 |
| 80° C./40% RH | 14 days | 0.14 |
| 70° C./75% RH | 1 day | 0.14 |
| 80° C./40% RH | 2 days | 0.09 |

### Example 4: Dissolution Methods

The two 75-minute dissolution methods consisted of using 900 mL of 0.01N HCl with SDS (sodium dodecyl sulfate) dissolution media (0.15% and 0.2% SDS respectively), at 37±0.5° C. with USP Apparatus 2 (paddles) at 75 rpm, where one was for use with the 0.25 mg and 1 mg tablets and the other was for use with the 5 mg, 10 mg, and 15 mg tablets. Samples are collected at 5, 10, 15, 20, 30, 45, 60, and 75 minutes, with an infinity spin from 60 to 75 minutes at 250 rpm. Filtered solutions are analyzed utilizing HPLC at an absorption wavelength of 274 nm. Detailed method conditions for the 75-minute proposed dissolution methods are provided in Table 33.

TABLE 33
75-minute Dissolution Method

| Apparatus | USP 2 (paddles) | |
|---|---|---|
| Agitation Speed | 75 | rpm |
| Dissolution Medium | 0.01N HCl with 0.15% SDS | |
| | for 0.25 and 1 mg tablets | |
| | 0.01N HCl with 0.2% SDS for | |
| | 5 mg, 10 mg, and 15 mg tablets | |
| Medium Volume | 900 | mL |
| Medium Temperature | 37 ± 0.5° C. | |
| Sampling Volume | 5 | mL |

| | |
|---|---|
| Sampling Times | 5, 10, 15, 20, 30, 45, |
| | 60, and 75 minutes |

The drug product, tavapadon tablets, was developed as an immediate release oral dosage form and was evaluated in multiple strengths during the development program, 0.25 mg, 1 mg, 5 mg, 10 mg, and 15 mg. The 15 mg tablet has been utilized in a bioequivalence study to support the planned 10 mg and 15 mg commercial dosage strengths.

The 0.25 mg tablets are processed by blending and direct compression, and the 1 mg, 5 mg, 10 mg and 15 mg tablets are processed by blending, roller compaction, and compression. The 5 mg, 10 mg, and 15 mg tablets are different strengths developed from a common blend; therefore, the formulation remains consistent with differences only in the tablet weights. The dissolution development and analytical method validation work has considered all dosage strengths.

With respect to the dissolution development studies, a standard dissolution vessel volume and temperature were used (USP<711>, 900 mL or 500 mL at 37° C.) throughout the studies.

Since the dosage form is intended to be an immediate release dosage form, time points covering approximately one hour (5, 10, 15, 20, 30, 45, 60 and 75 minutes, with an infinity spin from 60 to 75 minutes at 250 rpm.) were selected for profile evaluations. Filtered solutions are analyzed utilizing HPLC at an absorption wavelength of 274 nm.

The high and low strengths (0.25 mg and 15 mg) were used as a starting point for dissolution development. The 0.25 mg strength would represent one extreme of dissolution rate (fastest release profile) and the 15 mg strength would represent the other extreme of dissolution rate (slowest release profile).

### Results and Discussion

### Solubility of Drug Substance

### Aqueous Solubility

The pH-solubility profile describes the sensitivity of the drug substance solubility to different pH conditions. Solubility of tavapadon was assessed across the physiologically relevant pH range of 1.2 to 6.8 at 37° C. for 2 hours. Six different dissolution media were selected for evaluation.

The aqueous solubility indicated that there was less than 3× the concentration of a 15 mg tablet in 900 mL of media (0.05 mg/mL) and less than 5× the concentration of a 1 mg tablet in 900 mL of media (0.006 mg/mL). Additional solubility testing was performed using media containing surfactant.

The media and solubility results are listed in Table 34.

TABLE 34
Solubility Results

| | Tavapadon |
|---|---|
| | Solubility |
| Media | (mg/mL) |
| 0.1N HCl, ~pH 1.2 | 0.0051 |
| 0.01N HCl, ~pH 2.1 | 0.0051 |
| 0.001N HCl, pH 3.0 | 0.0049 |
| Sodium Acetate, pH 4.5 | 0.0057 |
| Sodium Phosphate, pH 6.8 | 0.0046 |
| Ammonium Bicarbonate, pH 6.8 | 0.0053 |

### Surfactant Solubility

Surfactant media were prepared by adding the appropriate amount of each surfactant to the base media at levels of ten times the critical micelle concentration (CMC). A representative anionic, cationic, and nonionic surfactant were evaluated. The three surfactants tested were Tween 20 (CMC: ˜0.08%), Cetyltrimethyl ammonium bromide (CTAB; CMC: ˜0.035%) and Sodium dodecyl sulfate (SDS; CMC: ˜0.2%).

CTAB and SDS medium showed concentrations above five times the concentration of a 15 mg tablet in 900 mL media, where SDS showed the highest solubility at ten times the CMC level, therefore, further method development was performed with SDS as the chosen surfactant. All of the base media tested showed similar solubility with SDS, from 2.4 to 3.1 mg/mL.

See Table 35 for surfactant solubility results.

TABLE 35
Surfactant Solubility Results

| | Tavapadon |
|---|---|
| | Solubility |
| Media | (mg/mL) |
| 0.1N HCl with 0.8% Tween 20 | 0.0287 |
| 0.01N HCl with 0.8% Tween 20 | 0.0426 |
| 0.001N HCl, pH 3.0 with 0.8% Tween 20 | 0.0468 |
| Sodium Acetate, pH 4.5 with 0.8% Tween 20 | 0.0452 |
| Sodium Phosphate, pH 6.8 with 0.8% Tween 20 | 0.0451 |
| 0.1N HCl with 0.35% CTAB | 0.2190 |
| 0.01N HCl with 0.35% CTAB | 0.2660 |
| 0.001N HCl, pH 3.0 with 0.35% CTAB | 0.2719 |
| Sodium Acetate, pH 4.5 with 0.35% CTAB | 0.2803 |
| Sodium Phosphate, pH 6.8 with 0.35% CTAB | 0.2974 |
| 0.1N HCl with 2% SDS | 2.364 |
| 0.01N HCl with 2% SDS | 3.025 |
| 0.001N HCl, pH 3.0 with 2% SDS | 3.110 |
| Sodium Acetate, pH 4.5 with 2% SDS | 2.992 |
| Sodium Phosphate, pH 6.8 with 2% SDS | 2.752 |

Additional solubility was performed at the conclusion of the method development to confirm the solubility of the tavapadon drug substance in the chosen media (5× for 1 mg in 900 mL: 0.006 mg/mL, 5× for 15 mg in 900 mL: 0.08 mg/mL).

See Table 36 for solubility results.

TABLE 36
Surfactant Solubility Results

| | Tavapadon |
|---|---|
| | Solubility |
| Media | (mg/mL) |
| 0.01N HCl with 0.15% SDS | 0.0590 |
| 0.01N HCl with 0.2% SDS | 0.1454 |

### Sink Evaluation

Dissolution media screen, sink with surfactant was evaluated by analyzing tavapadon (CVL-751) 0.25 mg tablets in 500 mL of media and 15 mg tablets in 900 mL of media. Samples were tested by analyzing five tablets using apparatus 2 (paddles) at 250 rpm. Samples were removed at the 1-hour time point, centrifuged until free and clear of particulate and diluted by five times.

The goal for dissolution is to have a discriminating method, where full release of the product is achieved by the end of the dissolution run time and to also comply with USP General Chapter <1092> requirements for solubility (sink conditions met in the final chosen medium). Base media of 0.01N HCl was tested during the media screening with SDS to aid in solubility, based on the solubility results. During sink testing of the tablets, it was determined that the base media did not achieve sink requirements, and that use of a surfactant was necessary for strengths above 0.25 mg. As 15 mg is the highest strength for tavapadon, the sink values for the 15 mg were used to estimate the amount of surfactant that would be required to achieve a discriminating method across all strengths. This resulted in an estimated surfactant level of 0.125% to 0.2% SDS, where sink would be achieved for the 15 mg tablet.

The medium used during the screening and the results are summarized in Table 37. Sink was defined as 3× the 15 mg strength in 900 mL of media (0.05 mg/mL).

TABLE 37
Media Screen Results

| | | Meets Sink |
|---|---|---|
| | Concentration | Requirements |
| Media | (mg/mL) | (Y/N) |
| 0.01N HCl | 0.007894 | N |
| 0.01N HCl with 0.1% SDS | 0.01683 | N |
| 0.01N HCl with 0.125% SDS | 0.03191 | N |
| 0.01N HCl with 0.15% SDS | 0.06265 | Y |
| 0.01N HCl with 0.175% SDS | 0.08230 | Y |
| 0.01N HCl with 0.2% SDS | 0.08098 | Y |
| 0.01N HCl with 0.3% SDS | 0.08418 | Y |
| 0.01N HCl with 0.4% SDS | 0.08465 | Y |
| 0.01N HCl with 0.5% SDS | 0.08600 | Y |

### Aqueous Dissolution Evaluation

Aqueous dissolution media were evaluated by analyzing tavapadon (CVL-751) 0.25 mg, and 1 mg tablets. Samples were tested at n=6 in medium as listed in Tables 38 and 39. Tavapadon (CVL-751) tablets were analyzed using USP Apparatus 2 (paddles) in 900 mL of dissolution medium at 75 rpm. This study was conducted without surfactant to determine if surfactant was necessary for dissolution testing, as the solubility test does not account for addition of excipients in the formulation that may aid or hinder solubility. The 0.25 mg and 1 mg tablets were used for this testing as these strengths would be least likely to need surfactant, based on solubility of tavapadon. In the event surfactant was needed for these strengths, surfactant would also be required for higher strengths.

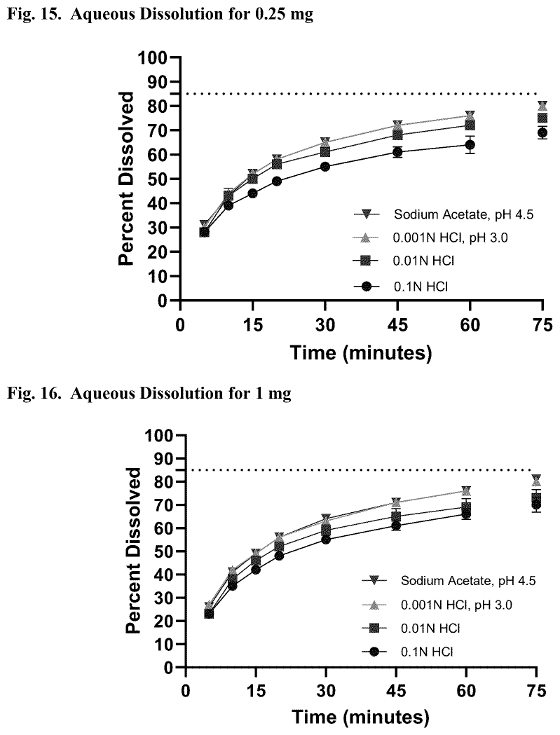

As summarized in Table 38 and Table 39 and FIGS. 15 and 16, full release was not obtained within 75 minutes for all aqueous media and tablet strengths studied, indicating that surfactant would be required.

TABLE 38
Summarized Aqueous Dissolution Profiles for 0.25 mg

| | Mean % Release | | | | | | | |
|---|---|---|---|---|---|---|---|---|
| | Time (minutes) | | | | | | | |
| Medium Description | 5 | 10 | 15 | 20 | 30 | 45 | 60 | 75 |
| 0.1N HCl, ~pH 1.2 | 28 | 39 | 44 | 49 | 55 | 61 | 64 | 69 |
| 0.01N HCl, ~pH 2.1 | 28 | 43 | 50 | 56 | 61 | 68 | 72 | 75 |
| 0.001N HCl, pH 3.0 | 30 | 44 | 52 | 58 | 65 | 72 | 76 | 80 |
| Sodium Acetate, pH 4.5 | 31 | 43 | 52 | 58 | 65 | 72 | 76 | 80 |

TABLE 39
Summarized Aqueous Dissolution Profiles for 1 mg

| | Mean % Release | | | | | | | |
|---|---|---|---|---|---|---|---|---|
| | Time (minutes) | | | | | | | |
| Medium Description | 5 | 10 | 15 | 20 | 30 | 45 | 60 | 75 |
| 0.1N HCl, ~pH 1.2 | 23 | 35 | 42 | 48 | 55 | 61 | 66 | 70 |
| 0.01N HCl, ~pH 2.1 | 23 | 38 | 46 | 52 | 59 | 65 | 69 | 73 |
| 0.001N HCl, pH 3.0 | 27 | 42 | 49 | 56 | 63 | 71 | 76 | 80 |
| Sodium Acetate, pH 4.5 | 26 | 41 | 49 | 56 | 64 | 71 | 76 | 81 |

### Apparatus and Paddle Speed Study

Tavapadon (CVL-751) 1 mg and 15 mg tablets were analyzed at n=6 using USP Apparatus 2 (paddles) at 50 rpm and 75 rpm and Apparatus 1 (baskets) at 100 rpm using 900 mL of 0.01N HCl with 0.15% SDS and 0.01N HCl with 0.2% SDS media (respectively) at 37±0.5° C. Samples were tested to determine the effects of method parameters on dissolution rate.

The percent release in experiments using the chosen method parameters (Apparatus 2, 75 rpm) only increases by 1% for both strengths between the 60- and 75-minute time points. Mounding was observed during the dissolution test in Apparatus 1 at 100 rpm and in Apparatus 2 at 50 rpm, leading to a slower profile that does not achieve full release prior to the infinity spin (250 rpm for 15 minutes). When utilizing paddles at 50 rpm, the percent release between the 60- and 75-minute time points increased by 8% (1 mg) and 14% (15 mg). When utilizing baskets (100 rpm) the percent release between the 60- and 75-minute time points increased by 12% (1 mg) and 25% (15 mg). The 75 rpm paddle speed was determined to be appropriate for testing of all tavapadon strengths.

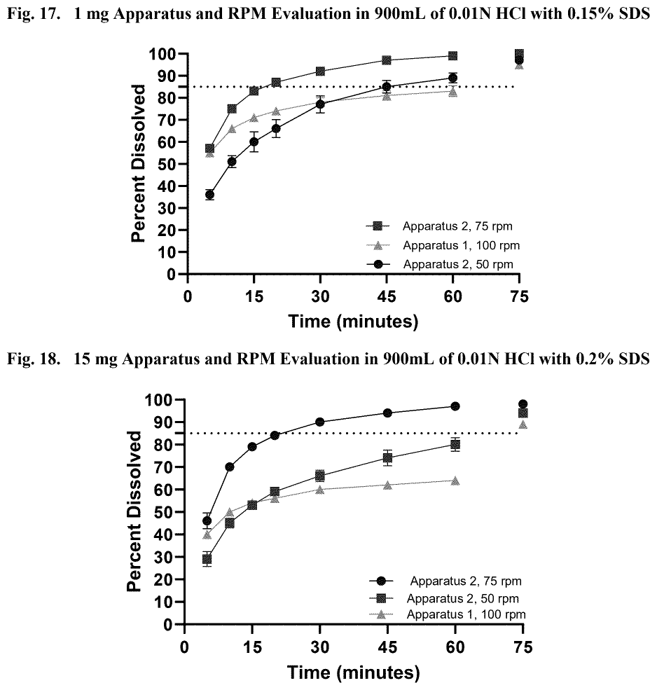

The apparatus and paddle speed comparison results are summarized in Table 40- and Table 41 and FIGS. 17-18.

TABLE 40
Summarized Dissolution Profiles for 1 mg Apparatus and
RPM Evaluation in 900 mL of 0.01N HCl with 0.15% SDS

| | Mean % Release | | | | | | | |
|---|---|---|---|---|---|---|---|---|
| Apparatus and | Time (minutes) | | | | | | | |
| RPM Description | 5 | 10 | 15 | 20 | 30 | 45 | 60 | 75 |
| App 2, 50 rpm | 36 | 51 | 60 | 66 | 77 | 85 | 89 | 97 |
| App 2, 75 rpm | 57 | 75 | 83 | 87 | 92 | 97 | 99 | 100 |
| App 1, 100 rpm | 55 | 66 | 71 | 74 | 78 | 81 | 83 | 95 |

TABLE 41
Summarized Dissolution Profiles for 15 mg Apparatus and
RPM Evaluation in 900 mL of 0.01N HCl with 0.2% SDS

| | Mean % Release | | | | | | | |
|---|---|---|---|---|---|---|---|---|
| Apparatus and | Time (minutes) | | | | | | | |
| RPM Description | 5 | 10 | 15 | 20 | 30 | 45 | 60 | 75 |
| App 2, 50 rpm | 29 | 45 | 53 | 59 | 66 | 74 | 80 | 94 |
| App 2, 75 rpm | 46 | 70 | 79 | 84 | 90 | 94 | 97 | 98 |
| App 1, 100 rpm | 40 | 50 | 54 | 56 | 60 | 62 | 64 | 89 |

Media Screen with Surfactant

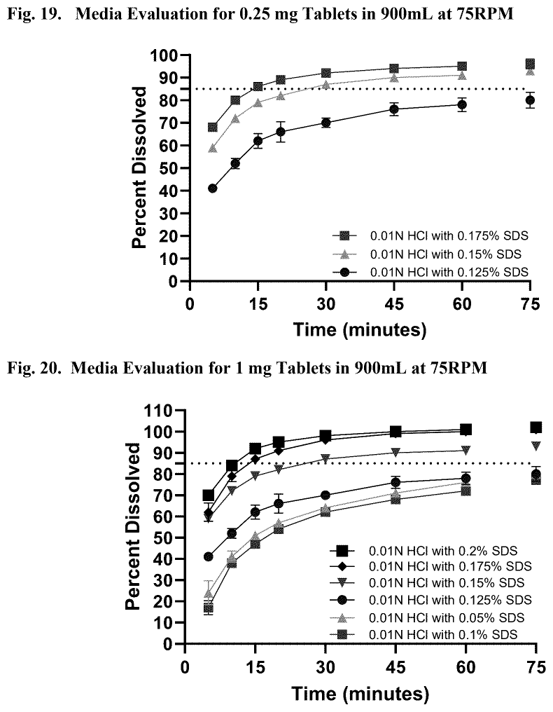

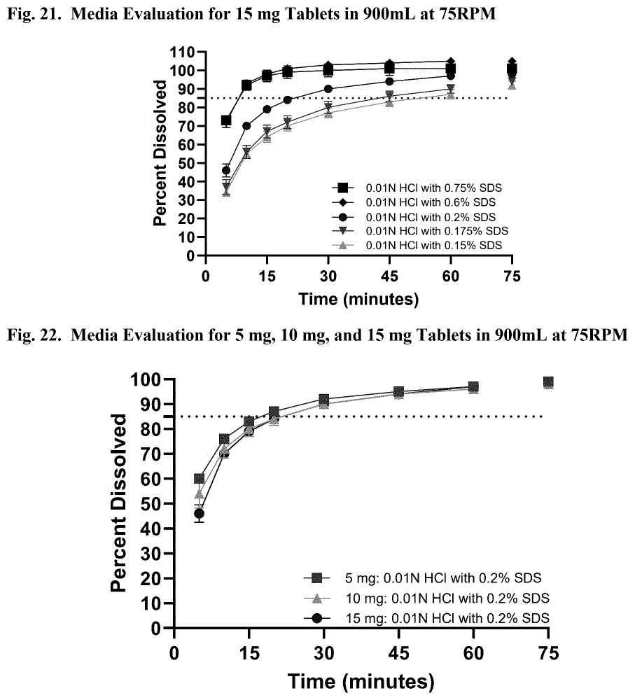

Tavapadon (CVL-751) 0.25 mg, 1 mg and 15 mg tablets were analyzed at n=6 using USP Apparatus 2 (paddles) in 900 mL of dissolution medium at 75 rpm to determine the amount of SDS required for full release. See Tables 42-44 and FIGS. 19-21.

Dissolution testing performed across different levels of SDS in 0.8N HCl with the 0.25 mg and 1 mg tablets determined that any level of SDS below 0.150 SDS would not achieve full release at the infinity time point and percent release was below 90% at the 60-minute time point for the lower levels tested. SDS levels from 0.15% to 0.2% were determined to be appropriate for the dissolution test of both strengths. The 0.15 SDS level in 0.01N HCl media was chosen to be the medium in QM7402 (0.25-1 mg) and 0.2% SDS in 0.01N HCl media was chosen for QM7413 (5-15 mg) as these media were the lowest level of surfactant required to achieve full release at the 60-minute time point, while maintaining a robustness and discriminatory profile.

TABLE 42
Media Evaluation for 0.25 mg Tablets in 900 mL at 75 RPM

| | Mean % Release | | | | | | | |
|---|---|---|---|---|---|---|---|---|
| | Time (minutes) | | | | | | | |
| Medium Description | 5 | 10 | 15 | 20 | 30 | 45 | 60 | 75 |
| 0.01N HCl with 0.125% SDS | 41 | 52 | 62 | 66 | 70 | 76 | 78 | 80 |
| 0.01N HCl with 0.15% SDS | 59 | 72 | 79 | 82 | 87 | 90 | 91 | 93 |
| 0.01N HCl with 0.175% SDS | 68 | 80 | 86 | 89 | 92 | 94 | 95 | 96 |

TABLE 43
Media Evaluation for 1 mg Tablets in 900 mL at 75 RPM

| | Mean % Release | | | | | | | |
|---|---|---|---|---|---|---|---|---|
| | Time (minutes) | | | | | | | |
| Medium Description | 5 | 10 | 15 | 20 | 30 | 45 | 60 | 75 |
| 0.01N HCl with 0.05% SDS | 24 | 41 | 51 | 57 | 64 | 71 | 76 | 80 |
| 0.01N HCl with 0.1% SDS | 17 | 38 | 47 | 54 | 62 | 68 | 72 | 77 |
| 0.01N HCl with 0.125% SDS | 37 | 52 | 63 | 67 | 76 | 81 | 86 | 84 |
| 0.01N HCl with 0.15% SDS | 57 | 75 | 83 | 87 | 92 | 97 | 99 | 100 |
| 0.01N HCl with 0.175% SDS | 62 | 79 | 87 | 91 | 96 | 99 | 100 | 101 |
| 0.01N HCl with 0.2% SDS | 70 | 84 | 92 | 95 | 98 | 100 | 101 | 102 |

TABLE 44
Media Evaluation for 15 mg Tablets in 900 mL at 75 RPM

| | Mean % Release | | | | | | | |
|---|---|---|---|---|---|---|---|---|
| | Time (minutes) | | | | | | | |
| Medium Description | 5 | 10 | 15 | 20 | 30 | 45 | 60 | 75 |
| 0.01N HCl with 0.15% SDS | 34 | 55 | 64 | 70 | 77 | 83 | 87 | 92 |
| 0.01N HCl with 0.175% SDS | 37 | 56 | 67 | 72 | 80 | 86 | 90 | 94 |
| 0.01N HCl with 0.2% SDS | 46 | 70 | 79 | 84 | 90 | 94 | 97 | 98 |
| 0.01N HCl with 0.6% SDS | 72 | 93 | 98 | 101 | 103 | 104 | 105 | 105 |
| 0.01N HCl with 0.75% SDS | 73 | 92 | 97 | 99 | 100 | 101 | 101 | 101 |

Upon concluding the chosen medium for the 15 mg strength (0.01N HCl with 0.2% SDS) the 5 mg and 10 mg strengths were tested to confirm the selected medium. See Table 45 and FIG. 22.

TABLE 45
Media Evaluation for 5 mg, 10 mg, and
15 mg Tablets in 900 mL at 75 RPM

| | Mean % Release | | | | | | | |
|---|---|---|---|---|---|---|---|---|
| | Time (minutes) | | | | | | | |
| Medium Description | 5 | 10 | 15 | 20 | 30 | 45 | 60 | 75 |
| 5 mg: | 60 | 76 | 83 | 87 | 92 | 95 | 97 | 99 |
| 0.01N HCl with 0.2% SDS | | | | | | | | |
| 10 mg: | 54 | 72 | 80 | 84 | 90 | 94 | 96 | 98 |
| 0.01N HCl with 0.2% SDS | | | | | | | | |
| 15 mg: | 46 | 70 | 79 | 84 | 90 | 94 | 97 | 98 |
| 0.01N HCl with 0.2% SDS | | | | | | | | |

### Biorelevant Dissolution Evaluation

Tavapadon (CVL-751) 0.25 mg, 1 mg and 15 mg tablets were tested using fasted simulated gastric fluid (FaSSGF), fasted simulated intestinal fluid (FaSSIF) and fed simulated intestinal fluid (FeSSIF) to evaluate the in vivo dissolution of the drug product. 0.25 mg, 1 mg, and 15 mg were analyzed at n=12.

Tavapadon (CVL-751) tablets do not release completely in any biorelevant media studied. The solubility of Tavapadon (CVL-751) shown in Table 46 is below the sink conditions, sink is defined as 3X the 15 mg strength in 900 mL of media (0.05 mg/mL).

TABLE 46
Tavapadon Solubility in Biorelevant Media

| | Tavapadon |
|---|---|
| | Solubility |
| Media | (mg/mL) |
| FaSSGF | 0.0085 |
| FaSSIF | 0.0060 |
| FeSSIF | 0.0077 |

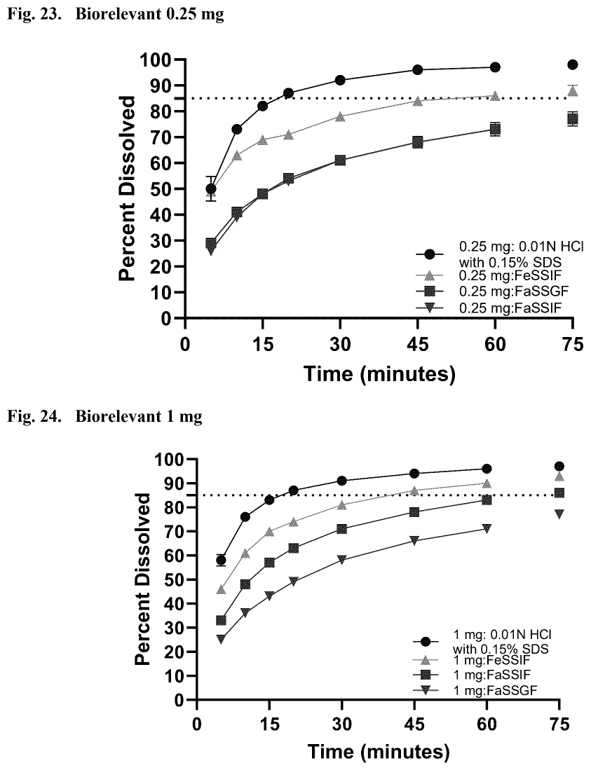

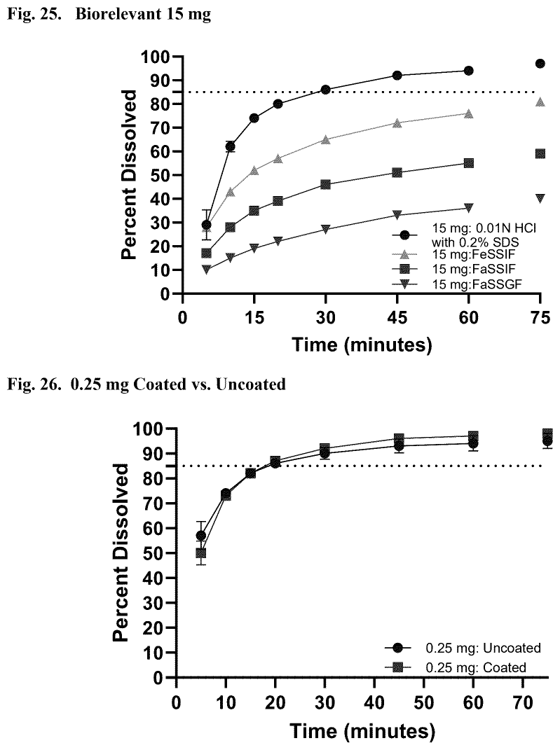

The biorelevant results are summarized in Tables 47-49 and FIGS. 23-25.

TABLE 47
Summarized Dissolution Profiles for 0.25 mg in Biorelevant Media

| | Mean % Release | | | | | | | |
|---|---|---|---|---|---|---|---|---|
| | Time (minutes) | | | | | | | |
| Dissolution Media | 5 | 10 | 15 | 20 | 30 | 45 | 60 | 75 |
| 0.01N HCl with 0.15% SDS | 50 | 73 | 82 | 87 | 92 | 96 | 97 | 98 |
| FaSSGF | 29 | 41 | 48 | 54 | 61 | 68 | 73 | 77 |
| FaSSIF | 26 | 39 | 48 | 53 | 61 | 68 | 73 | 77 |
| FeSSIF | 49 | 63 | 69 | 71 | 78 | 84 | 86 | 88 |

TABLE 48
Summarized Dissolution Profiles for 1 mg in Biorelevant Media

| | Mean % Release | | | | | | | |
|---|---|---|---|---|---|---|---|---|
| | Time (minutes) | | | | | | | |
| Dissolution Media | 5 | 10 | 15 | 20 | 30 | 45 | 60 | 75 |
| 0.01N HCl with 0.15% SDS | 58 | 76 | 83 | 87 | 91 | 94 | 96 | 97 |
| FaSSGF | 25 | 36 | 43 | 49 | 58 | 66 | 71 | 77 |
| FaSSIF | 33 | 48 | 57 | 63 | 71 | 78 | 83 | 86 |
| FeSSIF | 46 | 61 | 70 | 74 | 81 | 87 | 90 | 93 |

TABLE 49
Summarized Dissolution Profiles for 15 mg in Biorelevant Media

| | Mean % Release | | | | | | | |
|---|---|---|---|---|---|---|---|---|
| | Time (minutes) | | | | | | | |
| Dissolution Media | 5 | 10 | 15 | 20 | 30 | 45 | 60 | 75 |
| 0.01N HCl with 0.2% SDS | 29 | 62 | 74 | 80 | 86 | 92 | 94 | 97 |
| FaSSGF | 10 | 15 | 19 | 22 | 27 | 33 | 36 | 40 |
| FaSSIF | 17 | 28 | 35 | 39 | 46 | 51 | 55 | 59 |
| FeSSIF | 28 | 43 | 52 | 57 | 65 | 72 | 76 | 81 |

Coated vs. Uncoated Tablets

Tavapadon (CVL-751) drug product was developed as an immediate release oral dosage form with a film coat. 0.25 mg, 1 mg, 5 mg, 10 mg and 15 mg tablets (coated and uncoated) were analyzed at n=3 to determine the effects of the coating on the dissolution profiles using 0.01N HCl with 0.15% SDS for the 0.25 mg and 1 mg tablets and 0.01N HCl with 0.2% SDS for the 5 mg, 10 mg and 15 mg tablets.

The dissolution profiles of coated vs. uncoated tablets are similar for all strengths. The film coating does not affect the performance of the method. See Table 50 for a summary of the F₂ calculations.

TABLE 50
Coated vs. Uncoated F₂ Calculations
F₂ Calculations Summary

| | |
|---|---|
| 0.25 mg Uncoated to Coated | 73 |
| 1 mg Uncoated to Coated | 87 |
| 5 mg Uncoated to Coated | 94 |
| 10 mg Uncoated to Coated | 94 |
| 15 mg Uncoated to Coated | 93 |

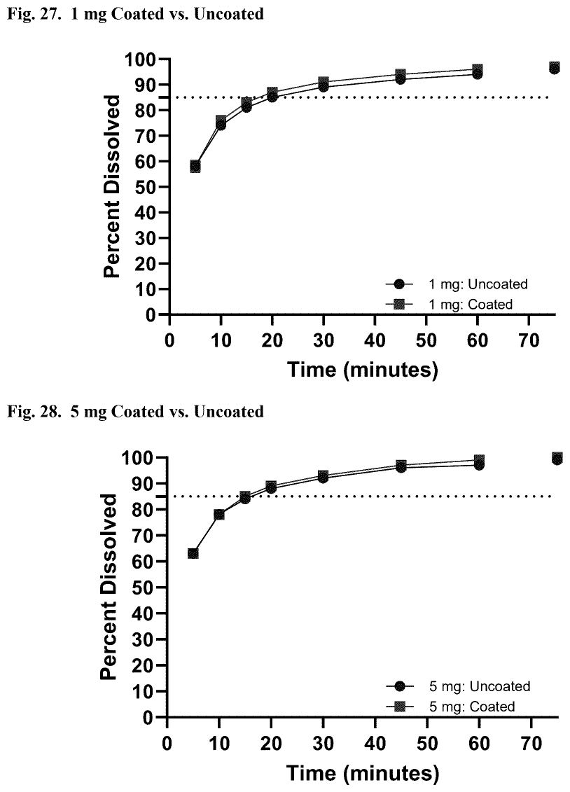

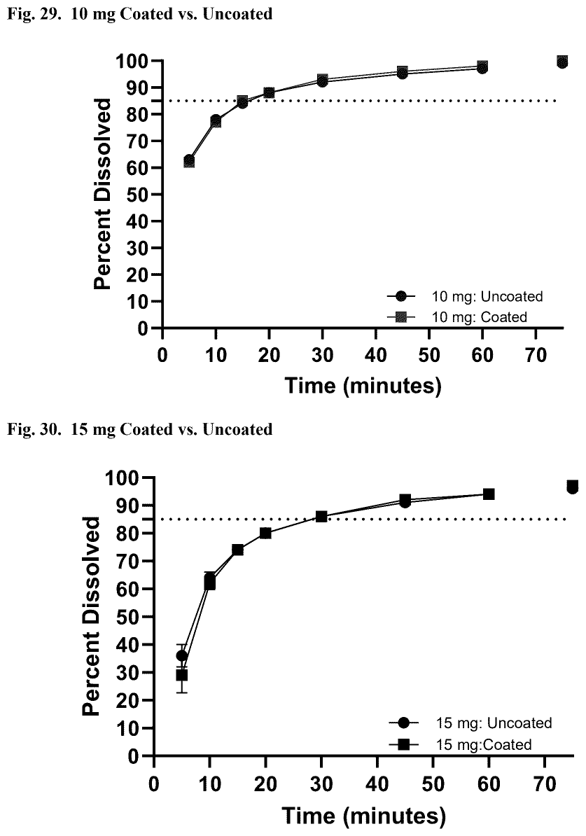

The coated vs. uncoated results are summarized in Tables 51-55 and FIGS. 26-30.

TABLE 51
Summarized Dissolution Profiles 0.25 mg Coated vs. Uncoated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 0.25 mg uncoated | 57 | 74 | 82 | 86 | 90 | 93 | 94 | 95 |
| 0.25 mg coated | 50 | 73 | 82 | 87 | 92 | 96 | 97 | 98 |

TABLE 52
Summarized Dissolution Profiles 1 mg Coated vs. Uncoated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 1 mg uncoated | 58 | 74 | 81 | 85 | 89 | 92 | 94 | 96 |
| 1 mg coated | 58 | 76 | 83 | 87 | 91 | 94 | 96 | 97 |

TABLE 53
Summarized Dissolution Profiles 5 mg Coated vs. Uncoated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 5 mg uncoated | 63 | 78 | 84 | 88 | 92 | 96 | 97 | 99 |
| 5 mg coated | 63 | 78 | 85 | 89 | 93 | 97 | 99 | 100 |

TABLE 54
Summarized Dissolution Profiles 10 mg Coated vs. Uncoated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 10 mg uncoated | 63 | 78 | 84 | 88 | 92 | 95 | 97 | 99 |
| 10 mg coated | 62 | 77 | 85 | 88 | 93 | 96 | 98 | 100 |

TABLE 55
Summarized Dissolution Profiles 15 mg Coated vs. Uncoated

| | Mean % Release | | | | | | | |
|---|---|---|---|---|---|---|---|---|
| | Time (minutes) | | | | | | | |
| Lot Description | 5 | 10 | 15 | 20 | 30 | 45 | 60 | 75 |
| 15 mg uncoated | 36 | 64 | 74 | 80 | 86 | 91 | 94 | 96 |
| 15 mg coated | 29 | 62 | 74 | 80 | 86 | 92 | 94 | 97 |

### Tavapadon Drug Substance Particle Size

Tavapadon (CVL-751) 0.25 mg, 1 mg, 5 mg and 15 mg tablets were manufactured using different lots of drug substance (DS), representing different particle size distributions. This includes un-milled DS, small particle size, large particle size, medium particle size. See Table 56 below for corresponding lot and particle size information.

TABLE 56
Tavapadon Particle Size per Lot

| Tavapadon DS Lot | Average D[4, 3] | Designation |
|---|---|---|
| Lot A | 3.7 μm | Small |
| Lot B | 12 μm | Medium |
| Lot C | 38 μm | Large |
| Lot D un-milled | 56 μm | Un-milled |

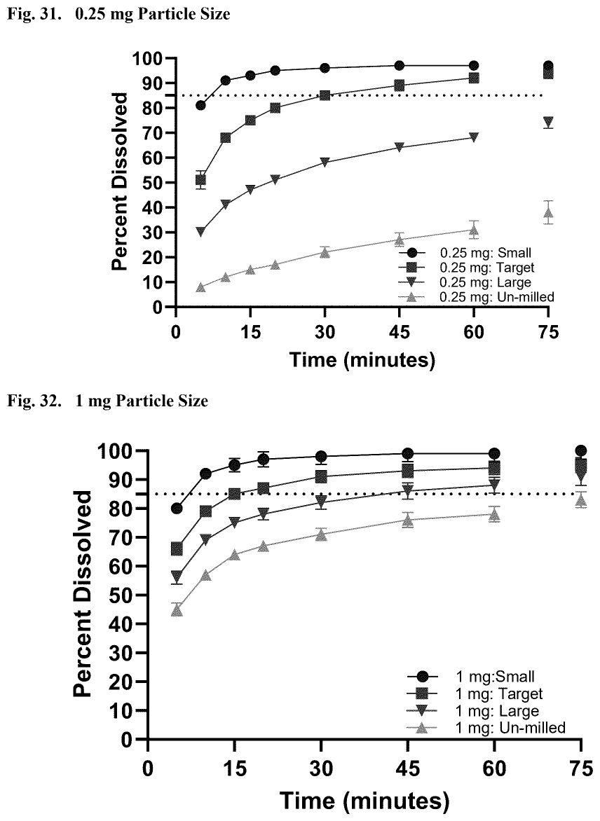

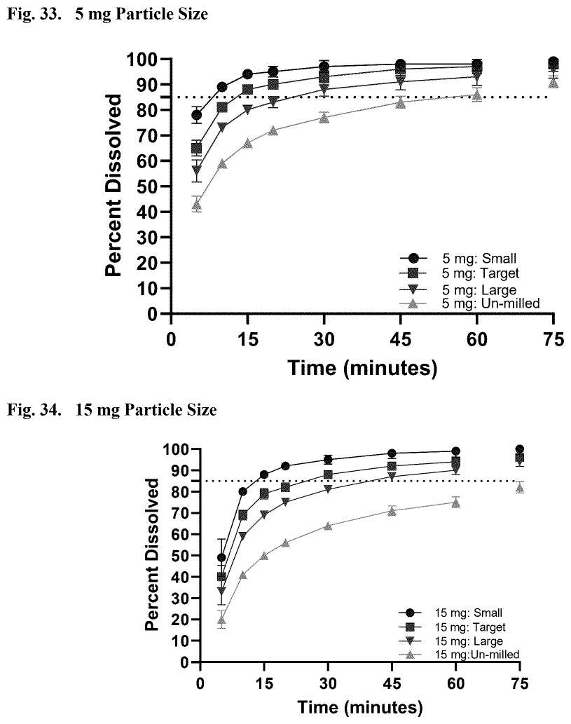

The dissolution results from the varying tavapadon drug substance particle sizes are summarized in Tables 57-64 and FIGS. 31-34.

F₂ comparisons were performed comparing all particle size samples to the medium particle size sample. Profiles are considered similar with an F₂ value of ≥50.

The dissolution conditions were found to be discriminatory for all particle sizes ranges studied.

TABLE 57
Summarized Dissolution Profiles for 0.25 mg Particle Size

| | Mean % Release | | | | | | | |
|---|---|---|---|---|---|---|---|---|
| Particle Size | Time (minutes) | | | | | | | |
| Description | 5 | 10 | 15 | 20 | 30 | 45 | 60 | 75 |
| Small | 81 | 91 | 93 | 95 | 96 | 97 | 97 | 97 |
| Large | 30 | 41 | 47 | 51 | 58 | 64 | 68 | 74 |
| Medium | 51 | 68 | 75 | 80 | 85 | 89 | 92 | 94 |
| Un-milled | 8 | 12 | 15 | 17 | 22 | 27 | 31 | 38 |

TABLE 58

| F₂ Comparison Values, 0.25 mg | |
|---|---|
| Comparison | F₂ Value |
| Medium vs. Small | 36 |
| Medium vs. Large | 30 |
| Medium vs. Un-milled | 12 |

TABLE 59
Summarized Dissolution Profiles for 1 mg Particle Size

| | Mean % Release | | | | | | | |
|---|---|---|---|---|---|---|---|---|
| Particle Size | Time (minutes) | | | | | | | |
| Description | 5 | 10 | 15 | 20 | 30 | 45 | 60 | 75 |
| Small | 80 | 92 | 95 | 97 | 98 | 99 | 99 | 100 |
| Large | 56 | 69 | 75 | 78 | 82 | 86 | 88 | 91 |
| Medium | 66 | 79 | 85 | 87 | 91 | 93 | 94 | 95 |
| Un-milled | 45 | 57 | 64 | 67 | 71 | 76 | 78 | 83 |

TABLE 60

| F₂ Comparison Values, 1 mg | |
|---|---|
| Comparison | F₂ Value |
| Medium vs. Small | 47 |
| Medium vs. Large | 53 |
| Medium vs. Un-milled | 36 |

TABLE 61
Summarized Dissolution Profiles for 5 mg Particle Size

| | Mean % Release | | | | | | | |
|---|---|---|---|---|---|---|---|---|
| Particle Size | Time (minutes) | | | | | | | |
| Description | 5 | 10 | 15 | 20 | 30 | 45 | 60 | 75 |
| Small | 78 | 89 | 94 | 95 | 97 | 98 | 98 | 99 |
| Large | 56 | 73 | 80 | 83 | 88 | 91 | 93 | 96 |
| Medium | 65 | 81 | 88 | 90 | 93 | 96 | 97 | 98 |
| Un-milled | 43 | 59 | 67 | 72 | 77 | 83 | 86 | 91 |

TABLE 62

| F₂ Comparison Values, 5 mg | |
|---|---|
| Comparison | F₂ Value |
| Medium vs. Small | 53 |
| Medium vs. Large | 57 |
| Medium vs. Un-milled | 39 |

TABLE 64

| F₂ Comparison Values, 15 mg | |
|---|---|
| Comparison | F₂ Value |
| Medium vs. Small | 53 |
| Medium vs. Large | 56 |
| Medium vs. Un-milled | 31 |

### Sodium Starch Glycolate (Disintegrant)

Tablets were manufactured with contrived formulations to test the discriminatory power of the method. Tavapadon (CVL-751) 0.25 mg, 1 mg and 15 mg tablets were manufactured with either 1% or 5% sodium starch glycolate (SSG), where microcrystalline cellulose (MCC) was varied to accommodate this change from the typical formulation of 3% SSG. 0.25 mg, 1 mg and 15 mg tablets were tested at n=12.

Dissolution profiles for the 0.25 mg, 1 mg and 15 mg tablets with variable amounts of SSG was not discriminatory within the range studied.

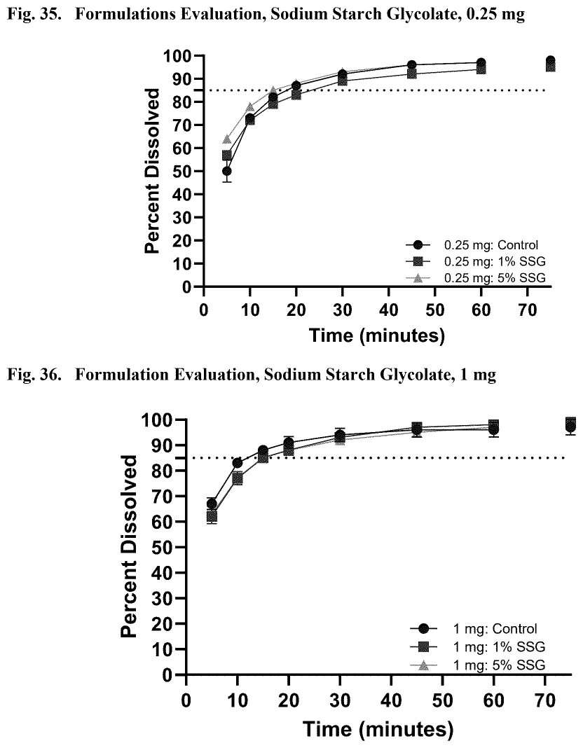

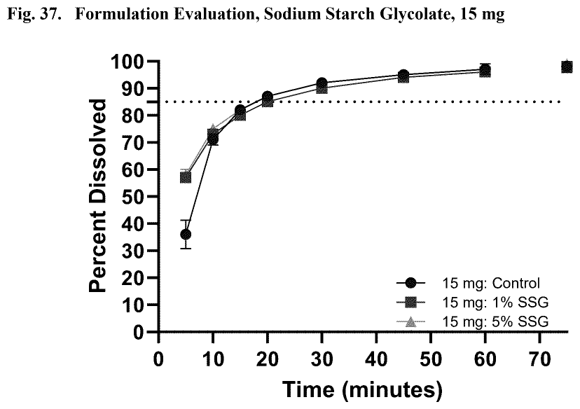

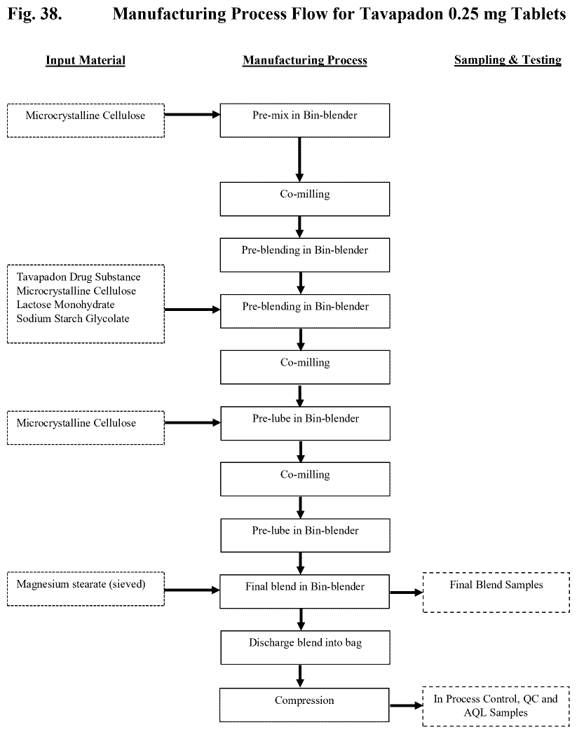

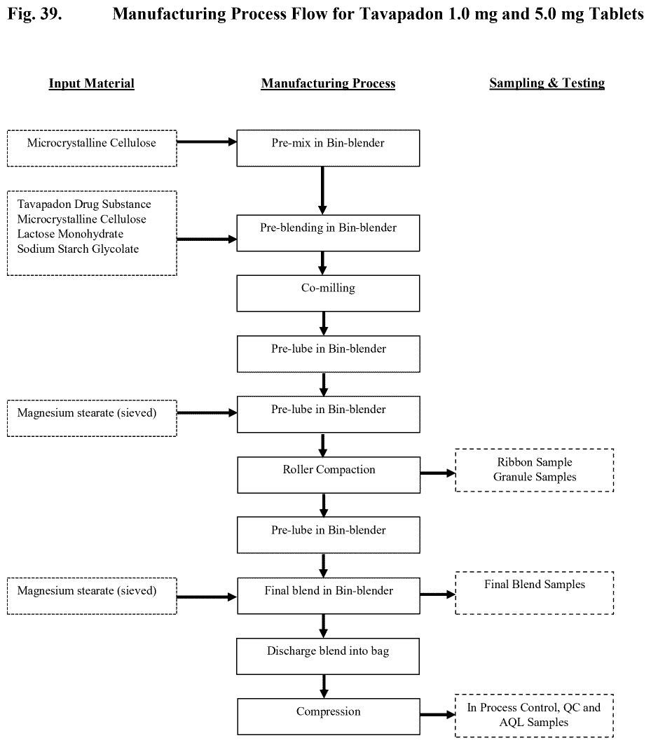

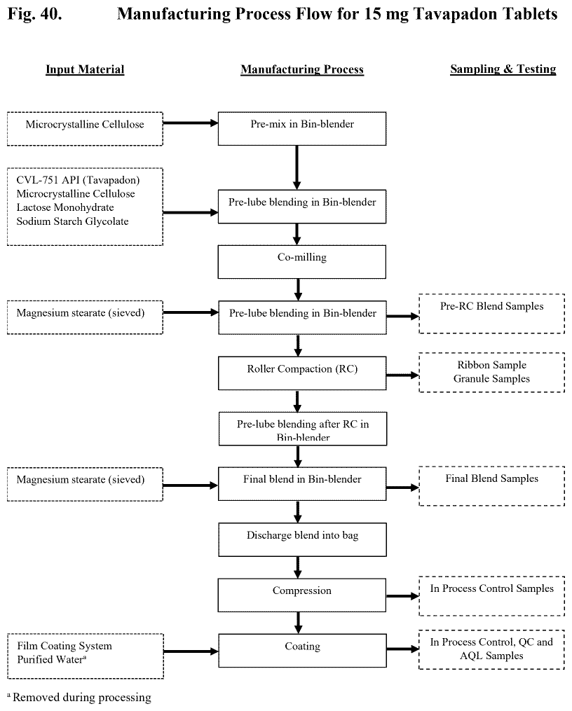

The SSG results are summarized in Tables 65-67 and FIGS. 35-37.

TABLE 65
Summarized Dissolution Profiles for
Sodium Starch Glycolate, 0.25 mg

| | Mean % Release | | | | | | | |
|---|---|---|---|---|---|---|---|---|
| | Time (minutes) | | | | | | | |
| Sample Description | 5 | 10 | 15 | 20 | 30 | 45 | 60 | 75 |
| Target Formulation (Control) | 50 | 73 | 82 | 87 | 92 | 96 | 97 | 98 |
| 1% SSG | 57 | 72 | 79 | 83 | 89 | 92 | 94 | 95 |
| 5% SSG | 64 | 78 | 85 | 88 | 93 | 96 | 97 | 98 |

TABLE 66
Summarized Dissolution Profiles
for Sodium Starch Glycolate, 1 mg

| | Mean % Release | | | | | | | |
|---|---|---|---|---|---|---|---|---|
| | Time (minutes) | | | | | | | |
| Sample Description | 5 | 10 | 15 | 20 | 30 | 45 | 60 | 75 |
| Target Formulation (Control) | 67 | 83 | 88 | 91 | 94 | 96 | 96 | 97 |
| 1% SSG | 62 | 77 | 85 | 88 | 93 | 97 | 98 | 99 |
| 5% SSG | 63 | 77 | 85 | 88 | 92 | 95 | 97 | 98 |

TABLE 67
Summarized Dissolution Profiles for
Sodium Starch Glycolate, 15 mg

| | Mean % Release | | | | | | | |
|---|---|---|---|---|---|---|---|---|
| | Time (minutes) | | | | | | | |
| Sample Description | 5 | 10 | 15 | 20 | 30 | 45 | 60 | 75 |
| Target Formulation (Control) | 36 | 71 | 82 | 87 | 92 | 95 | 97 | 98 |
| 1% SSG | 57 | 73 | 80 | 85 | 90 | 94 | 96 | 98 |
| 5% SSG | 58 | 75 | 82 | 87 | 92 | 95 | 97 | 99 |

### Dissolution Profiles for Tavapadon

TABLE 68
Tavapadon (CVL-751) 0.25 mg Tablets
Lot #1 (Coated)
Lot #2 (Uncoated)

| | Lot #1 | Lot #2 | |
|---|---|---|---|
| | % release | % release | Square of the |
| Time point (min) | (n = 12 mean) | (n = 12 mean) | Difference |
| 5 | 50.391 | 57.373 | 48.7 |
| 10 | 72.973 | 73.688 | 0.5 |
| 15 | 81.700 | 81.989 | 0.1 |
| 20 | 86.849 | 85.761 | 1.2 |
| 30 | 91.880 | 90.168 | 2.9 |
| n (time points)* | | Sum = | 53.5 |
| 5 | | F2 = | 73 |

If F2 ≥ 50, similarly demonstrated profiles

*Number of samplings used where % RSD <20% and to include one time point past 85% mean release. % RSD is relative standard deviation.

TABLE 69
Tavapadon (CVL-751) 1 mg Tablets
Lot #1: (Coated)
Lot #2: (Uncoated)

| | Lot #1 | Lot #2 | |
|---|---|---|---|
| | % release | % release | Square of the |
| Time point (min) | (n = 12 mean) | (n = 12 mean) | Difference |
| 5 | 57.685 | 58.452 | 0.6 |
| 10 | 75.522 | 73.844 | 2.8 |
| 15 | 82.761 | 81.271 | 2.2 |
| 20 | 86.679 | 84.934 | 3.0 |
| 30 | 90.919 | 89.244 | 2.8 |
| n (time points)* | | Sum = | 11.5 |
| 5 | | F2 = | 87 |

If F2 ≥ 50, similarly demonstrated profiles

*Number of samplings used where % RSD <20% and to include one time point past 85% mean release

TABLE 70
Tavapadon (CVL-751) 5 mg Tablets
Lot #1: (Coated)
Lot #2: (Uncoated)

| | Lot #1 | Lot #2 | |
|---|---|---|---|
| | % release | % release | Square of the |
| Time point (min) | (n = 12 mean) | (n = 12 mean) | Difference |
| 5 | 62.908 | 62.844 | 0.0 |
| 10 | 78.274 | 77.681 | 0.4 |
| 15 | 84.979 | 83.751 | 1.5 |
| 20 | 88.935 | 87.857 | 1.2 |
| 30 | 93.181 | 92.242 | 0.9 |
| n (time points)* | | Sum = | 3.9 |
| 5 | | F2 = | 94 |

If F2 ≥ 50, similarly demonstrated profiles

*Number of samplings used where % RSD <20% and to include one time point past 85% mean release

TABLE 71
Tavapadon (CVL-751) 10 mg Tablets
Lot #1: (Coated)
Lot #2: (Uncoated)

| | Lot #1 | Lot #2 | |
|---|---|---|---|
| | % release | % release | Square of the |
| Time point (min) | (n = 12 mean) | (n = 12 mean) | Difference |
| 5 | 61.800 | 62.934 | 1.3 |
| 10 | 77.125 | 78.173 | 1.1 |
| 15 | 84.573 | 84.181 | 0.2 |
| 20 | 88.416 | 87.754 | 0.4 |
| 30 | 93.169 | 92.199 | 0.9 |
| n (time points)* | | Sum = | 3.9 |
| 5 | | F2 = | 94 |

If F2 ≥ 50, similarly demonstrated profiles

*Number of samplings used where % RSD <20% and to include one time point past 85% mean release

TABLE 72
Tavapadon (CVL-751) 15 mg Tablets
Lot #1: (Coated)
Lot #2: (Uncoated)

| | Lot #1 | Lot #2 | |
|---|---|---|---|
| | % release | % release | Square of the |
| Time point (min) | (n = 12 mean) | (n = 12 mean) | Difference |
| 10 | 62.079 | 64.013 | 3.7 |
| 15 | 73.742 | 74.141 | 0.2 |
| 20 | 79.766 | 79.633 | 0.0 |
| 30 | 86.441 | 86.086 | 0.1 |
| 45 | 91.553 | 91.050 | 0.3 |
| n (time points)* | | Sum = | 4.3 |
| 5 | | F2 = | 93 |

If F2 ≥ 50, similarly demonstrated profiles

*Number of samplings used where % RSD <20% and to include one time point past 85% mean release

TABLE 73
Tavapadon (CVL-751) 0.25 mg Tablets
Lot #1: (Medium Particle Size)
Lot #2: (Small Particle Size)

| | Lot #1 | Lot #2 | |
|---|---|---|---|
| | % release | % release | Square of the |
| Time point (min) | (n = 12 mean) | (n = 12 mean) | Difference |
| 5 | 50.776 | 80.611 | 890.1 |
| 10 | 68.209 | 91.441 | 539.7 |
| 15 | 74.847 | 93.220 | 337.6 |
| 20 | 79.804 | 95.009 | 231.2 |
| 30 | 85.278 | 96.224 | 119.8 |
| 45 | 89.415 | 96.542 | 50.8 |
| n (time points)* | | Sum = | 2169.2 |
| 5 | | F2 = | 36 |

If F2 ≥ 50, similarly demonstrated profiles

*Number of samplings used where % RSD <20% and to include one time point past 85% mean release

TABLE 74
Tavapadon (CVL-751) 0.25 mg Tablets
Lot #1: (Medium Particle Size)
Lot #2: (Large Particle Size)

| | Lot #1 | Lot #2 | |
|---|---|---|---|
| | % release | % release | Square of the |
| Time point (min) | (n = 12 mean) | (n = 12 mean) | Difference |
| 5 | 50.776 | 30.186 | 423.9 |
| 10 | 68.209 | 41.316 | 723.2 |
| 15 | 74.847 | 46.868 | 782.8 |
| 20 | 79.804 | 51.478 | 802.4 |
| 30 | 85.278 | 58.187 | 733.9 |
| 45 | 89.415 | 64.397 | 625.9 |
| 60 | 91.791 | 68.466 | 544.1 |
| 75 | 94.008 | 73.813 | 407.8 |
| n (time points)* | | Sum = | 5044.1 |
| 8 | | F2= | 30 |

If F2 ≥ 50, similarly demonstrated profiles

*Number of samplings used where % RSD <20% and to include one time point past 85% mean release

TABLE 75
Tavapadon (CVL-751) 0.25 mg Tablets
Lot #1: (Medium Particle Size)
Lot #2: (Un-milled API)

| | Lot #1 | Lot #2 | |
|---|---|---|---|
| | % release | % release | Square of the |
| Time point (min) | (n = 12 mean) | (n = 12 mean) | Difference |
| 5 | 50.776 | 7.885 | 1839.6 |
| 10 | 68.209 | 11.994 | 3160.1 |
| 15 | 74.847 | 14.737 | 3613.2 |
| 20 | 79.804 | 17.290 | 3908.0 |
| 30 | 85.278 | 21.698 | 4042.4 |
| n (time points)* | | Sum = | 16563.4 |
| 5 | | F2 = | 12 |

If F2 ≥ 50, similarly demonstrated profiles

*Number of samplings used where % RSD <20% and to include one time point past 85% mean release

TABLE 76
Tavapadon (CVL-751) 1 mg Tablets
Lot #1: (Medium Particle Size)
Lot #2: (Small Particle Size)

| | Lot #1 | Lot #2 | |
|---|---|---|---|
| | % release | % release | Square of the |
| Time point (min) | (n = 12 mean) | (n = 12 mean) | Difference |
| 5 | 65.785 | 79.814 | 196.8 |
| 10 | 79.269 | 91.520 | 150.1 |
| 15 | 84.692 | 95.011 | 106.5 |
| 20 | 87.306 | 96.647 | 87.3 |
| n (time points)* | | Sum = | 540.6 |
| 4 | | F2 = | 47 |

If F2 ≥ 50, similarly demonstrated profiles

*Number of samplings used where % RSD <20% and to include one time point past 85% mean release

TABLE 77
Tavapadon (CVL-751) 1 mg Tablets
Lot #1: (Medium Particle Size)
Lot #2: (Large Particle Size)

| | Lot #1 | Lot #2 | |
|---|---|---|---|
| | % release | % release | Square of the |
| Time point (min) | (n = 12 mean) | (n = 12 mean) | Difference |
| 5 | 65.785 | 55.999 | 95.8 |
| 10 | 79.269 | 68.889 | 107.7 |
| 15 | 84.692 | 74.896 | 96.0 |
| 20 | 87.306 | 78.347 | 80.3 |
| 30 | 90.757 | 82.398 | 69.9 |
| 45 | 92.734 | 86.061 | 44.5 |
| 60 | 94.391 | 88.248 | 37.7 |
| n (time points)* | | Sum = | 531.9 |
| 7 | | F2 = | 53 |

If F2 ≥ 50, similarly demonstrated profiles

*Number of samplings used where % RSD <20% and to include one time point past 85% mean release

TABLE 78
Tavapadon (CVL-751) 1 mg Tablets
Lot #1: (Medium Particle Size)
Lot #2: (Un-milled API)

| Time | Lot#1 | Lot#2 | Square |
|---|---|---|---|
| point | % release | % release | of the |
| (min) | (n = 12 mean) | (n = 12 mean) | Difference |
| 5 | 65.785 | 44.932 | 434.8 |
| 10 | 79.269 | 57.339 | 480.9 |
| 15 | 84.692 | 63.522 | 448.2 |
| 20 | 87.306 | 66.715 | 424.0 |
| 30 | 90.757 | 71.463 | 372.3 |
| 45 | 92.734 | 75.575 | 294.4 |
| 60 | 94.391 | 78.400 | 255.7 |
| 75 | 95.365 | 82.656 | 161.5 |
| n (time | | Sum= | 2871.9 |
| points)* | | F2= | 36 |

| | |
|---|---|
| 8 | If F2 ≥ 50, similarly |
| | demonstrated profiles |

*Number of samplings used where % RSD < 20% and to include one time point past 85% mean release

TABLE 79
Tavapadon (CVL-751) 5 mg Tablets
Lot #1: (Medium Particle Size)
Lot #2: (Small Particle Size)

| Time | Lot#1 | Lot#2 | Square |
|---|---|---|---|
| point | % release | % release | of the |
| (min) | (n = 12 mean) | (n = 12 mean) | Difference |
| 5 | 64.648 | 78.174 | 183.0 |
| 10 | 81.439 | 89.476 | 64.6 |
| 15 | 87.525 | 93.523 | 36.0 |
| 20 | 90.167 | 95.183 | 25.2 |
| n (time | | Sum= | 308.7 |
| points)* | | F2= | 53 |

| | |
|---|---|
| 4 | If F2 ≥ 50, similarly |
| | demonstrated profiles |

*Number of samplings used where % RSD < 20% and to include one time point past 85% mean release

TABLE 80
Tavapadon (CVL-751) 5 mg Tablets
Lot #1: (Medium Particle Size)
Lot #2: (Large Particle Size)

| Time | Lot#1 | Lot#2 | Square |
|---|---|---|---|
| point | % release | % release | of the |
| (min) | (n = 12 mean) | (n = 12 mean) | Difference |
| 5 | 64.648 | 56.123 | 72.7 |
| 10 | 81.439 | 72.828 | 74.1 |
| 15 | 87.525 | 79.608 | 62.7 |
| 20 | 90.167 | 83.312 | 47.0 |
| 30 | 93.387 | 87.828 | 30.9 |
| 45 | 95.652 | 91.471 | 17.5 |
| n (time | | Sum= | 304.9 |
| points)* | | F2= | 57 |

| | |
|---|---|
| 6 | If F2 ≥ 50, similarly |
| | demonstrated profiles |

*Number of samplings used where % RSD < 20% and to include one time point past 85% mean release

TABLE 81
Tavapadon (CVL-751) 5 mg Tablets
Lot #1: (Medium Particle Size)
Lot #2: (Unmilled API)

| Time | Lot#1 | Lot#2 | Square |
|---|---|---|---|
| point | % release | % release | of the |
| (min) | (n = 12 mean) | (n = 12 mean) | Difference |
| 5 | 64.648 | 43.098 | 464.4 |
| 10 | 81.439 | 59.494 | 481.6 |
| 15 | 87.525 | 67.105 | 417.0 |
| 20 | 90.167 | 71.576 | 345.6 |
| 30 | 93.387 | 77.467 | 253.4 |
| 45 | 95.652 | 82.779 | 165.7 |
| 60 | 96.733 | 86.306 | 108.7 |
| 75 | 98.052 | 91.331 | 45.2 |
| n (time | | Sum= | 2281.6 |
| points)* | | F2= | 39 |

| | |
|---|---|
| 8 | If F2 ≥ 50, similarly |
| | demonstrated profiles |

*Number of samplings used where % RSD < 20% and to include one time point past 85% mean release

TABLE 82
Tavapadon (CVL-751) 15 mg Tablets
Lot #1: (Medium Particle Size)
Lot #2: (Small Particle Size)

| Time | Lot#1 | Lot#2 | Square |
|---|---|---|---|
| point | % release | % release | of the |
| (min) | (n = 12 mean) | (n = 12 mean) | Difference |
| 5 | 39.888 | 49.099 | 84.8 |
| 10 | 69.032 | 79.601 | 111.7 |
| 15 | 78.772 | 87.727 | 80.2 |
| 20 | 81.986 | 91.621 | 92.8 |
| 30 | 88.205 | 95.174 | 48.6 |
| 45 | 92.028 | 97.962 | 35.2 |
| n (time | | Sum= | 453.4 |
| points)* | | F2= | 53 |

| | |
|---|---|
| 6 | If F2 ≥ 50, similarly |
| | demonstrated profiles |

*Number of samplings used where % RSD < 20% and to include one time point past 85% mean release

TABLE 83
Tavapadon (CVL-751) 15 mg Tablets
Lot #1: (Medium Particle Size)
Lot #2: (Large Particle Size)

| Time | Lot#1 | Lot#2 | Square |
|---|---|---|---|
| point | % release | % release | of the |
| (min) | (n = 12 mean) | (n = 12 mean) | Difference |
| 5 | 39.888 | 33.203 | 44.7 |
| 10 | 69.032 | 59.043 | 99.8 |
| 15 | 78.772 | 68.850 | 98.4 |
| 20 | 81.986 | 74.616 | 54.3 |
| 30 | 88.205 | 81.186 | 49.3 |
| 45 | 92.028 | 86.669 | 28.7 |
| 60 | 94.115 | 90.008 | 16.9 |
| n (time | | Sum= | 392.1 |
| points)* | | F2= | 56 |

| | |
|---|---|
| 7 | If F2 ≥ 50, similarly |
| | demonstrated profiles |

*Number of samplings used where % RSD < 20% and to include one time point past 85% mean release

TABLE 84
Tavapadon (CVL-751) 15 mg Tablets
Lot #1: (Medium Particle Size)
Lot #2: (Un-milled API)

| Time | Lot#1 | Lot#2 | Square |
|---|---|---|---|
| point | % release | % release | of the |
| (min) | (n = 12 mean) | (n = 12 mean) | Difference |
| 10 | 69.032 | 40.851 | 794.2 |
| 15 | 78.772 | 50.079 | 823.3 |
| 20 | 81.986 | 55.961 | 677.3 |
| 30 | 88.205 | 63.516 | 609.5 |
| 45 | 92.028 | 70.640 | 457.4 |
| 60 | 94.115 | 75.388 | 350.7 |
| 75 | 95.748 | 81.540 | 201.9 |
| n (time | | Sum= | 3914.3 |
| points)* | | F2= | 31 |

| | |
|---|---|
| 7 | If F2 ≥ 50, similarly |
| | demonstrated profiles |

*Number of samplings used where % RSD < 20% and to include one time point past 85% mean release

TABLE 85
Tavapadon (CVL-751) 0.25 mg Tablets
Lot #1: (Registration Release)
Lot #2: 1% SSG

| Time | Lot#1 | Lot#2 | Square |
|---|---|---|---|
| point | % release | % release | of the |
| (min) | (n = 12 mean) | (n = 12 mean) | Difference |
| 5 | 50.391 | 57.321 | 48.0 |
| 10 | 72.973 | 71.558 | 2.0 |
| 15 | 81.700 | 79.172 | 6.4 |
| 20 | 86.849 | 83.200 | 13.3 |
| 30 | 91.880 | 88.558 | 11.0 |
| 45 | 95.546 | 92.153 | 11.5 |
| n (time | | Sum= | 92.3 |
| points)* | | F2= | 70 |

| | |
|---|---|
| 6 | If F2 ≥ 50, similarly |
| | demonstrated profiles |

*Number of samplings used where % RSD < 20% and to include one time point past 85% mean release

TABLE 86
Tavapadon (CVL-751) 0.25 mg Tablets
Lot #1: (Registration Release)
Lot #2: 5% SSG

| Time | Lot#1 | Lot#2 | Square |
|---|---|---|---|
| point | % release | % release | of the |
| (min) | (n = 12 mean) | (n = 12 mean) | Difference |
| 5= | 50.391 | 64.136 | 188.9 |
| 10 | 72.973 | 77.653 | 21.9 |
| 15 | 81.700 | 84.729 | 9.2 |
| 20 | 86.849 | 88.374 | 2.3 |
| 30 | 91.880 | 92.951 | 1.1 |
| n (time | | Sum= | 223.5 |
| points)* | | F2= | 59 |

| | |
|---|---|
| 5 | If F2 ≥ 50, similarly |
| | demonstrated profiles |

*Number of samplings used where % RSD < 20% and to include one time point past 85% mean release

TABLE 87
Tavapadon (CVL-751) 1 mg Tablets
Lot #1: (Registration API)
Lot #2: 1% SSG

| Time | Lot#1 | Lot#2 | Square |
|---|---|---|---|
| point | % release | % release | of the |
| (min) | (n = 12 mean) | (n = 12 mean) | Difference |
| 5 | 67.338 | 61.855 | 30.1 |
| 10 | 82.782 | 77.058 | 32.8 |
| 15 | 88.494 | 84.537 | 15.7 |
| 20 | 91.014 | 88.225 | 7.8 |
| n (time | | Sum= | 86.3 |
| points)* | | F2= | 66 |

| | |
|---|---|
| 4 | If F2 ≥ 50, similarly |
| | demonstrated profiles |

*Number of samplings used where % RSD < 20% and to include one time point past 85% mean release

TABLE 88
Tavapadon (CVL-751) 1 mg Tablets
Lot #1: (Registration API)
Lot #2: 5% SSG

| Time | Lot#1 | Lot#2 | Square |
|---|---|---|---|
| point | % release | % release | of the |
| (min) | (n = 12 mean) | (n = 12 mean) | Difference |
| 5 | 67.338 | 63.377 | 15.7 |
| 10 | 82.782 | 77.478 | 28.1 |
| 15 | 88.494 | 84.841 | 13.3 |
| 20 | 91.014 | 88.033 | 8.9 |
| n (time | | Sum= | 66.1 |
| points)* | | F2= | 69 |

| | |
|---|---|
| 4 | If F2 ≥ 50, similarly |
| | demonstrated profiles |

*Number of samplings used where % RSD < 20% and to include one time point past 85% mean release

TABLE 89
Tavapadon (CVL-751) 15 mg Tablets
Lot #1: (Registration API)
Lot #2: 1% SSG

| Time | Lot#1 | Lot#2 | Square |
|---|---|---|---|
| point | % release | % release | of the |
| (min) | (n = 12 mean) | (n = 12 mean) | Difference |
| 5 | 35.863 | 57.055 | 449.1 |
| 10 | 70.754 | 72.544 | 3.2 |
| 15 | 81.522 | 80.142 | 1.9 |
| 20 | 86.644 | 84.659 | 3.9 |
| 30 | 91.629 | 89.903 | 3.0 |
| n (time | | Sum= | 461.1 |
| points)* | | F2= | 51 |

| | |
|---|---|
| 5 | If F2 ≥ 50, similarly |
| | demonstrated profiles |

*Number of samplings used where % RSD < 20% and to include one time point past 85% mean release

TABLE 90
Tavapadon (CVL-751) 15 mg Tablets
Lot #1: (Registration API)
Lot #2: 5% SSG

| Time | Lot#1 | Lot#2 | Square |
|---|---|---|---|
| point | % release | % release | of the |
| (min) | (n = 12 mean) | (n = 12 mean) | Difference |
| 5 | 35.863 | 58.274 | 502.3 |
| 10 | 70.754 | 74.698 | 15.6 |
| 15 | 81.522 | 82.444 | 0.9 |
| 20 | 86.644 | 86.801 | 0.0 |
| 30 | 91.629 | 91.713 | 0.0 |
| n (time | | Sum= | 518.7 |
| points)* | | F2= | 49 |

| | |
|---|---|
| 5 | If F2 ≥ 50, similarly |
| | demonstrated profiles |

*Number of samplings used where % RSD < 20% and to include one time point past 85% mean release

### Example 5: Alternate 15 Mg Formulation

### Manufacturing Procedure:

The manufacturing process involved blending the drug substance, sodium starch glycolate, and intragranular portion magnesium stearate with microcrystalline cellulose or mannitol or pregelatinized starch or Prosolv© SMCC (silicified microcrystalline cellulose), or lactose monohydrate as listed in the formulation table below. The pre-compaction blend was compacted to form ribbons and milled into granules. The granules were blended with extra granular portion magnesium stearate to form a final blend, and the final blend was then compressed into tablets.

TABLE 91
Formulations Studied Containing 15 mg Tavapadon with Batch 2 as Control

| Batch | 1 | 2 | 3 | 4 | 5 | 6 | 7 | 8 | 9 | 10 |
|---|---|---|---|---|---|---|---|---|---|---|
| Tavapadon % | 5 | 5 | 5 | 5 | 5 | 5 | 5 | 5 | 5 | 15 |
| Microcrystalline Cellulose % | 91 | 59.08 | 59.08 | 59.08 | — | — | — | — | — | 54.08 |
| Lactose Monohydrate % | — | 31.92 | — | — | 31.92 | — | — | — | — | 26.92 |
| Sodium Starch Glycolate % | 3 | 3 | 3 | 3 | 3 | 3 | 3 | 3 | 3 | 3 |
| Magnesium Stearate % | 1 | 1 | 1 | 1 | 1 | 1 | 1 | 1 | 1 | 1 |
| (Non-Bovine) | | | | | | | | | | |
| Mannitol % | — | — | 31.92 | — | — | 91 | — | — | 45.5 | — |
| Pre-gelatinized Starch % | — | — | — | 31.92 | 59.08 | — | 91 | 45.5 | 45.5 | — |
| Prosolv ® SMCC % | — | — | — | — | — | — | — | 45.5 | — | — |

### Tableting Data:

Batches 1-9 were about 300 mg tablets and batch 10 was an about 100 mg tablet. Each batch was compressed into tablets and the compression force evaluated during each batch indicated that the formulation containing starch (batch 5, 7 and 9) required higher forces compared to other formulations to form tablets and had longer disintegration time for tablets.

TABLE 92

| | Weight, | Hardness, | Thickness | Force, |
|---|---|---|---|---|
| Compression | mg | kP | mm | kN |
| Batch 1 | 310 | 12.9 | 4.65 | 5.3 |
| | 300 | 10 | 4.65 | 5.3 |
| | 304 | 11.9 | 4.65 | 5.3 |
| | 302 | 11.3 | 4.65 | 5.2 |
| | 306 | 12.6 | 4.65 | 5 |
| Batch 2 | 297 | 11.7 | 4.11 | 7-9 kN |
| (Control) | 302 | 11.3 | 4.19 | 7-9 kN |
| | 302 | 7.3 | 4.33 | 7-9 kN |
| | 309 | 10.5 | 4.19 | 10.5 |
| | 305 | 11.6 | 4.2 | 10--11 |
| | 306 | 11.7 | 4.2 | 10--11 |
| | 305 | 12.4 | 4.19 | 10--11 |
| | 308 | 13.5 | 4.2 | 10--11 |
| | 306 | 12.7 | 4.18 | 10--11 |
| Batch 3 | 299 | 10.2 | 4.23 | 9.5 |
| | 296 | 9 | 4.21 | 9 |
| | 297 | 8.4 | 4.25 | 8.8 |
| | 299 | 8.5 | 4.26 | 8.9 |
| | 299 | 10.2 | 4.18 | 10.4 |
| Batch 4 | 296 | 8.1 | 4.22 | 11 |
| | 295 | 9.2 | 4.24 | 9.5 |
| | 292 | 7.9 | 4.24 | 10.1 |
| | 296 | 8 | 4.23 | 9.6 |
| | 299 | 8.9 | 4.25 | 10.1 |
| | 288 | 10.4 | 4.11 | 12.8 |
| | 292 | 9.8 | 4.09 | 14.1 |
| | 294 | 9.5 | 4.08 | 14.8 |
| | 290 | 10 | 4.08 | 14.2 |
| | 296 | 11.1 | 4.08 | 13.6 |
| Batch 5 | 296 | 4.9 | 4.2 | 17-18 kN |
| | 303 | 4.1 | 4.2 | 17-18 kN |
| | 302 | 3.8 | 4.23 | 17-18 kN |
| | 301 | 3.4 | 4.22 | 17-18 kN |
| | 300 | 4.2 | 4.21 | 17-18 kN |
| Batch 6 | 291 | 10.1 | 4.34 | 9.8 |
| | 298 | 11.2 | 4.38 | 10.9 |
| | 294 | 10.8 | 4.37 | 10.8 |
| | 296 | 10.7 | 4.38 | 10.7 |
| | 299 | 10.8 | 4.38 | 11.1 |
| Batch 7 | 294 | 2.2 | 4.24 | 19.6 |
| | 296 | 2.1 | 4.21 | 21.2 |
| | 292 | 1.4 | 4.22 | 19.8 |
| | 295 | 2 | 4.22 | 19.8 |
| | 292 | 2.1 | 4.24 | 20.4 |
| Batch 8 | 299 | 8.7 | 4.27 | 10.4 |
| | 300 | 7.3 | 4.27 | 10.8 |
| | 297 | 6.8 | 4.26 | 10.8 |
| | 298 | 9.5 | 4.26 | 10.8 |
| | 298 | 6.8 | 4.26 | 10.6 |
| | 296 | 8.1 | 4.17 | 13.6 |
| | 300 | 9.4 | 4.17 | 14.3 |
| | 299 | 7.5 | 4.19 | 13.8 |
| | 300 | 11 | 4.17 | 13.6 |
| | 298 | 9.1 | 4.17 | 13.4 |
| Batch 9 | 297 | 8.4 | 4.19 | 18-20 kN |
| | 295 | 6.3 | 4.2 | 18-20 kN |
| | 294 | 6.6 | 4.21 | 18-20 kN |
| | 300 | 9 | 4.25 | 18-20 kN |
| | 299 | 8.8 | 4.25 | 18-20 kN |
| | 299 | 5.9 | 4.34 | 13-14 kN |
| | 301 | 6.6 | 4.35 | 13-14 kN |
| | 298 | 6 | 4.36 | 13-14 kN |
| | 297 | 6 | 4.35 | 13-14 kN |
| | 299 | 6.7 | 4.35 | 13-14 kN |
| Batch 10 | 104 | 7.4 | 3.08 | 5.6 |
| | 102 | 6 | 3.09 | na |
| | 103 | 6.9 | 3.12 | 6.4 |
| | 101 | 6 | 3.08 | 6.4 |

### Disintegration Time (Seconds):

Formulation containing starch had the longest disintegration time and the formulations with MCC had shorter disintegration times. All formulations achieved disintegration within 15 minutes and were considered acceptable per USP <701> for an immediate release formulation.

TABLE 93A

| | Batch | | | | | | |
|---|---|---|---|---|---|---|---|
| | 1 | 2 | 2 | 3 | 4 | 4 | 5 |
| | Force (kN) | | | | | | |
| | 5 | 7-9 | 10-11 | 8-9 | 9-11 | 13-14 | 17-18 |
| 1 | 7 | 14 | 17 | 21 | 53 | 74 | 30 |
| 2 | 7 | 14 | 17 | 21 | 51 | 80 | 31 |
| 3 | 7 | 14 | 17 | 21 | 51 | 76 | 31 |
| 4 | 8 | 14 | 17 | 21 | 56 | 81 | 32 |
| 5 | 8 | 14 | 17 | 21 | 55 | 72 | 32 |
| 6 | 8 | 14 | 17 | 21 | 55 | 73 | 34 |
| mean | 7.5 | 14.0 | 17.0 | 21.0 | 53.5 | 76.0 | 31.7 |

TABLE 93B

| | Batch | | | | | | |
|---|---|---|---|---|---|---|---|
| | 6 | 7 | 8 | 8 | 9 | 9 | 10 |
| | Force (kN) | | | | | | |
| | 9-11 | 18-20 | 10-11 | 12-15 | 13-14 | 18-20 | 5-7 |
| 1 | 194 | 230 | 131 | 142 | 200 | 163 | 17 |
| 2 | 196 | 235 | 175 | 148 | 132 | 165 | 17 |
| 3 | 200 | 236 | 126 | 142 | 135 | — | 18 |
| 4 | 198 | 236 | 130 | 148 | 140 | — | 21 |
| 5 | 187 | 232 | 131 | 146 | — | — | 22 |
| 6 | 194 | 225 | 130 | 146 | — | — | 22 |
| mean | 194.8 | 232.3 | 137.2 | 145.3 | 151.8 | 164.0 | 19.5 |

### Dissolution Methods

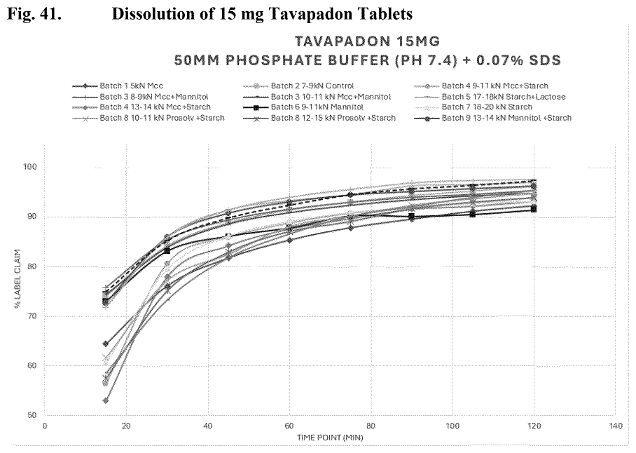

A 120-minute dissolution method was developed for 0.25, 1, 5, 10 and 15 mg tablets, with the dissolution followed by dissolution followed by isocratic reversed phase high performance liquid chromatography (HPLC) with ultraviolet (UV) detection. The method consisted of900 mL of 50 mM sodium phosphate buffer, pH 7.4±0.05 with 0.07 (w/v) SDS at 37±0.5° C. with USP Apparatus 2 (paddles) at 75 rpm. Samples were collected at 15, 30, 45, 60, 75, 90, 105 and 120 minutes, with additional sampling times possible. Detailed method conditions for the 120-minute proposed dissolution method are provided in Table 94 and results are shown in FIG. 41.

TABLE 95
Proposed 120-minute Dissolution Method

| | |
|---|---|
| Apparatus | USP/Ph. Eur. Dissolution Apparatus 2 (Paddle) |
| | and fraction collector |
| Rotation speed | 75 RPM ± 4% |
| Dissolution Medium | 50 mM sodium phosphate buffer, pH 7.4 ± |
| | 0.05 with 0.07% (w/v) SDS |
| Medium Volume | 900 mL |
| Medium Temperature | 37 ± 0.5° C. |
| Filter | Cannula Filters, Polyethylene, 1 μm, Sotax |
| | F100-3301 or equivalent |
| Sampling Volume | Automatic: 1.5 mL |
| | Manual: 5 mL |
| Sampling Times | 15, 30, 45, 60, 75, 90, 105 and 120 minutes |
| | (additional sampling times are possible) |

All batches achieved at least 85% drug release by 60 minutes when tested under USP II dissolution apparatus, and 50 mM Phosphate Buffer media w/suitable surfactant.

TABLE 95

| | Time (minutes) | | | | | | | |
|---|---|---|---|---|---|---|---|---|
| | 15 | 30 | 45 | 60 | 75 | 90 | 105 | 120 |
| Batch 1 | | | | | | | | |
| 5 kN | | | | | | | | |
| Mean | 64.4 | 76.1 | 81.7 | 85.3 | 87.8 | 89.5 | 91.1 | 92.2 |
| % RSD | 0.20% | 0.50% | 0.50% | 0.60% | 1.00% | 0.70% | 0.90% | 0.90% |
| Batch 2 | | | | | | | | |
| 7-9 kN | | | | | | | | |
| Mean | 74.1 | 84.2 | 88.8 | 91.4 | 93 | 94.3 | 95.2 | 96.2 |
| % RSD | 3.10% | 2.00% | 1.40% | 1.40% | 1.20% | 0.90% | 0.70% | 1.00% |
| Batch 2 | | | | | | | | |
| 10-11 kN | | | | | | | | |
| Mean | 74.9 | 85.2 | 89.8 | 92.4 | 94.5 | 95.7 | 96.4 | 97.3 |
| % RSD | 0.70% | 0.70% | 0.80% | 0.70% | 0.60% | 0.70% | 0.50% | 0.60% |
| Batch 3 | | | | | | | | |
| 8-9 kN | | | | | | | | |
| Mean | 75.8 | 85.4 | 89.4 | 91.6 | 93 | 94 | 94.6 | 95.4 |
| % RSD | 1.90% | 2.10% | 2.10% | 1.90% | 1.90% | 2.30% | 2.30% | 2.20% |
| Batch 3 | | | | | | | | |
| 10-11 kN | | | | | | | | |
| Mean | 74.4 | 83.9 | 88.6 | 90.9 | 92.4 | 93.5 | 94.3 | 94.8 |
| % RSD | 0.20% | 0.50% | 0.60% | 0.80% | 0.80% | 0.90% | 0.90% | 1.00% |
| Batch 4 | | | | | | | | |
| 9-11 kN | | | | | | | | |
| Mean | 56.4 | 80.6 | 85.9 | 88.8 | 90.8 | 91.9 | 92.9 | 93.9 |
| % RSD | 0.60% | 1.70% | 1.50% | 1.60% | 2.20% | 1.90% | 1.90% | 1.70% |
| Batch 4 | | | | | | | | |
| 13-14 kN | | | | | | | | |
| Mean | 52.9 | 77.9 | 84.2 | 87.6 | 89.7 | 91.6 | 92.2 | 93.2 |
| % RSD | 0.80% | 0.20% | 1.00% | 0.70% | 0.80% | 1.00% | 1.00% | 1.30% |
| Batch 5 | | | | | | | | |
| 17-18 kN | | | | | | | | |
| Mean | 72 | 86 | 91.4 | 93.2 | 94.4 | 96.2 | 96.5 | 96.9 |
| % RSD | 2.20% | 0.20% | 0.20% | 0.80% | 1.30% | 0.30% | 0.60% | 0.30% |
| Batch 6 | | | | | | | | |
| 9-11 kN | | | | | | | | |
| Mean | 73 | 83.1 | 86.1 | 87.8 | 90.2 | 90.1 | 90.5 | 91.4 |
| % RSD | 0.70% | 0.80% | 2.40% | 2.30% | 1.30% | 2.20% | 1.20% | 1.30% |
| Batch 7 | | | | | | | | |
| 18-20 kN | | | | | | | | |
| Mean | 60.6 | 79.6 | 86.2 | 89.2 | 91 | 92 | 92.8 | 93.2 |
| % RSD | 1.20% | 1.00% | 1.30% | 1.20% | 1.40% | 1.40% | 1.40% | 1.40% |
| Batch 8 | | | | | | | | |
| 10-11 kN | | | | | | | | |
| Mean | 61.6 | 77.2 | 82.5 | 88.1 | 90.2 | 92.4 | 93.7 | 94.8 |
| % RSD | 1.30% | 2.00% | 5.40% | 2.20% | 1.70% | 1.60% | 1.40% | 1.00% |
| Batch 8 | | | | | | | | |
| 12-15 kN | | | | | | | | |
| Mean | 57.5 | 75.1 | 82.9 | 87.1 | 89.1 | 91.7 | 93 | 93.9 |
| % RSD | 2.30% | 0.40% | 0.30% | 0.10% | 0.20% | 0.30% | 0.20% | 0.20% |
| Batch 9 | | | | | | | | |
| 13-14 kN | | | | | | | | |
| Mean | 72.7 | 85.9 | 90.7 | 93 | 94.4 | 95.1 | 95.7 | 96.2 |
| % RSD | 2.10% | 1.10% | 0.90% | 0.80% | 0.80% | 1.00% | 0.60% | 1.00% |
| Batch 9 | | | | | | | | |
| 18-20 kN | | | | | | | | |
| Mean | 71.8 | 85.8 | 91.3 | 93.9 | 95.5 | 96.8 | 97.3 | 97.5 |
| % RSD | 7.30% | 5.90% | 5.00% | 4.20% | 3.60% | 3.30% | 3.30% | 3.20% |
| Batch 10 | | | | | | | | |
| 5-7 kN | | | | | | | | |
| Mean | 58.5 | 73.3 | 81.8 | 86.6 | 90.2 | 92.1 | 94 | 95.4 |
| % RSD | 1.00% | 0.80% | 1.20% | 1.30% | 1.40% | 1.50% | 1.70% | 1.80% |

### Example 6: Alternate 0.25 Mg Formulation

### Manufacturing Procedure:

The manufacturing process involved blending the drug substance, sodium starch glycolate, and intragranular portion magnesium stearate with microcrystalline cellulose or mannitol or pregelatinized starch or Prosolv® SMCC (silicified microcrystalline cellulose), or lactose monohydrate as listed in the formulation table below. The pre-compaction blend was compacted to form ribbons and milled into granules. The granules were blended with extra granular portion magnesium stearate to form a final blend, and the final blend was then compressed into tablets.

TABLE 96
0.25% Tavapadon Formulations with Batch 2 as Control

| Batch | 1 | 2 | 3 | 4 | 5 | 6 | 7 | 8 | 9 | 10 | 11 |
|---|---|---|---|---|---|---|---|---|---|---|---|
| Tavapadon % | 0.25 | 0.25 | 0.25 | 0.25 | 0.25 | 0.25 | 0.25 | 0.25 | 0.25 | 0.25 | 0.25 |
| Microcrystalline | 63.83 | 76.596 | 51.064 | 63.23 | 64.43 | 63.63 | 64.03 | 63.83 | 63.83 | 65.33 | 66.83 |
| Cellulose % | | | | | | | | | | | |
| Lactose | 31.92 | 19.404 | 44.936 | 31.92 | 31.92 | 31.92 | 31.92 | 31.92 | 31.92 | 31.92 | 31.92 |
| Monohydrate % | | | | | | | | | | | |
| Sodium Starch | 3 | 3 | 3 | 3.6 | 2.4 | 3 | 3 | 3 | 3 | 1.5 | 0 |
| Glycolate % | | | | | | | | | | | |
| Magnesium | 1 | 1 | 1 | 1 | 1 | 1.2 | 0.8 | 1 | 1 | 1 | 1 |
| Stearate % | | | | | | | | | | | |
| (Non-Bovine) | | | | | | | | | | | |

We claim:

1. A pharmaceutical tablet comprising:
(a) about 0.25% by weight to about 15% by weight of tavapadon free base; and
(b) at least about 80% by weight of at least one filler.

2. The pharmaceutical tablet of claim 1 comprising:
(a) about 0.25% by weight to about 5% by weight of tavapadon free base; and
(b) at least about 90% by weight of at least one filler.

3. The pharmaceutical tablet of claim 2, wherein at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof.

4. The pharmaceutical tablet of claim 3, wherein at least one filler is cellulose.

5. The pharmaceutical tablet of claim 3, wherein at least one filler is dibasic calcium phosphate.

6. The pharmaceutical tablet of claim 4, wherein the cellulose is microcrystalline cellulose.

7. The pharmaceutical tablet of claim 3, wherein at least one filler is lactose.

8. The pharmaceutical tablet of claim 7, wherein the lactose is lactose monohydrate.

9. The pharmaceutical tablet of claim 3, wherein the filler comprises cellulose and lactose.

10. The pharmaceutical tablet of claim 9, wherein the cellulose is microcrystalline cellulose and the lactose is lactose monohydrate.

11. The pharmaceutical tablet of claim 10, wherein the filler comprises about 59% to about 64% by weight of microcrystalline cellulose and about 31% to about 33% by weight of lactose monohydrate.

12. The pharmaceutical tablet of claim 11, wherein a coating is added to the pharmaceutical tablet.

13. The pharmaceutical tablet of claim 12, wherein the coating comprises a polymer coating.

14. The pharmaceutical tablet of claim 13, wherein the coating comprises polyvinyl alcohol, polyethylene glycol, titanium dioxide, and talc.

15. The pharmaceutical tablet of claim 3, further comprising at least one disintegrant.

16. The pharmaceutical tablet of claim 15, wherein at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof.

17. The pharmaceutical tablet of claim 16, wherein at least one disintegrant is present in an amount of about 3% by weight.

18. The pharmaceutical tablet of claim 17, wherein at least one disintegrant is sodium starch glycolate.

19. The pharmaceutical tablet of claim 18, wherein a coating is added to the pharmaceutical tablet.

20. The pharmaceutical tablet of claim 19, wherein the coating comprises a polymer coating.

21. The pharmaceutical tablet of claim 20, wherein the coating comprises polyvinyl alcohol, polyethylene glycol, titanium dioxide, and talc.

22. The pharmaceutical tablet of claim 3, further comprising at least one lubricant.

23. The sold oral dosage form of claim 22, wherein at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof.

24. The pharmaceutical tablet of claim 23, wherein at least one lubricant is present in an amount of about 1% by weight.

25. The pharmaceutical tablet of claim 24, wherein at least one lubricant is magnesium stearate.

26. The pharmaceutical tablet of claim 25, wherein a coating is added to the pharmaceutical tablet.

27. The pharmaceutical tablet of claim 26, wherein the coating comprises a polymer coating.

28. The pharmaceutical tablet of claim 27, wherein the coating comprises polyvinyl alcohol, polyethylene glycol, titanium dioxide, and talc.

29. The pharmaceutical tablet of claim 3, further comprising:
(c) at least one disintegrant; and
(d) at least one lubricant.

30. The pharmaceutical tablet of claim 29, wherein:
(b) at least one filler is selected from the group consisting of lactose, anhydrous lactose, lactose monohydrate, lactitol, maltitol, mannitol, sorbitol, xylitol, dextrose, dextrose monohydrate, fructose, sucrose, sucrose-based diluents, compressible sugar, confectioner's sugar, sugar spheres, maltose, inositol, hydrolyzed cereal solids, starches, corn starch, wheat starch, rice starch, potato starch, tapioca starch, starch components, amylose, dextrates, modified or processed starches, pregelatinized starch, dextrins, cellulose, powdered cellulose, microcrystalline cellulose, silicified microcrystalline cellulose, food grade sources of α- and amorphous cellulose and powdered cellulose, cellulose acetate, calcium salts, calcium carbonate, tribasic calcium phosphate, dicalcium phosphate, dibasic calcium phosphate, monobasic calcium sulfate monohydrate, calcium sulfate, granular calcium lactate trihydrate, magnesium carbonate, magnesium oxide, bentonite, kaolin, sodium chloride, and mixtures thereof;
(c) at least one disintegrant is selected from the group consisting of starches, pregelatinized starch, sodium starch glycolate, clays, magnesium aluminum silicate, cellulose-based disintegrants, powdered cellulose, microcrystalline cellulose, methylcellulose, low-substituted hydroxypropylcellulose, carmellose, carmellose calcium, carmellose sodium, croscarmellose sodium, alginates, povidone, crospovidone, polacrilin potassium, gums, agar, guar gum, locust bean gum, gum karaya, pectin, tragacanth gum, colloidal silicon dioxide, and mixtures thereof; and
(d) at least one lubricant is selected from the group consisting of glyceryl behenate, stearic acid and salts thereof, magnesium stearate, calcium stearate, sodium stearate, hydrogenated vegetable oils, glyceryl palmitostearate, talc, waxes, sodium benzoate, sodium acetate, sodium fumarate, sodium stearyl fumarate, PEGs, PEG 4000, PEG 6000, poloxamers, polyvinyl alcohol, sodium oleate, sodium lauryl sulfate, magnesium lauryl sulfate, and mixtures thereof.

31. The pharmaceutical tablet of claim 30, wherein at least one disintegrant is present in an amount of about 3% by weight and at least one lubricant is present in an amount of about 1% by weight.

32. The pharmaceutical tablet of claim 31, wherein:
(b) at least one filler comprises lactose and cellulose;
(c) at least one disintegrant is a starch; and
(d) at least one lubricant is stearic acid or salts thereof.

33. The pharmaceutical tablet of claim 31, wherein:
(b) at least one filler comprises lactose monohydrate and microcrystalline cellulose;
(c) at least one disintegrant is sodium starch glycolate; and
(d) at least one lubricant is magnesium stearate.

34. The pharmaceutical tablet of claim 33, wherein a coating is added to the pharmaceutical tablet.

35. The pharmaceutical tablet of claim 34, wherein the coating comprises a polymer coating.

36. The pharmaceutical tablet of claim 35, wherein the coating comprises polyvinyl alcohol, polyethylene glycol, titanium dioxide, and talc.

37. The pharmaceutical tablet of claim 33, comprising:
(a) about 0.25% by weight of tavapadon;
(b) about 64% by weight of microcrystalline cellulose;
(c) about 32% by weight of lactose monohydrate;
(d) about 3% sodium starch glycolate; and
(e) about 1% magnesium stearate.

38. The pharmaceutical tablet of claim 37, wherein the pharmaceutical tablet comprises about 0.25 mg of tavapadon.

39. The pharmaceutical tablet of claim 38, wherein the pharmaceutical tablet comprises:
(b) about 63.83 mg of microcrystalline cellulose;
(c) about 31.92 mg of lactose monohydrate;
(d) about 3 mg sodium starch glycolate; and
(e) about 1 mg magnesium stearate.

40. The pharmaceutical tablet of claim 39, wherein a coating is added to the pharmaceutical tablet.

41. The pharmaceutical tablet of claim 40, wherein the coating comprises a polymer coating.

42. The pharmaceutical tablet of claim 41, wherein the coating comprises polyvinyl alcohol, polyethylene glycol, titanium dioxide, and talc.

43. The pharmaceutical tablet of claim 33, comprising:
(a) about 1% by weight of tavapadon;
(b) about 63% by weight of microcrystalline cellulose;
(c) about 32% by weight of lactose monohydrate;
(d) about 3% sodium starch glycolate; and
(e) about 1% magnesium stearate.

44. The pharmaceutical tablet of claim 43, wherein the pharmaceutical tablet comprises about 1 mg of tavapadon.

45. The pharmaceutical tablet of claim 44, wherein the pharmaceutical tablet comprises:
(b) about 63.08 mg of microcrystalline cellulose;
(c) about 31.92 mg of lactose monohydrate;
(d) 3 about mg sodium starch glycolate; and
(e) about 1 mg magnesium stearate.

46. The pharmaceutical tablet of claim 45, wherein a coating is added to the pharmaceutical tablet.

47. The pharmaceutical tablet of claim 46, wherein the coating comprises a polymer coating.

48. The pharmaceutical tablet of claim 47, wherein the coating comprises polyvinyl alcohol, polyethylene glycol, titanium dioxide, and talc.

49. The pharmaceutical tablet of claim 33, comprising:
(a) about 5% by weight of tavapadon;
(b) about 59% by weight of microcrystalline cellulose;
(c) about 32% by weight of lactose monohydrate;
(d) about 3% sodium starch glycolate; and
(e) about 1% magnesium stearate.

50. The pharmaceutical tablet of claim 49, wherein the pharmaceutical tablet comprises about 5 mg of tavapadon.

51. The pharmaceutical tablet of claim 49, wherein the pharmaceutical tablet comprises about 15 mg of tavapadon.

52. The pharmaceutical tablet of claim 50, wherein the pharmaceutical tablet comprises:
(b) about 59.08 mg of microcrystalline cellulose;
(c) about 31.92 mg of lactose monohydrate;
(d) about 3 mg sodium starch glycolate; and
(e) about 1 mg magnesium stearate.

53. The pharmaceutical tablet of claim 52, wherein a coating is added to the pharmaceutical tablet.

54. The pharmaceutical tablet of claim 53, wherein the coating comprises a polymer coating.

55. The pharmaceutical tablet of claim 54, wherein the coating comprises polyvinyl alcohol, polyethylene glycol, titanium dioxide, and talc.

56. The pharmaceutical tablet of claim 51, wherein the pharmaceutical tablet comprises:
(b) about 177.24 mg of microcrystalline cellulose;
(c) about 95.76 mg of lactose monohydrate;
(d) about 9 mg sodium starch glycolate; and
(e) about 3 mg magnesium stearate.

57. The pharmaceutical tablet of claim 56, wherein a coating is added to the pharmaceutical tablet.

58. The pharmaceutical tablet of claim 57, wherein the coating comprises a polymer coating.

59. The pharmaceutical tablet of claim 58, wherein the coating comprises polyvinyl alcohol, polyethylene glycol, titanium dioxide, and talc.

60. The pharmaceutical tablet of claim 49, wherein the pharmaceutical tablet comprises about 10 mg of tavapadon.

61. The pharmaceutical tablet of claim 60, wherein the pharmaceutical tablet comprises:
(b) about 118.16 mg of microcrystalline cellulose;
(c) about 63.84 mg of lactose monohydrate;
(d) about 6 mg sodium starch glycolate; and
(e) about 2 mg magnesium stearate.

62. The pharmaceutical tablet of claim 61, wherein a coating is added to the pharmaceutical tablet.

63. The pharmaceutical tablet of claim 62, wherein the coating comprises a polymer coating.

64. The pharmaceutical tablet of claim 63, wherein the coating comprises polyvinyl alcohol, polyethylene glycol, titanium dioxide, and talc.

65. The pharmaceutical tablet of any one of claims 1-36, wherein the pharmaceutical tablet comprises about 0.25 mg of tavapadon.

66. The pharmaceutical tablet of any one of claims 1-36, wherein the pharmaceutical tablet comprises about 1 mg of tavapadon.

67. The pharmaceutical tablet of any one of claims 1-36, wherein the pharmaceutical tablet comprises about 5 mg of tavapadon.

68. The pharmaceutical tablet of any one of claims 1-36, wherein the pharmaceutical tablet comprises about 10 mg of tavapadon.

69. The pharmaceutical tablet of any one of claims 1-36, wherein the pharmaceutical tablet comprises about 15 mg of tavapadon.

70. A pharmaceutical tablet comprising tavapadon and at least one filler; wherein the tavapadon is comprised of particles having an average D[4,3] of about 12 μm.

71. The pharmaceutical tablet of claim 70, wherein the pharmaceutical tablet comprises about 0.25 mg of tavapadon.

72. The pharmaceutical tablet of claim 70, wherein the pharmaceutical tablet comprises about 1 mg of tavapadon.

73. The pharmaceutical tablet of claim 70, wherein the pharmaceutical tablet comprises about 5 mg of tavapadon.

74. The pharmaceutical tablet of claim 70, wherein the pharmaceutical tablet comprises about 10 mg of tavapadon.

75. The pharmaceutical tablet of claim 70, wherein the pharmaceutical tablet comprises about 15 mg of tavapadon.

76. A pharmaceutical tablet comprising tavapadon and at least one filler, wherein about 75% or more of the tavapadon is released in 20 minutes in 0.01N HCl with 0.15% or 0.2% SDS.

77. The pharmaceutical tablet of claim 76, wherein the pharmaceutical tablet comprises about 0.25 mg of tavapadon.

78. The pharmaceutical tablet of claim 76, wherein the pharmaceutical tablet comprises about 1 mg of tavapadon.

79. The pharmaceutical tablet of claim 76, wherein the pharmaceutical tablet comprises about 5 mg of tavapadon.

80. The pharmaceutical tablet of claim 76, wherein the pharmaceutical tablet comprises about 10 mg of tavapadon.

81. The pharmaceutical tablet of claim 76, wherein the pharmaceutical tablet comprises about 15 mg of tavapadon.

82. A method of manufacturing a pharmaceutical tablet comprising tavapadon and at least one filler; the steps comprising:
(a) blending tavapadon with filler to obtain a pre-compaction blend;
(b) compacting the pre-compaction blend to form ribbons;
(c) milling the ribbons into granules; and
(d) compressing into tablets.

83. The method of claim 82, further comprising (a) blending tavapadon with filler and disintegrant to obtain the pre-compaction blend.

84. The method of claim 82, further comprising (a) blending tavapadon with filler, disintegrant, and intragranular portion lubricant to obtain the pre-compaction blend.

85. A method of manufacturing a pharmaceutical tablet, the steps comprising
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets.

86. The method of manufacturing of claim 85, wherein the disintegrant is sodium starch glycolate.

87. The method of manufacturing of claim 86, wherein the lubricant is magnesium stearate.

88. The method of manufacturing of claim 87, wherein the filler is microcrystalline cellulose and lactose monohydrate.

89. A pharmaceutical tablet comprising tavapadon and at least one filler wherein disintegration of the tablet is achieved within 15 minutes under guidelines and requirements of United States Pharmacopeia (USP) general chapter <701>.

90. A pharmaceutical tablet comprising tavapadon and at least one filler, manufactured by:
(a) blending tavapadon with filler to obtain a pre-compaction blend;
(b) compacting the pre-compaction blend to form ribbons;
(c) milling the ribbons into granules; and
(d) compressing into tablets.

91. The pharmaceutical tablet of claim 90, manufactured by:
(a) blending tavapadon with filler and disintegrant to obtain the pre-compaction blend.

92. The pharmaceutical tablet of claim 90, manufactured by:
(a) blending tavapadon with filler, disintegrant, and intragranular portion lubricant to obtain the pre-compaction blend.

93. A pharmaceutical tablet comprising tavapadon and at least one filler; manufactured by
(a) blending tavapadon, disintegrant, and intragranular portion lubricant with filler to obtain a pre-compaction blend;
(b) compacting the pre-compaction blend to form ribbons;
(c) milling the ribbons into granules;
(d) blending the granules with extra granular portion lubricant to form a final blend; and
(e) compressing the final blend into tablets.

94. The pharmaceutical tablet of claim 93, wherein the disintegrant is sodium starch glycolate.

95. The pharmaceutical tablet of claim 93, wherein the lubricant is magnesium stearate.

96. The pharmaceutical tablet of claim 93, wherein the filler is microcrystalline cellulose and lactose monohydrate.

97. A pharmaceutical tablet comprising tavapadon and at least one filler, wherein 90% or more dissolution of the tablet is achieved under guidelines and requirements of United States Pharmacopeia (USP) general chapter <711>.

98. A method for treating Parkinson's Disease (PD) in a patient, comprising administering to the patient a pharmaceutical formulation comprising:
(a) about 0.25% by weight to about 15% by weight of tavapadon free base; and
(b) at least about 80% by weight of at least one filler; wherein the Parkinson's Disease is effectively treated.

99. A pharmaceutical tablet comprising tavapadon and at least one filler; wherein the tavapadon is comprised of particles having an average D[4,3] of about <25 μm.

100. The pharmaceutical tablet of claim 99, wherein the pharmaceutical tablet comprises about 0.25 mg of tavapadon.

101. The pharmaceutical tablet of claim 99, wherein the pharmaceutical tablet comprises about 1 mg of tavapadon.

102. The pharmaceutical tablet of claim 99, wherein the pharmaceutical tablet comprises about 5 mg of tavapadon.

103. The pharmaceutical tablet of claim 99, wherein the pharmaceutical tablet comprises about 10 mg of tavapadon.

104. The pharmaceutical tablet of claim 99, wherein the pharmaceutical tablet comprises about 15 mg of tavapadon.
